SEQUENCE LISTING (195 sequences)

SEQ ID NO: 1 - protein, 163 aa, Chlamydomonas reinhardtii
MNTKYNKEFLLYLAGFVDGDGSIIAQIKPNQSYKFKHQLSLTFQVTQKTQRRWFLDKLVDEIGVGYVRDRGSVSDYILSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 2 - DNA, 24 bp, Artificial Sequence
  Description of Artificial Sequence Synthetic I-CreI C1221 target sequence
tcaaaacgtcgtacgacgttttga

SEQ ID NO: 3 - DNA, 4145 bp, Homo sapiens
gaagagcaagcgccatgttgaagccatcattaccattcacatccctcttattcctgcagctgcccctgctgggagtggggctgaacacgacaattctgacgcccaatgggaatgaagacaccacagctggtgggaaatctgggactggagggggctggtgagaagggtggctgtgggaaggggccgtacagagatctggtgcctgccactggccattacaatcatgtgggcagaattgaaaagtggagtgggaagggcaagggggagggttccctgcctcacgctacttcttctttctttcttgtttgtttgtttctttctttcttttgaggcagggtctcactatgttgcctaggctggtctcaaactcctggcctctagtgatcctcctgcctcagcctttcaaagcaccaggattacagacatgagccaccgtgcttggcctcctccttctgaccatcatttctctttccctccctgccttcattttctccccaatctagatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtatgagaagggacgagggggaggggatgaagaagaggtgggttggatcagagaccaagagagagggtagcaagtctcccaggtaccccactgttttctcctggggtaagtcataagtcggttgaggggagatgaggctaggctctggatatctgcagtacccagattggccccactgttcctcttccttccaacctttctcctctaggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctgggtaatttggaaagaaagggtcaagagaccagggatactgtgggacattggagtctacagagtagtgttcttttatcataagggtacatgggcagaaaagaggaggtaggggatcatgatgggaagggaggaggtattaggggcactaccttcaggatcctgacttgtctaggccaggggaatgaccacatatgcacacatatctccagtgatcccctgggctccagagaacctaacacttcacaaactgagtgaatcccagctagaactgaactggaacaacagattcttgaaccactgtttggagcacttggtgcagtaccggactgactgggaccacagctggactgtgagtgactagggacgtgaatgtagcagctaaggccaagaaagtagggctaaaggattcaaccagacagatagaaggacctaatatcaagctcctgttctctgcctcccagcttctctgctcaccccctaccctccctcctccaactcctttcccccctattttctccagtgagttttctttttttcttttcttttctttctttctttcttttttttttttttttgagacagagcctcactctgttgcccaggcttgagtgcagtggggcgatcttgggctcactgcgacctctgtctccctggttcaagtgattctcctgcttcagcctcccaagtagctgggagcatgcacaaccatgcctggctaatttttgtatttttagtaaagacagggttttgccatgttggtcaggctggtcttgaactcctgacctcaggtgatctgcccacctcggcctcccaaagtgctgggattacaggcgtgagccaccattcctgacaccagtgagttttcattagggattccctacccatactcttcctgataccagatagacaagtaaacaaaaggaagccattaaggggatccagaggggaggcattagattcaagtcagtgaagggagcagtgtggcttgagtagtcaagagatgagagagaaactgggcagtagcagagatgacactggtgggtgttcaggagtatgttttaattctcccttctctcatagacacccactttccctcatcctctttctcctcaaggaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaaggtaaaatgggcccacatgacccaatccatgagcccaacaccccagcctttctaacaccactgtcttttgctccacttccctgtcactaaagcccctaaacttggtgccccatctctccacactgtctaaccccaacctctagaaatcaaggtttttctgtgtagggttgggttagcgtgttgttagagtaggggagtggattgagaaggaggctgaggggtactcaagggggctatagaatgtataggatttccctgaagcattcctagagagcctgcaaggtgaagatggctttggaaccagctggatctaggctgtgccacatactacctctttggccttggccacatccctaaactcttggattctgtttcctaagatgtaagatggaggtaattgttcctgcctcacaggagctgttgtgaggattaaacagagagtatgtctttagcgcggtgcctggcaccagtgcctggcatgtagtaggggcacaacaaatataaggtccactttgcttttcttttttctatagagaatcctttcctgtttgcattggaagccgtggttatctctgttggctccatgggattgattatcagccttctctgtgtgtatttctggctggaacggtgagatttggagaagcccagaaaaatgaggggaacggtagctgacaatagcagaggagggttttgcagggtctttaggagtaaaggatgagacagtaagtaatgagagattacccaagagggtttggtgatggaaggaagccacaggcacagagaacacagaatcactttatttcatatgggacaactgggagaagggtgataaaaaagctttaacctatgtgctcctgctccctctttctcccctgtcaggacgatgccccgaattcccaccctgaagaacctagaggatcttgttactgaataccacgggaacttttcggtgagaacgctgtcataagcatgctgcagtctatcaactgccaactgcctgccagcaagacagacagagtgtgggggtgggggcagagaggagagggaaggaggccctgcactaactgtcaggatgtggccgaccaaatggggcatggactatacagagagagacacacacagaagtgcagattatagattgaatgaggcagatggcaactggtattgggggcccaggagcctgtgtatcccttctgtaatcaattacagtggttgcagacatcatgagtactcctttggcacagagctcggtcttttacttcctgcccctaattgacccctgacctggacatatctgtctttaggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatc

SEQ ID NO: 4 - protein, 167 aa, Artificial Sequence
  Description of Artificial Sequence Synthetic I-CreI N75 scaffold protein sequence
MANTKYNKEFLLYLAGFVDGDGSIIAQIKPNQSYKFKHQLSLTFQVTQKTQRRWFLDKLVDEIGVGYVRDRGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 5 - DNA, 22 bp, Artificial Sequence
  Description of Artificial Sequence Synthetic IL2RG7 target sequence
ctcactatgttgcctaggctgg

SEQ ID NO: 6 - DNA, 22 bp, Artificial Sequence
  Description of Artificial Sequence Synthetic IL2RG4 target sequence
ctcactctgttgcccaggcttg

SEQ ID NO: 7 - DNA, 22 bp, Artificial Sequence
  Description of Artificial Sequence Synthetic IL2RG3 target sequence
cgacctctgtctccctggttca

SEQ ID NO: 8 - DNA, 22 bp, Artificial Sequence
  Description of Artificial Sequence Synthetic IL2RG5 target sequence
tggaacggtgagatttggagaa

SEQ ID NO: 9 - DNA, 22 bp, Artificial Sequence
  Description of Artificial Sequence Synthetic IL2RG6 target sequence
tcatatgggacaactgggagaa

SEQ ID NO: 10 - DNA, 24 bp, Artificial Sequence
  Description of Artificial Sequence Synthetic 5CTG_P target sequence
tcaaaacctggtaccaggttttga

SEQ ID NO: 11 - DNA, 24 bp, Artificial Sequence
  Description of Artificial Sequence Synthetic 5AGG_P target sequence
tcaaaacagggtaccctgttttga

SEQ ID NO: 12 - DNA, 22 bp, Artificial Sequence
  Description of Artificial Sequence Synthetic IL2RG3.2 target sequence
cgacctctggtaccctggttca

SEQ ID NO: 13 - DNA, 22 bp, Artificial Sequence
  Description of Artificial Sequence Synthetic IL2RG3.3 target sequence
cgacctctggtaccagaggtcg

SEQ ID NO: 14 - DNA, 22 bp, Artificial Sequence
  Description of Artificial Sequence Synthetic IL2RG3.4 target sequence
tgaaccagggtaccctggttca

SEQ ID NO: 15 - DNA, 24 bp, Artificial Sequence
  Description of Artificial Sequence Synthetic 10GAC_P target sequence
tcgacacgtcgtacgacgtgtcga

SEQ ID NO: 16 - DNA, 50 bp, Artificial Sequence
  Description of Artificial Sequence Synthetic oligo IL2RG3 sequence
tggcatacaagtttcgacctctggtaccagaggtcgacaatcgtctgtca

SEQ ID NO: 17 - DNA, 26 bp, Artificial Sequence
  Description of Artificial Sequence Synthetic GAL10F primer sequence
gcaactttagtgctgacacatacagg

SEQ ID NO: 18 - DNA, 24 bp, Artificial Sequence
  Description of Artificial Sequence Synthetic GAL10R primer sequence
acaaccttgattggagacttgacc

SEQ ID NO: 19 - DNA, 15 bp, Artificial Sequence
  Description of Artificial Sequence Synthetic assF primer sequence
ctannnttgaccttt

SEQ ID NO: 20 - DNA, 15 bp, Artificial Sequence
  Description of Artificial Sequence Synthetic assR primer sequence
aaaggtcaannntag

SEQ ID NO: 21 - DNA, 59 bp, Artificial Sequence
  Description of Artificial Sequence Synthetic PreATGCreFor primer sequence
gcataaattactatacttctatagacacgcaaacacaaatacacagcggccttgccacc

SEQ ID NO: 22 - DNA, 48 bp, Artificial Sequence
  Description of Artificial Sequence Synthetic I-CreIpostRev primer sequence
ggctcgaggagctcgtctagaggatcgctcgagttatcagtcggccgc

SEQ ID NO: 23 - DNA, 18 bp, Artificial Sequence
  Description of Artificial Sequence Synthetic AA78a83For primer sequence
ttaagcgaaatcaagccg

SEQ ID NO: 24 - DNA, 18 bp, Artificial Sequence
  Description of Artificial Sequence Synthetic AA78a83Rev primer sequence
cggcttgatttcgcttaa

SEQ ID NO: 25 - DNA, 33 bp, Artificial Sequence
  Description of Artificial Sequence Synthetic G19SF primer sequence
gccggctttgtggactctgacggtagcatcatc

SEQ ID NO: 26 - DNA, 33 bp, Artificial Sequence
  Description of Artificial Sequence Synthetic G19SR primer sequence
gatgatgctaccgtcagagtccacaaagccggc

SEQ ID NO: 27 - DNA, 33 bp, Artificial Sequence
  Description of Artificial Sequence Synthetic F54LF primer sequence
acccagcgccgttggctgctggacaaactagtg

SEQ ID NO: 28 - DNA, 33 bp, Artificial Sequence
  Description of Artificial Sequence Synthetic F54LR primer sequence
cactagtttgtccagcagccaacggcgctgggt

SEQ ID NO: 29 - DNA, 33 bp, Artificial Sequence
  Description of Artificial Sequence Synthetic F87LF primer sequence
aagccgctgcacaacctgctgactcaactgcag

SEQ ID NO: 30 - DNA, 33 bp, Artificial Sequence
  Description of Artificial Sequence Synthetic F87LR primer sequence
ctgcagttgagtcagcaggttgtgcagcggctt

SEQ ID NO: 31 - DNA, 33 bp, Artificial Sequence
  Description of Artificial Sequence Synthetic V105AF primer sequence
aaacaggcaaacctggctctgaaaattatcgaa

SEQ ID NO: 32 - DNA, 33 bp, Artificial Sequence
  Description of Artificial Sequence Synthetic V105AR primer sequence
ttcgataattttcagagccaggtttgcctgttt

SEQ ID NO: 33 - DNA, 33 bp, Artificial Sequence
  Description of Artificial Sequence Synthetic I132VF primer sequence
acctgggtggatcaggttgcagctctgaacgat

SEQ ID NO: 34 - DNA, 33 bp, Artificial Sequence
  Description of Artificial Sequence Synthetic I132VR primer sequence
atcgttcagagctgcaacctgatccacccaggt

SEQ ID NO: 35 - DNA, 77 bp, Artificial Sequence
  Description of Artificial Sequence Synthetic attB1-ICreIFor primer sequence
ggggacaagtttgtacaaaaaagcaggcttcgaaggagatagaaccatggccaataccaaatataacaaagagttcc

SEQ ID NO: 36 - DNA, 64 bp, Artificial Sequence
  Description of Artificial Sequence Synthetic attB1-ICreIRev primer sequence
ggggaccactttgtacaagaaagctgggtttagtcggccgccggggaggatttcttcttctcgc

SEQ ID NO: 37 - DNA, 18 bp, Artificial Sequence
  Description of Artificial Sequence Synthetic I-SceI target sequence
tagggataacagggtaat

SEQ ID NO: 38 - protein, 167 aa, Artificial Sequence
  Description of Artificial Sequence Synthetic first I-CreI variant /IL2RG7 sequence
MANTKYNKEFLLYLAGFVDGDGSIIAQIKPNQSTKFKHQLQLTFNVTQKTQRRWFLDKLVDEIGVGYVYDSGSVSYYQLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 39 - protein, 167 aa, Artificial Sequence
  Description of Artificial Sequence Synthetic first I-CreI variant /IL2RG4 sequence
MANTKYNKEFLLYLAGFVDGDGSIIAQIKPNQSTKFKHQLQLTFRVTQKTQRRWFLDKLVDEIGVGYVYDSGSVSEYYLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 40 - protein, 167 aa, Artificial Sequence
  Description of Artificial Sequence Synthetic first I-CreI variant /IL2RG3 (m10) sequence
MANTKYNKEFLLYLAGFVDGDGSIIAQIKPNQSRKFKHQLYLTFRVTQKTQRRWFLDKLVDEIGVGYVYDSGSVSDYTLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 41 - protein, 167 aa, Artificial Sequence
  Description of Artificial Sequence Synthetic first I-CreI variant /IL2RG5 sequence
MANTKYNKEFLLYLAGFVDGDGSIIAQIKPNQSRKFKHNLQLTFYVTQKTQRRWFLDKLVDEIGVGYVRDSGSVSDYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 42 - protein, 167 aa, Artificial Sequence
  Description of Artificial Sequence Synthetic first I-CreI variant /IL2RG6 sequence
MANTKYNKEFLLYLAGFVDGDGSIIAQIKPNQRDKFKHQLSLTFDVTQKTQRRWFLDKLVDEIGVGYVNDSGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 43 - protein, 167 aa, Artificial Sequence
  Description of Artificial Sequence Synthetic second  I-CreI variant /IL2RG7 sequence
MANTKYNKEFLLYLAGFVDGDGSIIAQISPNQSYKFKHRLKLTFDVTQKTQRRWFLDKLVDEIGVGYVNDSGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 44 - protein, 167 aa, Artificial Sequence
  Description of Artificial Sequence Synthetic second  I-CreI variant /IL2RG4 sequence
MANTKYNKEFLLYLAGFVDGDGSIIAQIKPNQTYKFKHQLSLTFDVTQKTQRRWFLDKLVDEIGVGYVYDSGSVSSYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 45 - protein, 167 aa, Artificial Sequence
  Description of Artificial Sequence Synthetic second I-CreI variant /IL2RG3 (M1) sequence
MANTKYNKEFLLYLAGFVDGDGSIIAQIKPRQTYKFKHQLSLTFAVTQKTQRRWFLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 46 - protein, 167 aa, Artificial Sequence
  Description of Artificial Sequence Synthetic second I-CreI variant /IL2RG5 sequence
MANTKYNKEFLLYLAGFVDGDGSIIAQIKPNQSTKFKHALSLTFQVTQKTQRRWFLDKLVDEIGVGYVYDSGSVSRYQLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 47 - protein, 167 aa, Artificial Sequence
  Description of Artificial Sequence Synthetic second I-CreI variant /IL2RG6 sequence
MANTKYNKEFLLYLAGFVDGDGSIIAQIKPNQSTKFKHALSLTFAVTQKTQRRWFLDKLVDEIGVGYVYDSGSVSYYKLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 48 - protein, 167 aa, Artificial Sequence
  Description of Artificial Sequence Synthetic I-CreI variant m1 sequence
MANTKYNKEFLLYLAGFVDGDGSIIAQIKPNQSHKFKHQLSLTFKVTQKTQRRWFLDKLVDEIGVGYVYDSGSVSEYQLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 49 - protein, 167 aa, Artificial Sequence
  Description of Artificial Sequence Synthetic I-CreI variant m3 sequence
MANTKYNKEFLLYLAGFVDGDGSIIAQIKPNQSHKFKHQLSLTFRVTQKTQRRWFLDKLVDEIGVGYVYDSGSVSDYQLSEIKPLHNFLTQLQPFLKLKQKQANLVLKIIEQLPSAKESPDKFLEVCTWVDQIAALNDSKTRKITSETVRAVLDSLSEKKKSSLAAD

SEQ ID NO: 50 - protein, 167 aa, Artificial Sequence
  Description of Artificial Sequence Synthetic I-CreI variant m4 sequence
MANTKYNKEFLLYLAGFVDGDGSIIAQITPNQSRKFKHQLRLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 51 - protein, 167 aa, Artificial Sequence
  Description of Artificial Sequence Synthetic I-CreI variant m5 sequence
MANTKYNKEFLLYLAGFVDGDGSIIAQIKPNQSRKFKHQLYLTFRVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 52 - protein, 167 aa, Artificial Sequence
  Description of Artificial Sequence Synthetic I-CreI variant m6 sequence
MANTKYNKEFLLYLAGFVDGDGSIIAQIKPNQSRKFKHQLQLTFKVTQKTQRRWFLDKLVDEIGVGYVYDSGSVSQYYLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 53 - protein, 167 aa, Artificial Sequence
  Description of Artificial Sequence Synthetic I-CreI variant m7 sequence
MANTKYNKEFLLYLAGFVDGDGSIIAQIKPNQSHKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 54 - protein, 167 aa, Artificial Sequence
  Description of Artificial Sequence Synthetic I-CreI variant m8 sequence
MANTKYNKEFLLYLAGFVDGDGSIIAQIKPNQSRKFKHQLRLTFRVTQKTQRRWFLDKLVDEIGVGYVYDSGSVSDYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 55 - protein, 167 aa, Artificial Sequence
  Description of Artificial Sequence Synthetic I-CreI variant m9 sequence
MANTKYNKEFLLYLAGFVDGDGSIIAQIKPNQSRKFKHQLRLTFRVTQKTQRRWFLDKLVDEIGVGYVYDSGSVSDYQLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 56 - protein, 167 aa, Artificial Sequence
  Description of Artificial Sequence Synthetic I-CreI variant m11 sequence
MANTKYNKEFLLYLAGFVDGDGSIIAQIKPNQSRKFKHQLALTFRVTQKTQRRWFLDKLVDEIGVGYVYDSGSVSDYQLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 57 - protein, 167 aa, Artificial Sequence
  Description of Artificial Sequence Synthetic I-CreI variant m12 sequence
MANTKYNKEFLLYLAGFVDGDGSIIAQIKPNQSRKFKHQLALTFRVTQKTQRRWFLDKLVDEIGVGYVYDSGSVSDYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 58 - protein, 167 aa, Artificial Sequence
  Description of Artificial Sequence Synthetic I-CreI variant m13 sequence
MANTKYNKEFLLYLAGFVDGDGSIIAQIKPNQSRKFKHQLALTFRVTQKTQRRWFLDKLVDEIGVGYVYDSGSVSEYVLSEIKPLHNFLTQLQPFLKLKQKQANLVLKIIEQLPSAKESPDKFLEVCTWVDQTAALNDSKTRKTTSETVRAVLDSLSEKKKSSPAAD

SEQ ID NO: 59 - protein, 167 aa, Artificial Sequence
  Description of Artificial Sequence Synthetic I-CreI variant m14 sequence
MANTKYNKEFLLYLAGFVDGDGSIIAQIKPNQSRKFKHQLSLTFRVTQKTQRRWFLDKLVDEIGVGYVYDSGSVSDYHLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 60 - protein, 167 aa, Artificial Sequence
  Description of Artificial Sequence Synthetic I-CreI variant m15 sequence
MANTKYNKEFLLYLAGFVDGDGSIIAQITPNQSPKFKHQLRLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 61 - protein, 167 aa, Artificial Sequence
  Description of Artificial Sequence Synthetic I-CreI variant m16 sequence
MANTKYNKEFLLYLAGFVDGDGSIIAQIKPNQSRKFKHQLYLTFRVTQKTQRRWFLDKLVDEIGVGYVYDSGSVSDYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 62 - protein, 167 aa, Artificial Sequence
  Description of Artificial Sequence Synthetic I-CreI variant m17 sequence
MANTKYNKEFLLYLAGFVDGDGSIIAQIKPNRSHKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 63 - protein, 167 aa, Artificial Sequence
  Description of Artificial Sequence Synthetic I-CreI variant m18 sequence
MANTKYNKEFLLYLAGFVDGDGSIIAQIKPNQSHKFKHQLSLTFKVTQKTQRRWFLDKLVDEIGVGYVYDSGSVSEYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 64 - protein, 167 aa, Artificial Sequence
  Description of Artificial Sequence Synthetic I-CreI variant m19 sequence
MANTKYNKEFLLYLAGFVDGDGSIIAQIKPNQSRKFKHQLYLTFRVTQKTQRRWFLDKLVDEIGVGYVYDSGSVSDYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 65 - protein, 167 aa, Artificial Sequence
  Description of Artificial Sequence Synthetic I-CreI variant m20 sequence
MANTKYNKEFLLYLAGFVDGDGSIIAQIKPNQSRKFKHQLALTFRVTQKTQRRWFLDKLVDEIGVGYVYDSGSVSDYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 66 - protein, 167 aa, Artificial Sequence
  Description of Artificial Sequence Synthetic I-CreI variant M2 sequence
MANTKYNKEFLLYLAGFVDGDGSIIAQIKPKQSCKFKHQLSLTFTVTQKTQRRWLLDKLVDEIGVGYVRDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 67 - protein, 167 aa, Artificial Sequence
  Description of Artificial Sequence Synthetic I-CreI variant IL2RG3.3 sequence
MANTKYNKEFLLYLAGFVDGDGSIIAQIKPNRSHKFKHQLSLTFKVTQKTQRRWFLDKLVDEIGVGYVYDSGSVSEYVLSGIKPLHNFLTQLQPFLKLKQKQANLVLKIIEQLPSAKESPDKFLEVCTWVDQIAALNDSKTRKTTSETVRAVLDGLSGKKKSSPAAD

SEQ ID NO: 68 - protein, 167 aa, Artificial Sequence
  Description of Artificial Sequence Synthetic I-CreI variant IL2RG3.3 sequence
MANTKYNKEFLLYLAGFVDGDGSIIAQIKPNRSHKFKHQLSLTFKVTQKTQRRWFLDKLVDEIGVGYVYDSRSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 69 - protein, 167 aa, Artificial Sequence
  Description of Artificial Sequence Synthetic I-CreI variant IL2RG3.3 sequence
MANTEYNKEFLLYLAGFVDGDGSIIAQIKPNQSRKFKHQIYLTFRVTQKTQRRWFLDKLVDEIGVGYVYDSGSVSDYTLSEIKPLHNLLTQLQPFLKLKQKQANLVLKIIEQLPSAKESPDKFLEVCTWVDQVAALNDSKTRKTTSETVRAVLDSLSEKKKSPPAAD

SEQ ID NO: 70 - protein, 167 aa, Artificial Sequence
  Description of Artificial Sequence Synthetic I-CreI variant IL2RG3.3 sequence
MANTKYNKEFLLYLAGFVDGDGSIIAQIKPNRSHKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSRTRKTTSETVRAVLDSLSEKKKSSPAAD

SEQ ID NO: 71 - protein, 167 aa, Artificial Sequence
  Description of Artificial Sequence Synthetic I-CreI variant IL2RG3.3 sequence
MANTKYNKEFLLYLAGFVDSDGSIIAQIKPNQSHKFKHQLYLTFRVTQKTQRRWFLDKLVDEIGVGYVYDSGSVSDYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 72 - protein, 167 aa, Artificial Sequence
  Description of Artificial Sequence Synthetic I-CreI variant IL2RG3.3 sequence
MANTKYNKEFLLYLAGFVDGDGSIIARIKPNRSHKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 73 - protein, 167 aa, Artificial Sequence
  Description of Artificial Sequence Synthetic I-CreI variant .3_R1 sequence
MANTKYNKEFLLYLAGFVDGDGSIIARIKPNRSHKFKHQLSLTFKVTQKTQRRWFLDKLVDEIGVGYVYDSGSVSEYVLSEIKPLHNFLAQLQPFLKLKQKQANLVLKIIEQLPSAKGSPDKFLEVCTWVDQIAALNDSRTRKTTSETVRAVLDSLSEKKKSSPAAD

SEQ ID NO: 74 - protein, 167 aa, Artificial Sequence
  Description of Artificial Sequence Synthetic I-CreI variant .3_R2 sequence
MANTKYNKEFLLYLAGFVDGDGSIIARIKPNRSHKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSRTRKTTSETVRAVLDSLSEKKKSSPAAD

SEQ ID NO: 75 - protein, 167 aa, Artificial Sequence
  Description of Artificial Sequence Synthetic I-CreI variant .3_R3 sequence
MANTKYNKEFLLYLAGFVDGDGSIIARIKPNRSHKFKHQISLTFKVTQKTQRRWFLDKLVDEIGVGYVYDSGSVSEYVLSEIRPLHNFLTQLQPFLKLKQKQANLVLKIIEQLPSAKESPDKFLEVCTWVDQIAALNDSRTRKTTSETVRAVLDSLSEKKKSSPAAD

SEQ ID NO: 76 - protein, 167 aa, Artificial Sequence
  Description of Artificial Sequence Synthetic I-CreI variant .3_R4 sequence
MANTKYNKEFLLYLAGFVDGDGSIIARIKPNRSHKFKHQISLTFKVAQKTQRRWFLDKLVDEIGVGYVYDSRSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 77 - protein, 167 aa, Artificial Sequence
  Description of Artificial Sequence Synthetic I-CreI variant .3_R5 sequence
MANTKYNKEFLLYLAGFVDGDGSIIARIKPNRSHKFKHQIYLTFRVTQKTQRRWFLDKLVDEIGVGYVYDSGSVSDYTLSEIKPLHNLLTQLQPFLKLKQKQANLVLKIIEQLPSAKESPDKFLEVCTWVDQVAALNDSKTRKTTSETVRAVLDSLSEKKKSPPAAD

SEQ ID NO: 78 - protein, 167 aa, Artificial Sequence
  Description of Artificial Sequence Synthetic I-CreI variant .3_R6 sequence
MANTKYNEEFLLYLAGFVDGDGSIIARIKPNRSHKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSRTRKTTSETVRAVLDSLSEKKKSSPAAD

SEQ ID NO: 79 - protein, 167 aa, Artificial Sequence
  Description of Artificial Sequence Synthetic I-CreI variant .3_R7 sequence
MANTKYNKEFLLYLAGFVDGDGSIIARIKPNRSHKFKHQLSLTFKVTQKTQRRWFLDKLVDEIGVGYVYDSGSVSEYVLSEIKPLHNFLTQLQPFLKLKQKQANLVLKIIERLPSAKESPDKFLEVCTWVDQIAALNDSRTRKTTSETVRAVLDSLSEKKKSSPAAD

SEQ ID NO: 80 - protein, 167 aa, Artificial Sequence
  Description of Artificial Sequence Synthetic I-CreI variant .3_R8 sequence
MADTKYNKEFLLYLAGFVDGDGSIIARIKPNRSHKFKHQLSLTFKVTQKTQRRWFLDKLVDEIGVGYVYDSGSVSEYVLSGIKPLHNFLTQLQPFLKLKQKQANLVLKIIEQLPSAKESPDRFLEVCTWVDQIAALNDSRTRKTTSETVRAVLDSLSEKKKSSPAAD

SEQ ID NO: 81 - protein, 167 aa, Artificial Sequence
  Description of Artificial Sequence Synthetic I-CreI variant .3_R9 sequence
MANTKYNKEFLLYLAGFVDGDGSIIARIKPNRSHKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSRTRKTTSETVRAVLDSLSEKRKSSPAAD

SEQ ID NO: 82 - protein, 167 aa, Artificial Sequence
  Description of Artificial Sequence Synthetic I-CreI variant .3_R10 sequence
MANTKYNKEFLLYLAGFVDSDGSIIAQIKPNQSHKFKHQLYLTLRVTQKTQRRWFLDKLVDEIGVGYVYDSGSVSDYTLSEIKPLHNFLTQLQPFLKLKQKQANLVLKIIEQLPSAKESPDKFLEVCTWVDQVAALNDSKTRKTTSETVRAVLDSLSEKEGSFPAAD

SEQ ID NO: 83 - protein, 167 aa, Artificial Sequence
  Description of Artificial Sequence Synthetic I-CreI variant .3_R11 sequence
MANTKYNKEFLLYLAGFVDSDGSIIAQIKPNQSHKFKHQLYLTFKVTQKTQRRWFLDKLVDEIGVGYVYDSRSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 84 - protein, 167 aa, Artificial Sequence
  Description of Artificial Sequence Synthetic I-CreI variant .3_R12 sequence
MANTKYNKEFLLYLAGFVDSDGSIIARIKPNRSHKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 85 - protein, 167 aa, Artificial Sequence
  Description of Artificial Sequence Synthetic I-CreI variant .3_R13 sequence
MANTKYNKEFLLYLAGFVDSDGSIIAQIKPNRSHKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSRTRKTTSETVRAVLDSLSEKKKSSPAAD

SEQ ID NO: 86 - protein, 167 aa, Artificial Sequence
  Description of Artificial Sequence Synthetic I-CreI variant .3_R14 sequence
MANTKYNKEFLLYLAGFVDSDGSIIAQIKPNQSHKFKHQLYLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSRTRKTTSETVRAVLDSLSEKKKSSPAAD

SEQ ID NO: 87 - protein, 167 aa, Artificial Sequence
  Description of Artificial Sequence Synthetic I-CreI variant .3_R15 sequence
MANTKYNKGFLLYLAGFVDSDGSIIARIKPNRSHKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSRTRKTTSETVRAVLDSLSEKKKSSPAAD

SEQ ID NO: 88 - protein, 167 aa, Artificial Sequence
  Description of Artificial Sequence Synthetic I-CreI variant .3_R16 sequence
MANTKYNKEFLLYLAGFVDSDGSIIAQIKPNQSRKFKHQLYLTFRVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 89 - protein, 167 aa, Artificial Sequence
  Description of Artificial Sequence Synthetic I-CreI variant .3_R17 sequence
MANTKYNKEFLLYLAGFVDGDGSIIARIKPNRSHKFKHQLSLTFKVTQKTQRRWLLDKLVDEIGVGYVYDSGSVSEYVLSEIKPLHNFLTQLQPFLKLKQKQANLVLKIIEQLPSAKESPDKFLEVCTWVDQIAALNDSRTRKTTSETVRAVLDSLSEKKKSSPAAD

SEQ ID NO: 90 - protein, 167 aa, Artificial Sequence
  Description of Artificial Sequence Synthetic I-CreI variant .3_R18 sequence
MANTKYNKEFLLYLAGFVDGDGSIIAQIKPNRSHKFKHQLSLTFKVTQKTQRRWFLDKLVDEIGVGYVYDSRSVSEYVLSEIKPLHNLLTQLQPFLKLKQKQANLVLKIIEQLPSAKESPDKFLEVCTWVDQVAALNDSRTRKTTSEAVRAVLDSLSEKKKSSPAAD

SEQ ID NO: 91 - protein, 167 aa, Artificial Sequence
  Description of Artificial Sequence Synthetic I-CreI variant .3_R19 sequence
MANTKYNKEFLLYLAGFVDSDGSIIAQIKPNQSHKFKHQLYLTFRVTQKTQRRWFLDKLVDEIGVGYVYDSGSVSDYTLSEIKPLHNLLTQLQPFLKLKQKQANLVLKIIEQLPSAKESPDKFLEVCTWVDQIAALNDSRTRKTTSETVRAVLDSLSEKKKSSPAAD

SEQ ID NO: 92 - protein, 167 aa, Artificial Sequence
  Description of Artificial Sequence Synthetic I-CreI variant .3_R20 sequence
MANTKYNKEFLLYLAGFVDGDGSIIAQIKPNQSRKFKHQLYLTFRVTQKTQRRWFLDKLVDEIGVGYVYDSGSVSDYTLSEIKPLHNLLTQLQPFLKLKQKQANLVLKIIEQLPSAKESPDKFLEVCTWVDQIAALNDSKTRKTTSETVRAVLDSLSEKKKSSPAAD

SEQ ID NO: 93 - protein, 167 aa, Artificial Sequence
  Description of Artificial Sequence Synthetic I-CreI variant .3_R21 sequence
MANTKYNKEFLLYLAGFVDSDGSIIAQIKPNQSHKFKHQLYLTFRVTQKTQRRWFLDKLVDEIGVGYVYDSGSVSDYTLSEIKPLHNLLTQLQPFLKLKQKQANLVLKIIEQLPSAKESPDKFLEVCTWVDQIAALNDSKTRKTTSETVRAVLDGLSGKKKSSPAAD

SEQ ID NO: 94 - protein, 167 aa, Artificial Sequence
  Description of Artificial Sequence Synthetic I-CreI variant .3_R22 sequence
MANTKYNKEFLLYLAGFVDGDGSIIAQIKPNRSHKFKHQLSLTFKVTQKTQRRWFLDKLVDEIGVGYVYDSGSVSEYVLSGIKPLHNFLTQLQPFLKLKQKQANLALKIIEQLPSAKESPDKFLEVCTWVDQIAALNDSRTRKTTSETVRAVLDSLSEKKKSSPAAD

SEQ ID NO: 95 - protein, 167 aa, Artificial Sequence
  Description of Artificial Sequence Synthetic I-CreI variant .3_R23 sequence
MANTKYNKEFLLYLAGFVDSDGSIIAQIKPNQSHKFKHQLYLTFRVTQKTQRRWFLDKLVDEIGVGYVYDSGSVSDYTLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 96 - protein, 167 aa, Artificial Sequence
  Description of Artificial Sequence Synthetic I-CreI variant .3_R24 sequence
MANTKYNKEFLLYLAGFVDSDGSIIAQIKPNQSHKFKHQLYLTFRVTQKTQRRWFLDKLVDEIGVGYVYDSGSVSDYTLSEIKPLHNFLTQLQPFLKLKQKQANLVLKIIEQLPSAKESPDKFLEVCTWVDQVAALNDSKTRKTTSETVRAVLDGLSEKKKSSPAAD

SEQ ID NO: 97 - protein, 167 aa, Artificial Sequence
  Description of Artificial Sequence Synthetic I-CreI variant .3_R25 sequence
MANTKYNKEFLLYLAGFVDSDGSIIAQIKPNQSHKFKHQLYLTFRVTQKTQRRWFLDKLVDEIGVGYVYDSGSVSEYVLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 98 - protein, 167 aa, Artificial Sequence
  Description of Artificial Sequence Synthetic I-CreI variant .3_R26 sequence
MANTKYNKEFLLYLAGFVDGDGSIIAQIKPNRSHKFKHQLSLTFKVTQKTQRRWFLDKLVDEIGVGYVYDSRSVSEYVLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 99 - protein, 167 aa, Artificial Sequence
  Description of Artificial Sequence Synthetic I-CreI variant .3_R27 sequence
MANTKYNKEFLLYLAGFVDGDGSIIAQIKPNRSHKFKHQLSLTFKVTQKTQRRWFLDKLVDEIGVGYVYDSGSVSEYVLSGIKPLHNFLTQLQPFLKLKQKQANLVLKIIEQLPSAKESPDKFLEVCTWVDQVAALNDSRTRKTTSETVRAVLDSLSEKKKSSPAAD

SEQ ID NO: 100 - protein, 167 aa, Artificial Sequence
  Description of Artificial Sequence Synthetic I-CreI variant .3_R28 sequence
MANTKYNKEFLLYLAGFVDGDGSIIAQIKPNRSHKFKHQLSLTFKVTQKTQRRWFLDKLVDEIGVGYVYDSGSVSEYVLSEIKPLHNFLTQLQPFLKLKQKQANLVLKIIEQLPSAKESPDKFLEVCTWVDQVAALNDSRTRKTTSETVRAVLDSLSEKKKSSPAAD

SEQ ID NO: 101 - protein, 167 aa, Artificial Sequence
  Description of Artificial Sequence Synthetic I-CreI variant .4_R0 sequence
MANTKYNKEFLLYLAGFVDGDGSIIAQIKPRQTYKFKHQLSLTFAVTQKTQRRWFLDKLVDEIGVGYVYDSGSVSEYRLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 102 - protein, 167 aa, Artificial Sequence
  Description of Artificial Sequence Synthetic I-CreI variant .4_R1 sequence
MANTKYNKEFLLYLAGFVDSDGSIIAQIKPRQTYKFKHQLSLTFAVTQKTQRRWFLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 103 - protein, 167 aa, Artificial Sequence
  Description of Artificial Sequence Synthetic I-CreI variant .4_R2 sequence
MANTKYNKEFLLYLAGFVDSDGSIIAQIKPRQTYKFKHQLSLTFAVTQKTQRRWFLDKLADEIGVGYVYDSGSVSEYRLSEIKPLHNFLTQLQPFLKLKQKQANLVLKIIERLPSAKESPDKFLEVCTWVDQIAALNDSKTRKTTSETVRAVLDSLSEKKKSSPAAD

SEQ ID NO: 104 - protein, 167 aa, Artificial Sequence
  Description of Artificial Sequence Synthetic I-CreI variant .4_R3 sequence
MANTKYNKEFLLYLAGFVDSDGSIIAQIKPRQTYKFKHQLSLTFAVTQKTQRRWFLDKLVGEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 105 - protein, 167 aa, Artificial Sequence
  Description of Artificial Sequence Synthetic I-CreI variant .4_R4 sequence
MANTKYNKEFLLYLAGFVDSDGSIIAQIKPRQTYKFKHQLSLTFAVTQKTQRRWFLDKLADEIGVGYVYDSGSVSEYRLSEIKPLHNFLTQLQPFLKLKQKQANLVLKIIERLPSAKESPDKYLEVCTWVDQIAALNDSKTRKTTSETVRAVLDSLSEKKKSSPAAD

SEQ ID NO: 106 - protein, 167 aa, Artificial Sequence
  Description of Artificial Sequence Synthetic I-CreI variant .4_R5 sequence
MANTKYNKEFLLYLAGFVDSDGSIIAQIKPRQTYKFKHQLSLTFAVTQKTQRRWFLDKLADEIGVGYVYDSGSVSEYRLSEIKPLHNFLTQLQYFLKRKQKQAYLVLRIIEQLPSAKESPDKFLEVCTWVDQIAALNDSKTRKTTSETVRAVLDSLSEKKKSSPAAD

SEQ ID NO: 107 - protein, 167 aa, Artificial Sequence
  Description of Artificial Sequence Synthetic I-CreI variant .4_R6 sequence
MANTKYNKEFLLYLAGFVDSDGSIIAQIKPRQTYKFKHQLSLTFAVTQKTQRRWFLDKLVGEIGVGYVYDSGSVSEYRLSEIKPLHNFLTQLQPFLRLRQKQANLVLKIIEQLPSAKESPDKFLEVCTWVDQIAALNDSKTRKTTSETVRAVLDSLSEKKKSSPAAD

SEQ ID NO: 108 - protein, 167 aa, Artificial Sequence
  Description of Artificial Sequence Synthetic I-CreI variant .4_R8 sequence
MANTKYNKEFLLYLAGFVDSDGSIIAQIKPRQTYKFKHQLSLTFAVTQKTQRRWFLDKLVGEIGVGYVYDSGSVSEYRLSEIKPLHNFLTQLQPFLKLKQKQANLVLKIIEQLPSAKESPDKFLEVCTWVDQIAAQNDSKTRKTTSETVRAVLGSLSEKKKSSPGPD

SEQ ID NO: 109 - protein, 167 aa, Artificial Sequence
  Description of Artificial Sequence Synthetic I-CreI variant .4_R9 sequence
MANTKYNKEFLLYLAGFVDSDGSIIAQIKPRQTYKFKHQLSLTFAVTQKTQRRWFLDKLVGEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLREKKKSSPAAD

SEQ ID NO: 110 - protein, 167 aa, Artificial Sequence
  Description of Artificial Sequence Synthetic I-CreI variant .4_R10 sequence
MANTKYNKEFLLYLAGFVDSDGSIIAQIKPRQTYKFKHQLSLTFAVTQKTQRCWFLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 111 - protein, 167 aa, Artificial Sequence
  Description of Artificial Sequence Synthetic I-CreI variant .4_R11 sequence
MANTKYNKEFLLYLAGFVDSDGSIIAQIKPRQTYKFKHQLSLTFAVTQKTQRRWFLDKLADEIGVGYVYDSGSVSEYRLSEIRPLHNFLTQLQPFLKLKQKQANLVLKIIERLPSAKESPDKFLEVCTWVDQIAALNDSKARKTTSETVRAVLDSLSEKKKSSPAAD

SEQ ID NO: 112 - DNA, 1451 bp, Homo sapiens
  CDS (15)..(1121)
gaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatc

SEQ ID NO: 113 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 114 - DNA, 24 bp, Artificial Sequence
  Description of Artificial Sequence Synthetic 10GAA_P target sequence
tcgaaacgtcgtacgacgtttcga

SEQ ID NO: 115 - protein, 167 aa, Artificial Sequence
  Description of Artificial Sequence Synthetic I-CreI variant m2 sequence
MANTKYNKEFLLYLAGFVDGDGSIIAQIKPNQSRKFKHQLSLTFRVTQKTQRRWFLDKLVDEIGVGYVYDSGSVSDYQLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 116 - DNA, 22 bp, Artificial Sequence
  Description of Artificial Sequence Synthetic IL2RG13 DNA target sequence
agggatactgtgggacattgga

SEQ ID NO: 117 - DNA, 22 bp, Artificial Sequence
  Description of Artificial Sequence Synthetic IL2RG14 DNA target sequence
gatcctgacttgtctaggccag

SEQ ID NO: 118 - DNA, 22 bp, Artificial Sequence
  Description of Artificial Sequence Synthetic IL2RG12 DNA target sequence
ttgcctagtgtggatgggcaga

SEQ ID NO: 119 - DNA, 22 bp, Artificial Sequence
  Description of Artificial Sequence Synthetic IL2RG15 DNA target sequence
gaagcccagaaaaatgagggga

SEQ ID NO: 120 - protein, 167 aa, Artificial Sequence
  Description of Artificial Sequence Synthetic first I-CreI variant/IL2RG13 target sequence
MANTKYNKEFLLYLAGFVDGDGSIIAQIKPHQTCKFKHRLSLTFQVTQKTQRRWFLDKLVDEIGVGYVRDDGSVSNYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 121 - protein, 167 aa, Artificial Sequence
  Description of Artificial Sequence Synthetic second I-CreI variant/IL2RG13 target sequence
MANTKYNKEFLLYLAGFVDGDGSIIAQIKPNQDYKFKHYLSLTFQVTQKTQRRWFLDKLVDEIGVGYVRDSGSVSHYYLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 122 - protein, 167 aa, Artificial Sequence
  Description of Artificial Sequence Synthetic first I-CreI variant/IL2RG14 target sequence
MANTKYNKEFLLYLAGFVDGDGSIIAQIKPRQANKFKHQLELTFYVTQKTQRRWFLDKLVDEIGVGYVRDSGSVSQYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 123 - protein, 167 aa, Artificial Sequence
  Description of Artificial Sequence Synthetic second I-CreI variant/IL2RG14 target sequence
MANTKYNKEFLLYLAGFVDGDGSIIAQIKPNQSCKFKHALSLTFNVTQKTQRRWFLDKLVDEIGVGYVRDSGSVSYYNLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 124 - protein, 167 aa, Artificial Sequence
  Description of Artificial Sequence Synthetic first I-CreI variant/IL2RG12 target sequence
MANTKYNKEFLLYLAGFVDGDGSIIAQIKPNQSSKFKHRLELTFLVTQKTQRRWFLDKLVDEIGVGYVRDNGSVSNYILSKIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 125 - protein, 167 aa, Artificial Sequence
  Description of Artificial Sequence Synthetic second I-CreI variant/IL2RG12 target sequence
MANTKYNKEFLLYLAGFVDGDGSIIAQIKPDQSRKFKHTLSLTFAVTQKTQRRWFLDKLVDEIGVGYVYDSGSVSYYKLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 126 - protein, 167 aa, Artificial Sequence
  Description of Artificial Sequence Synthetic first I-CreI variant/IL2RG15 target sequence
MANTKYNKEFLLYLAGFVDGDGSIIAQIKPSQSCKFKHQLALTFAVTQKTQRRWFLDKLVDEIGVGYVYDSGSVSYYKLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 127 - protein, 167 aa, Artificial Sequence
  Description of Artificial Sequence Synthetic second  I-CreI variant/IL2RG15 target sequence
MANTKYNKEFLLYLAGFVDGDGSIIAQIKPNQTYKFKHWLSLTFAVTQKTQRRWFLDKLVDEIGVGYVYDSGSVSYYKLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 128 - protein, 167 aa, Artificial Sequence
  Description of Artificial Sequence Synthetic IL2RG3.4 variant M1_24V sequence
MANTKYNKEFLLYLAGFVDGDGSIVAQIKPRQTYKFKHQLSLTFAVTQKTQRRWFLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 129 - protein, 167 aa, Artificial Sequence
  Description of Artificial Sequence Synthetic IL2RG3.4 variant M1_24T sequence
MANTKYNKEFLLYLAGFVDGDGSITAQIKPRQTYKFKHQLSLTFAVTQKTQRRWFLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 130 - protein, 167 aa, Artificial Sequence
  Description of Artificial Sequence Synthetic IL2RG3.4 variant M1_34R sequence
MANTKYNKEFLLYLAGFVDGDGSIIAQIKPRQTYRFKHQLSLTFAVTQKTQRRWFLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 131 - protein, 167 aa, Artificial Sequence
  Description of Artificial Sequence Synthetic IL2RG3.4 variant M1_43I sequence
MANTKYNKEFLLYLAGFVDGDGSIIAQIKPRQTYKFKHQLSLTIAVTQKTQRRWFLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 132 - protein, 167 aa, Artificial Sequence
  Description of Artificial Sequence Synthetic IL2RG3.4 variant M1_64A sequence
MANTKYNKEFLLYLAGFVDGDGSIIAQIKPRQTYKFKHQLSLTFAVTQKTQRRWFLDKLVDEIGAGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 133 - protein, 167 aa, Artificial Sequence
  Description of Artificial Sequence Synthetic IL2RG3.4 variant M1_100R sequence
MANTKYNKEFLLYLAGFVDGDGSIIAQIKPRQTYKFKHQLSLTFAVTQKTQRRWFLDKLVDEIGVGYVYDSGSVSEYRLSEIKPLHNFLTQLQPFLKLKQRQANLVLKIIEQLPSAKESPDKFLEVCTWVDQIAALNDSKTRKTTSETVRAVLDSLSEKKKSSPAAD

SEQ ID NO: 134 - protein, 167 aa, Artificial Sequence; identical to SEQ ID NO: 101
  Description of Artificial Sequence Synthetic IL2RG3.4 variant M1_132V sequence

SEQ ID NO: 135 - protein, 167 aa, Artificial Sequence
  Description of Artificial Sequence Synthetic IL2RG3.4 variant M1_43L sequence
MANTKYNKEFLLYLAGFVDGDGSIIAQIKPRQTYKFKHQLSLTLAVTQKTQRRWFLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 136 - protein, 167 aa, Artificial Sequence
  Description of Artificial Sequence Synthetic IL2RG3.4 variant M1_31R_34R sequence
MANTKYNKEFLLYLAGFVDGDGSIIAQIKPRRTYRFKHQLSLTFAVTQKTQRRWFLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 137 - protein, 167 aa, Artificial Sequence
  Description of Artificial Sequence Synthetic IL2RG3.4 variant M1_57R_107E sequence
MANTKYNKEFLLYLAGFVDGDGSIIAQIKPRQTYKFKHQLSLTFAVTQKTQRRWFLDRLVDEIGVGYVYDSGSVSEYRLSEIKPLHNFLTQLQPFLKLKQKQANLVLEIIEQLPSAKESPDKFLEVCTWVDQIAALNDSKTRKTTSETVRAVLDSLSEKKKSSPAAD

SEQ ID NO: 138 - protein, 167 aa, Artificial Sequence
  Description of Artificial Sequence Synthetic IL2RG3.4 variant M1_103D sequence
MANTKYNKEFLLYLAGFVDGDGSIIAQIKPRQTYKFKHQLSLTFAVTQKTQRRWFLDKLVDEIGVGYVYDSGSVSEYRLSEIKPLHNFLTQLQPFLKLKQKQADLVLKIIEQLPSAKESPDKFLEVCTWVDQIAALNDSKTRKTTSETVRAVLDSLSEKKKSSPAAD

SEQ ID NO: 139 - protein, 167 aa, Artificial Sequence
  Description of Artificial Sequence Synthetic IL2RG3.4 variant M1_117K sequence
MANTKYNKEFLLYLAGFVDGDGSIIAQIKPRQTYKFKHQLSLTFAVTQKTQRRWFLDKLVDEIGVGYVYDSGSVSEYRLSEIKPLHNFLTQLQPFLKLKQKQANLVLKIIEQLPSAKKSPDKFLEVCTWVDQIAALNDSKTRKTTSETVRAVLDSLSEKKKSSPAAD

SEQ ID NO: 140 - protein, 167 aa, Artificial Sequence
  Description of Artificial Sequence Synthetic ILRG3.3 variant .3R_25a sequence
MANTKYNKEFLLYLAGFVDSDGSIIAQIKPNQSHKFKHQLYLTFRVTQKTQRRWFLDKLVDEIGVGYVYDSRSVSEYVLSEIKPLHNFLTQLQPFLKLKQKQANLVLKIIEQLPSAKESPDKFLEVCTWVDQVAALNDSRTRKTTSETVRAVLDSLSEKKKSSPAAD

SEQ ID NO: 141 - protein, 167 aa, Artificial Sequence
  Description of Artificial Sequence Synthetic ILRG3.3 variant .3R_25b sequence
MANTKYNKEFLLYLAGFVDSDGSIIAQIKPNQSHKFKHQLYLTFRVTQKTQRRWFLDKLVDEIGVGYVYDSGSVSDYTLSEIKPLHNFLTQLQPFLKLKQKQANLVLKIIEQLPSAKESPDKFLEVCNWVDQVAALNDSKTRKTTSETVRAVLDSLSEKKKSSPAAD

SEQ ID NO: 142 - protein, 167 aa, Artificial Sequence
  Description of Artificial Sequence Synthetic ILRG3.3 variant .3R_25c sequence
MANTKYNKEFLLYLAGFVDSDGSIIAQIKPNQSHKFKHQLYLTFRVTQKTQRRWFLDKLVDEIGVGYVYDSRSVSEYVLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 143 - protein, 167 aa, Artificial Sequence
  Description of Artificial Sequence Synthetic IL2RG3.4 variant M1_24V_132V sequence
MANTKYNKEFLLYLAGFVDGDGSIVAQIKPRQTYKFKHQLSLTFAVTQKTQRRWFLDKLVDEIGVGYVYDSGSVSEYRLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 144 - protein, 167 aa, Artificial Sequence
  Description of Artificial Sequence Synthetic IL2RG3.4 variant M1_24V_80K sequence
MANTKYNKEFLLYLAGFVDGDGSIVAQIKPRQTYKFKHQLSLTFAVTQKTQRRWFLDKLVDEIGVGYVYDSGSVSEYRLSKIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 145 - protein, 167 aa, Artificial Sequence
  Description of Artificial Sequence Synthetic IL2RG3.4 variant M1_24V_54L sequence
MANTKYNKEFLLYLAGFVDGDGSIVAQIKPRQTYKFKHQLSLTFAVTQKTQRRWLLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 146 - protein, 167 aa, Artificial Sequence
  Description of Artificial Sequence Synthetic IL2RG3.4 variant M1_24V_87L sequence
MANTKYNKEFLLYLAGFVDGDGSIVAQIKPRQTYKFKHQLSLTFAVTQKTQRRWFLDKLVDEIGVGYVYDSGSVSEYRLSEIKPLHNLLTQLQPFLKLKQKQANLVLKIIEQLPSAKESPDKFLEVCTWVDQIAALNDSKTRKTTSETVRAVLDSLSEKKKSSPAAD

SEQ ID NO: 147 - protein, 167 aa, Artificial Sequence
  Description of Artificial Sequence Synthetic IL2RG3.4 variant M1_24V_105A sequence
MANTKYNKEFLLYLAGFVDGDGSIVAQIKPRQTYKFKHQLSLTFAVTQKTQRRWFLDKLVDEIGVGYVYDSGSVSEYRLSEIKPLHNFLTQLQPFLKLKQKQANLALKIIEQLPSAKESPDKFLEVCTWVDQIAALNDSKTRKTTSETVRAVLDSLSEKKKSSPAAD

SEQ ID NO: 148 - protein, 167 aa, Artificial Sequence
  Description of Artificial Sequence Synthetic IL2RG3.4 variant M1_24V_105A_132V sequence
MANTKYNKEFLLYLAGFVDGDGSIVAQIKPRQTYKFKHQLSLTFAVTQKTQRRWFLDKLVDEIGVGYVYDSGSVSEYRLSEIKPLHNFLTQLQPFLKLKQKQANLALKIIEQLPSAKESPDKFLEVCTWVDQVAALNDSKTRKTTSETVRAVLDSLSEKKKSSPAAD

SEQ ID NO: 149 - DNA, 22 bp, Artificial Sequence
  Description of Artificial Sequence Synthetic IL2C_P target sequence
taaaccagggtaccctggttta

SEQ ID NO: 150 - DNA, 22 bp, Artificial Sequence
  Description of Artificial Sequence Synthetic IL2RG3.6 DNA target sequence
tgaaccaggtctccctggttca

SEQ ID NO: 151 - DNA, 33 bp, Artificial Sequence
  Description of Artificial Sequence Synthetic E80KF primer sequence
ttaagcaaaatcaagccgctgcacaacttcctg

SEQ ID NO: 152 - DNA, 33 bp, Artificial Sequence
  Description of Artificial Sequence Synthetic E80KR primer sequence
caggaagttgtgcagcggcttgattttgcttaa

SEQ ID NO: 153 - DNA, 30 bp, Artificial Sequence
  Description of Artificial Sequence Synthetic 10RG34For primer sequence
aagtttaaacatcagctaagcttgaccttt

SEQ ID NO: 154 - DNA, 51 bp, Artificial Sequence
  Description of Artificial Sequence Synthetic 10RG34Rev1 primer sequence
caagcttagctgatgtttaaacttmnnagactgmnntggtttaatctgagc

SEQ ID NO: 155 - DNA, 51 bp, Artificial Sequence
  Description of Artificial Sequence Synthetic 10RG34Rev2 primer sequence
caagcttagctgatgtttaaacttmbnmbnctggtttggmbnaatctgagc

SEQ ID NO: 156 - protein, 167 aa, Artificial Sequence
  Description of Artificial Sequence Synthetic IL2RG3.4 variant Seq1 sequence
MANTKYNKEFLLYLAGFVDGDGSIVAQIKPRQSYKFKHQLSLTFTVTQKTQRRWFLDKLVDEIGVGYVRDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 157 - protein, 167 aa, Artificial Sequence
  Description of Artificial Sequence Synthetic IL2RG3.4 variant Seq2 sequence
MANTKYNKEFLLYLAGFVDGDGSIVAQIKPRQSNKFKHQLSLTFTVTQKTQRRWFLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 158 - protein, 167 aa, Artificial Sequence
  Description of Artificial Sequence Synthetic IL2RG3.4 variant Seq3 sequence
MANTKYNKEFLLYLAGFVDGDGSIVAQIKPRQSAKFKHQLSLTFTVTQKTQRRWFLDKLVDEIGVGYVRDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 159 - protein, 167 aa, Artificial Sequence
  Description of Artificial Sequence Synthetic IL2RG3.4 variant Seq4 sequence
MANTKYNKEFLLYLAGFVDGDGSIVAQIKPRQSVKFKHQLSLTFTVTQKTRRRWFLDKLVDEIGVGYVRDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 160 - protein, 167 aa, Artificial Sequence
  Description of Artificial Sequence Synthetic IL2RG3.4 variant Seq5 sequence
MANTKYNKEFLLYLAGFVDGDGSIVAQIKPRQSSKFKHQLSLTFRVTQKTQRRWFLDKLVDEIGVGYVYDSGSVSEYTLSEIKPLHNFLTQLQPFLKLKQKQANLVLKIIEQLPSAKESPDKFLEVCTWADQIAALNDSKTRKTTSETVRAVLDSLSEKKKSSPAAD

SEQ ID NO: 161 - protein, 167 aa, Artificial Sequence
  Description of Artificial Sequence Synthetic IL2RG3.4 variant Seq6 sequence
MANTKYNKEFLLYLAGFVDGDGSIVAQIKPRQSVKFKHQLSLTFTVTQKTQRRWFLDKLVDEIGVGYVYDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 162 - protein, 167 aa, Artificial Sequence
  Description of Artificial Sequence Synthetic IL2RG3.4 variant Seq7 sequence
MANTKYNKEFLLYLAGFVDGDGSIVAQIKPNQGHKFKHQLSLTFTVTQKTQRRWFLDKLVDEIGVGYVRDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 163 - protein, 167 aa, Artificial Sequence
  Description of Artificial Sequence Synthetic IL2RG3.6 variant Amel1 sequence
MANTKYNKEFLLYLAGFVDGDGSIVAQIKPNQGHKFKHQLSLTFTVTQKTRRRWFLDKLVDEIGVGYVRDSGSVSEYRLSEIKPLHNFLTQLQPFLKLKQKQANLVLKIIEQLPSAKESPDKFLEVCTWVDQVAALNDSKTRKTTSETVRAVLDSLSEKKKSSPAAD

SEQ ID NO: 164 - protein, 167 aa, Artificial Sequence
  Description of Artificial Sequence Synthetic IL2RG3.6 variant Amel2 sequence
MANTKYNKEFLLYLAGFVDGDGSIVAQIKPNQGHKFKHQLSLTFTVTQKTQRRWFLDKLVDEIGVGYVRDSGSVSEYRLSKIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 165 - protein, 167 aa, Artificial Sequence
  Description of Artificial Sequence Synthetic IL2RG3.6 variant Amel3 sequence
MANTKYNKEFLLYLAGFVDGDGSIVAQIKPRQSVKFKHQLSLTFTVTQKTRRRWFLDKLVDEIGVGYVRDSGSVSEYRLSKIKPLHNFLTQLQPFLKLKQKQANLVLKIIEQLPSAKESPDKFLEVCTWADQIAALNDSKTRKTTSETVRAVLDSLSEKKKSSPAAD

SEQ ID NO: 166 - protein, 9 aa, Homo sapiens
LAGLIDADG

SEQ ID NO: 167 - protein, 167 aa, Artificial Sequence; identical to SEQ ID NO: 38
  Description of Artificial Sequence Synthetic KNSTQQ/NYSYQ variant sequence

SEQ ID NO: 168 - protein, 167 aa, Artificial Sequence; identical to SEQ ID NO: 43
  Description of Artificial Sequence Synthetic SNSYRK/DNSNI variant sequence

SEQ ID NO: 169 - protein, 167 aa, Artificial Sequence; identical to SEQ ID NO: 120
  Description of Artificial Sequence Synthetic KHTCRS/QRDNR variant sequence

SEQ ID NO: 170 - protein, 167 aa, Artificial Sequence; identical to SEQ ID NO: 121
  Description of Artificial Sequence Synthetic KNDYYS/QRSHY variant sequence

SEQ ID NO: 171 - protein, 167 aa, Artificial Sequence; identical to SEQ ID NO: 122
  Description of Artificial Sequence Synthetic KRANQE/YRSQI variant sequence

SEQ ID NO: 172 - protein, 167 aa, Artificial Sequence; identical to SEQ ID NO: 123
  Description of Artificial Sequence Synthetic KNSCAS/NRSYN variant sequence

SEQ ID NO: 173 - protein, 167 aa, Artificial Sequence; identical to SEQ ID NO: 39
  Description of Artificial Sequence Synthetic KNSTQQ/RYSEY variant sequence

SEQ ID NO: 174 - protein, 167 aa, Artificial Sequence; identical to SEQ ID NO: 44
  Description of Artificial Sequence Synthetic KNTYQS/DYSSR variant sequence

SEQ ID NO: 175 - protein, 167 aa, Artificial Sequence
  Description of Artificial Sequence Synthetic KNSSRE/LRNNI variant sequence
MANTKYNKEFLLYLAGFVDGDGSIIAQIKPNQSSKFKHRLELTFLVTQKTQRRWFLDKLVDEIGVGYVRDNGSVSNYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 176 - protein, 167 aa, Artificial Sequence; identical to SEQ ID NO: 125
  Description of Artificial Sequence Synthetic KDSRTS/AYSYK variant sequence

SEQ ID NO: 177 - protein, 167 aa, Artificial Sequence; identical to SEQ ID NO: 41
  Description of Artificial Sequence Synthetic KNSRNQ/YRSDV variant sequence

SEQ ID NO: 178 - protein, 167 aa, Artificial Sequence; identical to SEQ ID NO: 46
  Description of Artificial Sequence Synthetic KNSTAS/QYSRQ variant sequence

SEQ ID NO: 179 - protein, 167 aa, Artificial Sequence
  Description of Artificial Sequence Synthetic KSSCQA/AYSYI variant sequence
MANTKYNKEFLLYLAGFVDGDGSIIAQIKPSQSCKFKHQLALTFAVTQKTQRRWFLDKLVDEIGVGYVYDSGSVSYYILSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 180 - protein, 167 aa, Artificial Sequence; identical to SEQ ID NO: 127
  Description of Artificial Sequence Synthetic KNTYWS/AYSYK variant sequence

SEQ ID NO: 181 - protein, 167 aa, Artificial Sequence; identical to SEQ ID NO: 42
  Description of Artificial Sequence Synthetic KNRDQS/DNSNI variant sequence

SEQ ID NO: 182 - protein, 167 aa, Artificial Sequence; identical to SEQ ID NO: 47
  Description of Artificial Sequence Synthetic KNSTAS/AYSYK variant sequence

SEQ ID NO: 183 - protein, 167 aa, Artificial Sequence; identical to SEQ ID NO: 64
  Description of Artificial Sequence Synthetic KNSRQY/RYSDT variant sequence

SEQ ID NO: 184 - protein, 167 aa, Artificial Sequence; identical to SEQ ID NO: 53
  Description of Artificial Sequence Synthetic KNSHQS/KYSEV variant sequence

SEQ ID NO: 185 - protein, 167 aa, Artificial Sequence
  Description of Artificial Sequence Synthetic KNSRQS/RYSDT variant sequence
MANTKYNKEFLLYLAGFVDGDGSIIAQIKPNQSRKFKHQLSLTFRVTQKTQRRWFLDKLVDEIGVGYVYDSGSVSDYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 186 - protein, 167 aa, Artificial Sequence
  Description of Artificial Sequence Synthetic KNSHQY/RYSDT variant sequence
MANTKYNKEFLLYLAGFVDGDGSIIAQIKPNQSHKFKHQLYLTFRVTQKTQRRWFLDKLVDEIGVGYVYDSGSVSDYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 187 - protein, 167 aa, Artificial Sequence
  Description of Artificial Sequence Synthetic KNSHQY/KYSEV variant sequence
MANTKYNKEFLLYLAGFVDGDGSIIAQIKPNQSHKFKHQLYLTFKVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 188 - protein, 167 aa, Artificial Sequence; identical to SEQ ID NO: 51
  Description of Artificial Sequence Synthetic KNSRQY/RYSEV variant sequence

SEQ ID NO: 189 - protein, 167 aa, Artificial Sequence
  Description of Artificial Sequence Synthetic KNSHQY/RYSEV variant sequence
MANTKYNKEFLLYLAGFVDGDGSIIAQIKPNQSHKFKHQLYLTFRVTQKTQRRWFLDKLVDEIGVGYVYDSGSVSEYVLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 190 - protein, 167 aa, Artificial Sequence
  Description of Artificial Sequence Synthetic KRSVQS/TRSER variant sequence
MANTKYNKEFLLYLAGFVDGDGSIIAQIKPRQSVKFKHQLSLTFTVTQKTQRRWFLDKLVDEIGVGYVRDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 191 - protein, 167 aa, Artificial Sequence
  Description of Artificial Sequence Synthetic KRSSQS/RYSET variant sequence
MANTKYNKEFLLYLAGFVDGDGSIIAQIKPRQSSKFKHQLSLTFRVTQKTQRRWFLDKLVDEIGVGYVYDSGSVSEYTLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 192 - protein, 167 aa, Artificial Sequence
  Description of Artificial Sequence Synthetic KNGHQS/TRSER variant sequence
MANTKYNKEFLLYLAGFVDGDGSIIAQIKPNQGHKFKHQLSLTFTVTQKTQRRWFLDKLVDEIGVGYVRDSGSVSEYRLSEIKPLHNFLTQLQPFLKLKQKQANLVLKIIEQLPSAKESPDKFLEVCTWVDQIAALNDSKTRKTTSETVRAVLDSLSEKKKSSPAAD

SEQ ID NO: 193 - protein, 167 aa, Artificial Sequence
  Description of Artificial Sequence Synthetic sequence
MAXTXYNXXFLLYLAGFVDXDGSIXAXIKPNXSYXFKHQXSLTXQVTQKTXRXWXLDXLXXEIGXGYVRDRXSVSNYILXXIXPLHNXLXQLQPFLXLXQXQAXLXLXIIEXLPSAKXSPDXXLEVCXWXDQXAAXNDSXXRKXTSEXVRAVLXXLXXKXXSXXAAD

SEQ ID NO: 194 - protein, 167 aa, Artificial Sequence
  Description of Artificial Sequence Synthetic sequence
MANTKYNKEFLLYLAGFVDXDGSIXAQIKPNQSYKFKHQLSLTFQVTQKTQRRWXLDKLVDEIGVGYVRDRGSVSNYILSXIKPLHNXLTQLQPFLKLKQKQANLXLKIIEQLPSAKESPDKFLEVCTWVDQXAALNDSKTRKTTSETVRAVLDSLSEKKKSSPAAD

SEQ ID NO: 195 - protein, 167 aa, Artificial Sequence; identical to SEQ ID NO: 102
  Description of Artificial Sequence Synthetic I-CreI variant .4_R7 sequence

(19) United States
(12) Patent Application Publication
(10) Pub. No.: US 2014/0017731 A1
(43) Pub. Date: Jan. 16, 2014

(54) MEGANUCLEASE VARIANTS CLEAVING A DNA TARGET SEQUENCE FROM THE HUMAN INTERLEUKIN-2 RECEPTOR GAMMA CHAIN GENE AND USES THEREOF

(71) Applicant: CELLECTIS, Paris (FR)

(72) Inventors: Agnes GOUBLE, Paris (FR); Sylvestre Grizot, La Garenne Colombes (FR)

(73) Assignee: CELLECTIS, Paris (FR)

(21) Appl. No.: 13/904,793

(22) Filed: May 29, 2013

Related U.S. Application Data
(62) Division of application No. 12/671,853, filed on Apr. 15, 2010, now abandoned.

(30) Foreign Application Priority Data
Aug. 3, 2007 (IB) PCT/IB2007/003232

(51) Int. Cl. C12P 19/34

(52) U.S. Cl. CPC C12P 19/34
USPC 435/91.53

(57) ABSTRACT

An I-CreI variant, wherein at least one of the two I-CreI monomers has at least two substitutions, one in each of the two functional subdomains of the LAGLIDADG core domain situated respectively from positions 26 to 40 and 44 to 77 of I-CreI, said variant being able to cleave a DNA target sequence from the human IL2RG gene. Use of said variant and derived products for the prevention and the treatment of X-linked severe combined immunodeficiency.

### CROSS-REFERENCE TO RELATED APPLICATIONS

The present application is a division of U.S. Ser. No. 12/671,853, filed Apr. 15, 2010, which is a 35 U.S.C. §371 National Stage patent application of International patent application PCT/IB2008/002999, filed on Aug. 4, 2008, which claims priority to International patent application PCT/IB2007/003232, filed Aug. 3, 2007.

The invention relates to a meganuclease variant cleaving a DNA target sequence from the human interleukin-2 receptor gamma chain (IL2RG) gene, also named common cytokine receptor gamma chain gene or gamma C (γC) gene, to a vector encoding said variant, to a cell, an animal or a plant modified by said vector and to the use of said meganuclease variant and derived products for genome therapy ex vivo (gene cell therapy), and genome engineering.

Severe Combined Immune Deficiency (SCID) results from a defect in lymphocytes T maturation, always associated with a functional defect in lymphocytes B (Cavazzana-Calvo et al., Annu. Rev. Med., 2005, 56, 585-602; Fischer et al., Immunol. Rev., 2005, 203, 98-109). Overall incidence is estimated to 1 in 75 000 births. Patients with untreated SCID are subject to multiple opportunist microorganism infections, and do generally not live beyond one year. SCID can be treated by allogenic hematopoietic stem cell transfer, from a familial donor. Histocompatibility with the donor can vary widely. In the case of Adenosine Deaminase (ADA) deficiency, one of the SCID forms, patients can be treated by injection of recombinant Adenosine Deaminase enzyme.

Since the ADA gene has been shown to be mutated in SCID patients (Giblett et al., Lancet, 1972, 2, 1067-1069), several other genes involved in SCID have been identified (Cavazzana-Calvo et al., Annu. Rev. Med., 2005, 56, 585-602; Fischer et al., Immunol. Rev., 2005, 203, 98-109). There are four major causes for SCID: (i) the most frequent form of SCID, SCID-X1 (X-linked SCID or X-SCID), is caused by mutation in the IL2RG gene, resulting in the absence of mature T lymphocytes and NK cells. IL2RG encodes the γC protein (Noguchi, et al., Cell, 1993, 73, 147-157), a common component of at least five interleukin receptor complexes. These receptors activate several targets through the JAK3 kinase (Macchi et al., Nature, 1995, 377, 65-68), which inactivation results in the same syndrome as γC inactivation; (ii) mutation in the ADA gene results in a defect in purine metabolism that is lethal for lymphocyte precursors, which in turn results in the quasi absence of B, T and NK cells; (iii) V(D)J recombination is an essential step in the maturation of immunoglobulins and T lymphocytes receptors (TCRs). Mutations in Recombination Activating Gene 1 and 2 (RAG1 and RAG2) and Artemis, three genes involved in this process, result in the absence of mature T and B lymphocytes; and (iv) Mutations in other genes such as CD45, involved in T cell specific signaling have also been reported, although they represent a minority of cases (Cavazzana-Calvo et al., Annu. Rev. Med., 2005, 56, 585-602; Fischer et al., Immunol. Rev., 2005, 203, 98-109).

Since when their genetic bases have been identified, the different SCID forms have become a paradigm for gene therapy approaches (Fischer et al., Immunol. Rev., 2005, 203, 98-109) for two major reasons.

First, as in all blood diseases, an ex vivo treatment can be envisioned. Hematopoietic Stem Cells (HSCs) can be recovered from bone marrow, and keep their pluripotent properties for a few cell divisions. Therefore, they can be treated in vitro, and then reinjected into the patient, where they repopulate the bone marrow.

Second, since the maturation of lymphocytes is impaired in SCID patients, corrected cells have a selective advantage. Therefore, a small number of corrected cells can restore a functional immune system. This hypothesis was validated several times by (i) the partial restoration of immune functions associated with the reversion of mutations in SCID patients (Hirschhorn et al., Nat. Genet., 1996, 13, 290-295; Stephan et al., N. Engl. J. Med., 1996, 335, 1563-1567; Bousso et al., Proc. Natl., Acad. Sci. USA, 2000, 97, 274-278; Wada et al., Proc. Natl. Acad. Sci. USA, 2001, 98, 8697-8702; Nishikomori et al., Blood, 2004, 103, 4565-4572), (ii) the correction of SCID-X1 deficiencies in vitro in hematopoietic cells (Candotti et al., Blood, 1996, 87, 3097-3102; Cavazzana-Calvo et al., Blood, 1996, Blood, 88, 3901-3909; Taylor et al., Blood, 1996, 87, 3103-3107; Hacein-Bey et al., Blood, 1998, 92, 4090-4097), (iii) the correction of SCID-X1 (Soudais et al., Blood, 2000, 95, 3071-3077; Tsai et al., Blood, 2002, 100, 72-79), JAK-3 (Bunting et al., Nat. Med., 1998, 4, 58-64; Bunting et al., Hum. Gene Ther., 2000, 11, 2353-2364) and RAG2 (Yates et al., Blood, 2002, 100, 3942-3949) deficiencies in vivo in animal models and (iv) by the result of gene therapy clinical trials (Cavazzana-Calvo et al., Science, 2000, 288, 669-672; Aiuti et al., Nat. Med., 2002, 8, 423-425; Gaspar et al., Lancet, 2004, 364, 2181-2187).

Since the nineties, several gene therapy clinical trials have generated a large body of very useful information. These studies are all based on the complementation of the mutated gene with a functional gene introduced into the genome with a viral vector. Clinical trial for SCID-X1 (γC deficiency) resulted in the restoration of a functional or partly functional immune system in nine out of ten patients treated by gene therapy (Cavazzana-Calvo et al., Science, 2000, 288, 669-672). Other successful clinical trials were conducted with four SCID-X1 patients (Gaspar et al., Lancet, 2004, 364, 2181-2187) and four ADA patients (Aiuti et al., Science, 2002, 296, 2410-2413), confirming the benefits of the gene therapy approach. However, the first trials have also illustrated the risks associated with this approach. Later, three patients developed a monoclonal lymphoproliferation, closely mimicking acute leukemia. These lymphoproliferations are associated with the activation of cellular oncogenes by insertional mutagenesis. In all three cases, proliferating cells are characterized by the insertion of the retroviral vector in the same locus, resulting in overexpression of the LMO2 gene (Hacein-Bey et al., Science, 2003, 302, 415-419; Fischer et al., N. Engl. J. Med., 2004, 350, 2526-2527).

Thus, these results have demonstrated both the extraordinary potential of a <<genomic therapy>> in the treatment of inherited diseases, and the limits of the integrative retroviral vectors (Kohn et al., Nat. Rev. Cancer, 2003, 3, 477-488). Despite the development of novel electroporation methods (Nucleofector® technology from AMAXA GmbH; PCT/EP01/07348, PCT/DE02/01489 and PCT/DE02/01483), viral vectors have so far given the most promising results in HSCs. Retrovirus derived from the MoMLV (Moloney Murine Leukemia Virus) have been used to transduce HSCs efficiently, including for clinical trials (see above). However, classical retroviral vectors transduce only cycling cells, and transduction of HSCs with Moloney vectors requires their stimulation and the induction of mitosis with growth factors, thus strongly compromising their pluripotent properties ex vivo. In contrast, lentiviral vectors derived from HIV-1, can efficiently transduce non mitotic cells, and are perfectly adapted to HSCs transduction (Logan et al., Curr. Opin. Biotechnol., 2002, 13, 429-436). With such vectors, the insertion of flap DNA strongly stimulates entry into the nucleus, and thereby the rate of HSC transduction (Sirven et al., Blood, 2000, 96, 4103-4110; Zennou et al., Cell, 2000, 101, 173-185). However, lentivirial vectors are also integrative, with same potential risks as Moloney vectors: following insertion into the genome, the virus LTRs promoters and enhancers can stimulate the expression of adjacent genes (see above). Deletion of enhancer and promoter of the U3 region from LTR3′ can be an option. After retrotranscription, this deletion will be duplicated into the LTR5′, and these vectors, called <<delta U3>> or <<Self Inactivating>>, can circumvent the risks of insertional mutagenesis resulting from the activation of adjacent genes. However, they do not abolish the risks of gene inactivation by insertion, or of transcription readthrough.

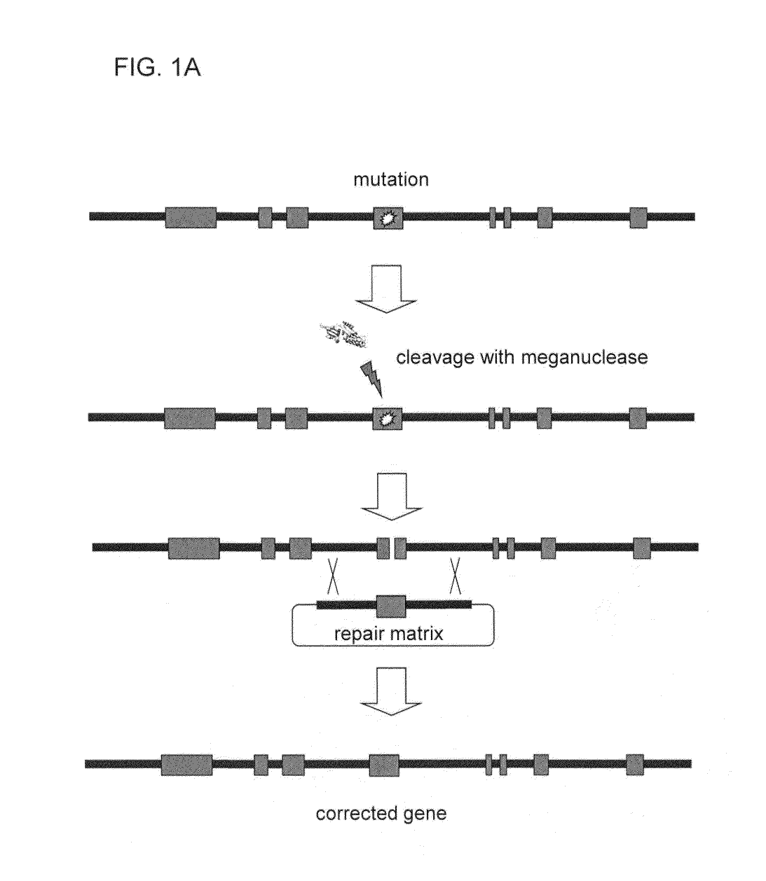

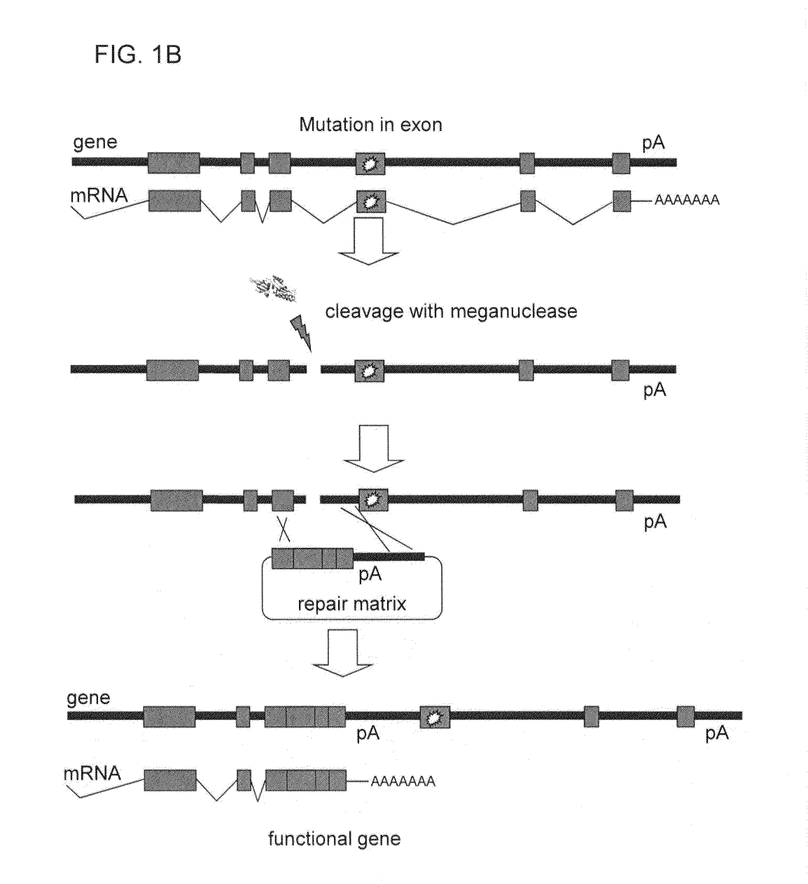

Targeted homologous recombination is another alternative that should bypass the problems raised by current approaches. Current gene therapy strategies are based on a complementation approach, wherein randomly inserted but functional extra copy of the gene provide for the function of the mutated endogenous copy. In contrast, homologous recombination should allow for the precise correction of mutations in situ (FIG. 1A). Homologous recombination (HR), is a very conserved DNA maintenance pathway involved in the repair of DNA double-strand breaks (DSBs) and other DNA lesions (Rothstein, Methods Enzymol., 1983, 101, 202-211; Paques et al., Microbiol Mol Biol Rev, 1999, 63, 349-404; Sung et al., Nat. Rev. Mol. Cell. Biol., 2006, 7, 739-750) but it also underlies many biological phenomenon, such as the meiotic reassortiment of alleles in meiosis (Roeder, Genes Dev., 1997, 11, 2600-2621), mating type interconversion in yeast (Haber, Annu. Rev. Genet., 1998, 32, 561-599), and the “homing” of class I introns and inteins to novel alleles. HR usually promotes the exchange of genetic information between endogenous sequences, but in gene targeting experiments, it is used to promote exchange between an endogenous chromosomal sequence and an exogenous DNA construct. Basically, a DNA sharing homology with the targeted sequence was introduced into the cell's nucleus, and the endogenous homologous recombination machinery provides for the next steps (FIG. 1A).

Homologous gene targeting strategies have been used to knock out endogenous genes (Capecchi, M. R., Science, 1989, 244, 1288-1292, Smithies, O., Nature Medicine, 2001, 7, 1083-1086) or knock-in exogenous sequences in the chromosome. It can as well be used for gene correction, and in principle, for the correction of mutations linked with monogenic diseases. However, this application is in fact difficult, due to the low efficiency of the process (10⁻⁶ to 10⁻⁹ of transfected cells).

In the last decade, several methods have been developed to enhance this yield. For example, chimeraplasty (De Semir et al. J. Gene Med., 2003, 5, 625-639) and Small Fragment Homologous Replacement (Goncz et al., Gene Ther, 2001, 8, 961-965; Bruscia et al., Gene Ther., 2002, 9, 683-685; Sangiuolo et al., BMC Med. Genet., 2002, 3, 8; De Semir, D. and J. M. Aran, Oligonucleotides, 2003, 13, 261-269) have both been used to try to correct CFTR mutations with various levels of success.

Another strategy to enhance the efficiency of recombination is to deliver a DNA double-strand break in the targeted locus, using meganucleases. Meganucleases are by definition sequence-specific endonucleases recognizing large sequences (Thierry, A. and B. Dujon, Nucleic Acids Res., 1992, 20, 5625-5631). They can cleave unique sites in living cells, thereby enhancing gene targeting by 1000-fold or more in the vicinity of the cleavage site (Puchta et al., Nucleic Acids Res., 1993, 21, 5034-5040 ; Rouet et al., Mol. Cell. Biol., 1994, 14, 8096-8106 ; Choulika et al., Mol. Cell. Biol., 1995, 15, 1968-1973; Puchta et al., Proc. Natl. Acad. Sci. U.S.A., 1996, 93, 5055-5060 ; Sargent et al., Mol. Cell. Biol., 1997, 17, 267-277; Cohen-Tannoudji et al., Mol. Cell. Biol., 1998, 18, 1444-1448 ; Donoho, et al., Mol. Cell. Biol., 1998, 18, 4070-4078; Elliott et al., Mol. Cell. Biol., 1998, 18, 93-101). Such meganucleases could be used to correct mutations responsible for monogenic inherited diseases.

The most accurate way to correct a genetic defect is to use a repair matrix with a non mutated copy of the gene, resulting in a reversion of the mutation. However, the efficiency of gene correction decreases as the distance between the mutation and the DSB grows, with a five-fold decrease by 200 by of distance. Therefore, a given meganuclease can be used to correct only mutations in the vicinity of its DNA target (FIG. 1A).

An alternative, termed “exon knock-in” is featured in FIG. 1B. In this case, a meganuclease cleaving in the 5′ part of the gene can be used to knock-in functional exonic sequences upstream of the deleterious mutation. Although this method places the transgene in its regular location, it also results in exons duplication, which impact on the long range remains to be evaluated. In addition, should naturally cis-acting elements be placed in an intron downstream of the cleavage, their immediate environment would be modified and their proper function would also need to be explored. However, this method has a tremendous advantage: a single meganuclease could be used for many different mutations downstream of the meganuclease cleavage site.

However, although several hundreds of natural meganucleases, also referred to as “homing endonucleases” have been identified (Chevalier, B. S. and B. L. Stoddard, Nucleic Acids Res., 2001, 29, 3757-3774), the repertoire of cleavable sequences is too limited to address the complexity of the genomes, and there is usually no cleavable site in a chosen gene. For example, there is no cleavage site for a known natural meganuclease in human SCID genes. Theoretically, the making of artificial sequence specific endonucleases with chosen specificities could alleviate this limit. Therefore, the making of meganucleases with tailored specificities is under intense investigation.

Recently, fusion of Zinc-Finger Proteins (ZFPs) with the catalytic domain of the FokI, a class IIS restriction endonuclease, were used to make functional sequence-specific endonucleases (Smith et al., Nucleic Acids Res., 1999, 27, 674-681; Bibikova et al., Mol. Cell. Biol., 2001, 21, 289-297; Bibikova et al., Genetics, 2002, 161, 1169-1175; Bibikova et al., Science, 2003, 300, 764; Porteus, M. H. and D. Baltimore, Science, 2003, 300, 763-; Alwin et al., Mol. Ther., 2005, 12, 610-617; Urnov et al., Nature, 2005, 435, 646-651; Porteus, M. H., Mol. Ther., 2006, 13, 438-446). Such nucleases could recently be used for the engineering of the ILR2G gene in human cells from the lymphoid lineage (Urnov et al., Nature, 2005, 435, 646-651).

The binding specificity of Cys2-His2 type Zinc-Finger Proteins, is easy to manipulate, probably because they represent a simple (specificity driven by essentially four residues per finger), and modular system (Pabo et al., Annu. Rev. Biochem., 2001, 70, 313-340; Jamieson et al., Nat. Rev. Drug Discov., 2003, 2, 361-368). Studies from the Pabo laboratories resulted in a large repertoire of novel artificial ZFPs, able to bind most G/ANNG/ANNG/ANN sequences (Rebar, E. J. and C. O. Pabo, Science, 1994, 263, 671-673; Kim, J. S. and C. O. Pabo, Proc. Natl. Acad. Sci. USA, 1998, 95, 2812-2817, Klug Choo, Y. and A. Klug, Proc. Natl. Acad. Sci. USA, 1994, 91, 11163-11167; Isalan M. and A. Klug, Nat. Biotechnol., 2001, 19, 656-660 and Barbas Choo, Y. and A. Klug, Proc. Natl. Acad. Sci. USA, 1994, 91, 11163-11167; Isalan M. and A. Klug, Nat. Biotechnol., 2001, 19, 656-660).

Nevertheless, ZFPs might have their limitations, especially for applications requiring a very high level of specificity, such as therapeutic applications. The FokI nuclease activity in fusion acts as a dimer, but it was recently shown that it could cleave DNA when only one out of the two monomers was bound to DNA, or when the two monomers were bound to two distant DNA sequences (Catto et al., Nucleic Acids Res., 2006, 34, 1711-1720). Thus, specificity might be very degenerate, as illustrated by toxicity in mammalian cells (Porteus, M. H. and D. Baltimore, Science, 2003, 300, 763) and Drosophila (Bibikova et al., Genetics, 2002, 161, 1169-1175; Bibikova et al., Science, 2003, 300, 764-.).

In the wild, meganucleases are essentially represented by homing endonucleases. Homing Endonucleases (HEs) are a widespread family of natural meganucleases including hundreds of proteins families (Chevalier, B. S. and B. L. Stoddard, Nucleic Acids Res., 2001, 29, 3757-3774). These proteins are encoded by mobile genetic elements which propagate by a process called “homing”: the endonuclease cleaves a cognate allele from which the mobile element is absent, thereby stimulating a homologous recombination event that duplicates the mobile DNA into the recipient locus. Given their exceptional cleavage properties in terms of efficacy and specificity, they could represent ideal scaffold to derive novel, highly specific endonucleases.

HEs belong to four major families. The LAGLIDADG (SEQ ID NO: 166) family, named after a conserved peptidic motif involved in the catalytic center, is the most widespread and the best characterized group. Seven structures are now available. Whereas most proteins from this family are monomeric and display two LAGLIDADG motifs (SEQ ID NO: 166), a few ones have only one motif, but dimerize to cleave palindromic or pseudo-palindromic target sequences.

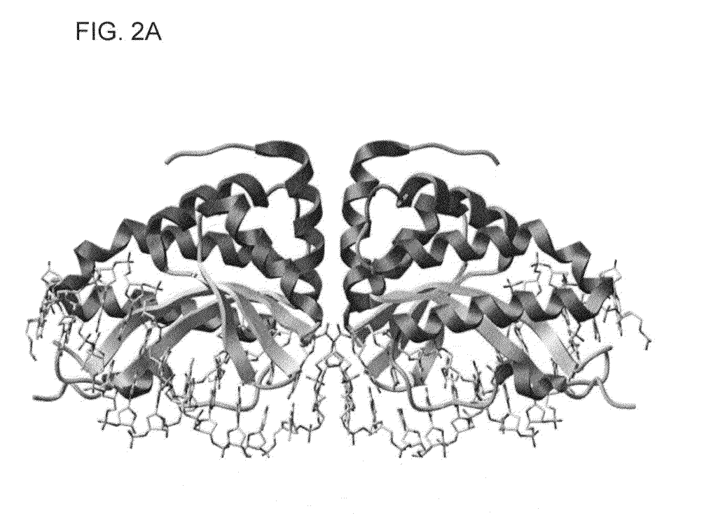

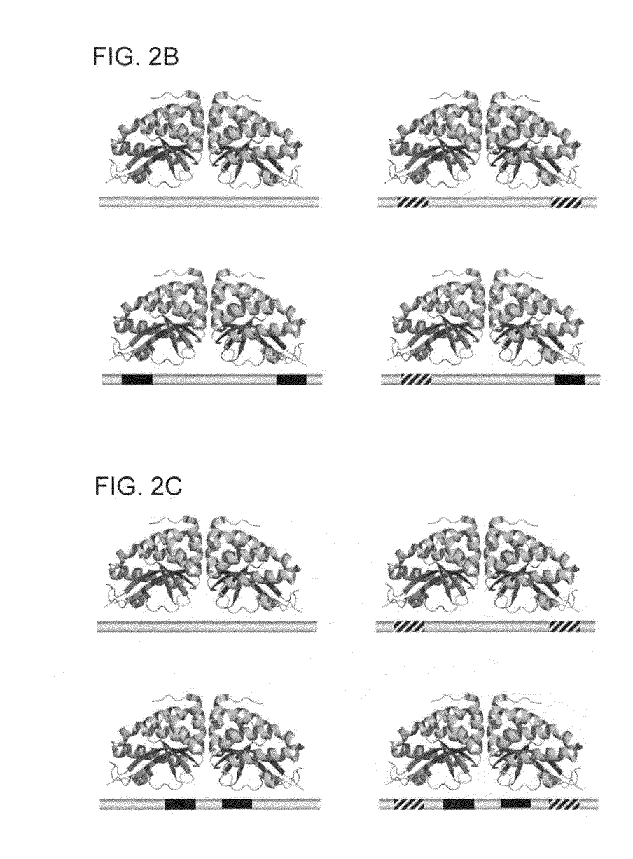

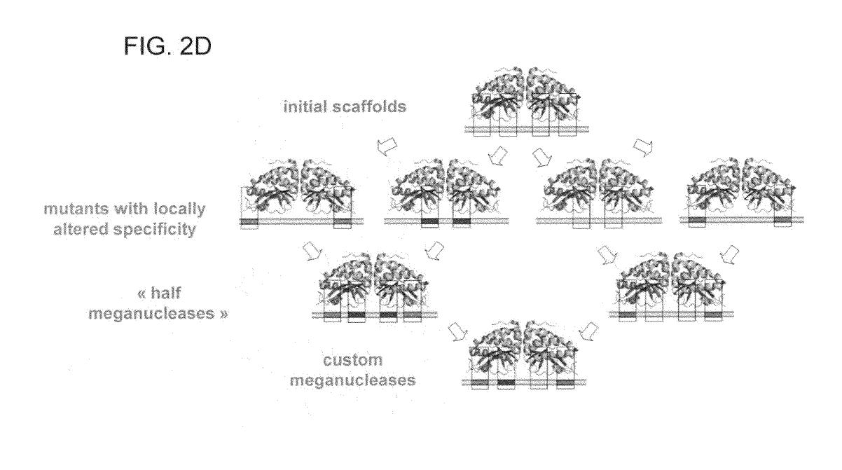

Although the LAGLIDADG peptide (SEQ ID NO: 166) is the only conserved region among members of the family, these proteins share a very similar architecture (FIG. 2A). The catalytic core is flanked by two DNA-binding domains with a perfect two-fold symmetry for homodimers such as I-CreI (Chevalier, et al., Nat. Struct. Biol., 2001, 8, 312-316), I-MsoI (Chevalier et al., J. Mol. Biol., 2003, 329, 253-269) and I-CeuI (Spiegel et al., Structure, 2006, 14, 869-880) and with a pseudo symmetry for monomers such as I-SceI (Moure et al., J. Mol. Biol., 2003, 334, 685-69), I-DmoI (Silva et al., J. Mol. Biol., 1999, 286, 1123-1136) or I-AniI (Bolduc et al., Genes Dev., 2003, 17, 2875-2888). Both monomers, or both domains (for monomeric proteins) contribute to the catalytic core, organized around divalent cations. Just above the catalytic core, the two LAGLIDADG peptides (SEQ ID NO: 166) play also an essential role in the dimerization interface. DNA binding depends on two typical saddle-shaped αββαββα folds, sitting on the DNA major groove. Other domains can be found, for example in inteins such as PI-PfuI (Ichiyanagi et al., J. Mol. Biol., 2000, 300, 889-901) and PI-SceI (Moure et al., Nat. Struct. Biol., 2002, 9, 764-770), which protein splicing domain is also involved in DNA binding.

The making of functional chimeric meganucleases, by fusing the N-terminal I-DmoI domain with an I-CreI monomer (Chevalier et al., Mol. Cell., 2002, 10, 895-905; Epinat et al., Nucleic Acids Res, 2003, 31, 2952-62; International PCT Applications WO 03/078619 and WO 2004/031346) have demonstrasted the plasticity of LAGLIDADG proteins (SEQ ID NO: 166).

Besides, different groups have used a semi-rational approach to locally alter the specificity of I-CreI (Seligman et al., Genetics, 1997, 147, 1653-1664; Sussman et al., J. Mol. Biol., 2004, 342, 31-41; International PCT Applications WO 2006/097784, WO 2006/097853, WO 2007/060495 and WO 2007/049156; Arnould et al., J. Mol. Biol., 2006, 355, 443-458; Rosen et al., Nucleic Acids Res., 2006, 34, 4791-4800 ; Smith et al., Nucleic Acids Res., 2006, 34, e149), I-SceI (Doyon et al., J. Am. Chem. Soc., 2006, 128, 2477-2484), PI-SceI (Gimble et al., J. Mol. Biol., 2003, 334, 993-1008) and I-MsoI (Ashworth et al., Nature, 2006, 441, 656-659).

In addition, hundreds of I-CreI derivatives with locally altered specificity were engineered by combining the semi-rational approach and High Throughput Screening:

Residues Q44, R68 and R70 or Q44, R68, D75 and 177 of I-CreI were mutagenized and a collection of variants with altered specificity at positions ±3 to 5 of the DNA target (5NNN DNA target) were identified by screening (International PCT Applications WO 2006/097784 and WO 2006/097853; Arnould et al., J. Mol. Biol., 2006, 355, 443-458; Smith et al., Nucleic Acids Res., 2006, 34, e149).

Residues K28, N30 and Q38, N30, Y33 and Q38 or K28, Y33, Q38 and S40 of I-CreI were mutagenized and a collection of variants with altered specificity at positions ±8 to 10 of the DNA target (10NNN DNA target) were identified by screening (Smith et al., Nucleic Acids Res., 2006, 34, e149; International PCT Applications WO 2007/060495 and WO 2007/049156).

Two different variants were combined and assembled in a functional heterodimeric endonuclease able to cleave a chimeric target resulting from the fusion of a different half of each variant DNA target sequence (Arnould et al., precited; International PCT Applications WO 2006/097854 and WO 2007/034262), as illustrated on FIG. 2B.

Furthermore, residues 28 to 40 and 44 to 77 of I-CreI were shown to form two separable functional subdomains, able to bind distinct parts of a homing endonuclease half-site (Smith et al. Nucleic Acids Res., 2006, 34, e149; International PCT Applications WO 2007/049095 and WO 2007/057781).

The combination of mutations from the two subdomains of I-CreI within the same monomer allowed the design of novel chimeric molecules (homodimers) able to cleave a palindromic combined DNA target sequence comprising the nucleotides at positions ±3 to 5 and ±8 to 10 which are bound by each subdomain (Smith et al., Nucleic Acids Res., 2006, 34, e149; International PCT Applications WO 2007/049095 and WO 2007/057781), as illustrated on FIG. 2C.

The combination of the two former steps allows a larger combinatorial approach, involving four different subdomains. The different subdomains can be modified separately and combined to obtain an entirely redesigned meganuclease variant (heterodimer or single-chain molecule) with chosen specificity, as illustrated on FIG. 2D. In a first step, couples of novel meganucleases are combined in new molecules (“half-meganucleases”) cleaving palindromic targets derived from the target one wants to cleave. Then, the combination of such “half-meganuclease” can result in a heterodimeric species cleaving the target of interest. The assembly of four set of mutations into heterodimeric endonucleases cleaving a model target sequence or a sequence from the human RAG1 and XPC genes have been described in Smith et al. (Nucleic Acids Res., 2006, 34, e149) and Arnould et al., (J. Mol. Biol., 2007, 371, 49-65), respectively.

These variants can be used to cleave genuine chromosomal sequences and have paved the way for novel perspectives in several fields, including gene therapy.

However, even though the base-pairs ±1 and ±2 do not display any contact with the protein, it has been shown that these positions are not devoid of content information (Chevalier et al., J. Mol. Biol., 2003, 329, 253-269), especially for the base-pair ±1 and could be a source of additional substrate specificity (Argast et al., J. Mol. Biol., 1998, 280, 345-353; Jurica et al., Mol. Cell., 1998, 2, 469-476; Chevalier, B. S. and B. L. Stoddard, Nucleic Acids Res., 2001, 29, 3757-3774). In vitro selection of cleavable I-CreI target (Argast et al., precited) randomly mutagenized, revealed the importance of these four base-pairs on protein binding and cleavage activity. It has been suggested that the network of ordered water molecules found in the active site was important for positioning the DNA target (Chevalier et al., Biochemistry, 2004, 43, 14015-14026). In addition, the extensive conformational changes that appear in this region upon I-CreI binding suggest that the four central nucleotides could contribute to the substrate specificity, possibly by sequence dependent conformational preferences (Chevalier et al., 2003, precited).

Thus, it was not clear if mutants identified on 10NNN and 5NNN DNA targets as homodimers cleaving a palindromic sequence with the four central nucleotides being gtac, would allow the design of new endonucleases that would cleave targets containing changes in the four central nucleotides.

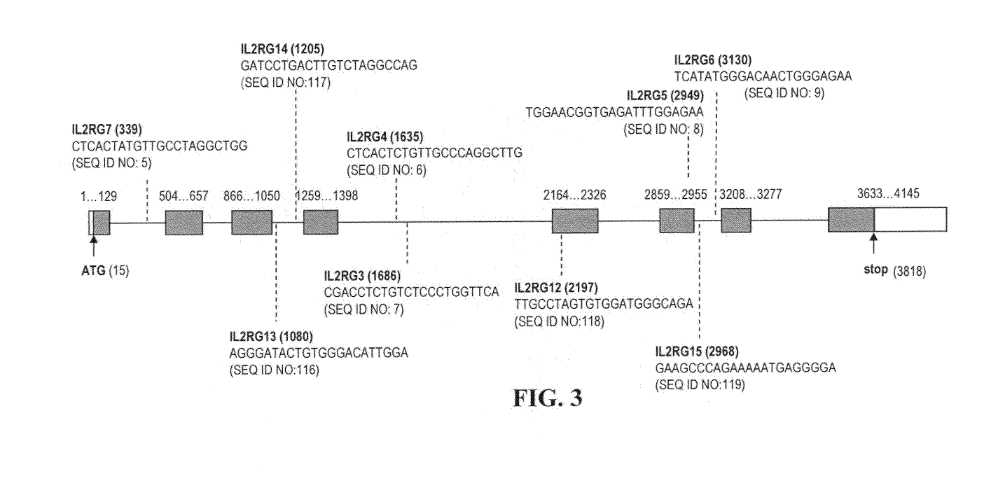

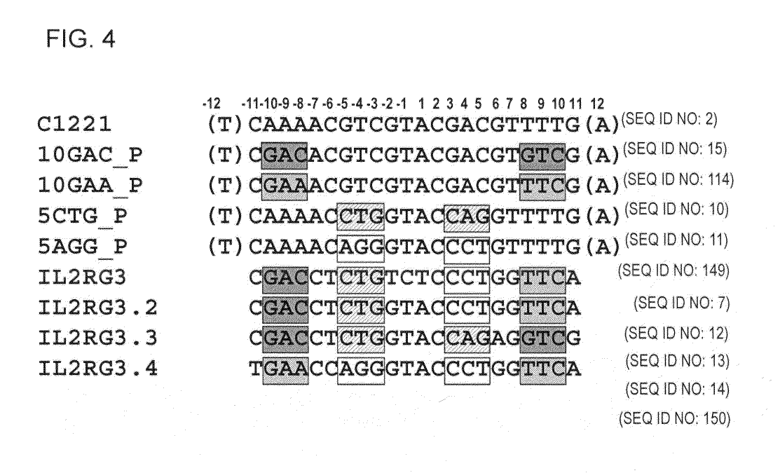

The Inventors have identified a series of DNA targets in the human IL2RG gene that could be cleaved by I-CreI variants (Table I and FIG. 3). The combinatorial approach described in FIG. 2D was used to entirely redesign the DNA binding domain of the I-CreI protein and thereby engineer novel meganucleases with fully engineered specificity, to cleave one DNA target (IL2RG3) from the human IL2RG gene, which differs from the I-CreI C1221 22 by palindromic site by 15 nucleotides including three (positions −2, −1, +1) out of the four central nucleotides (FIG. 4).

Even though the combined variants were initially identified towards nucleotides 10NNN and 5NNN respectively, and a strong impact of the four central nucleotides of the target on the activity of the engineered meganuclease was observed, functional meganucleases with a profound change in specificity were selected. Furthermore, the activity of the engineered protein could be significantly improved by random and/or site-directed mutagenesis and screening, to compare with the activity of the I-CreI protein.

The I-CreI variants which are able to cleave a genomic DNA target from the human IL2RG gene can be used for genome therapy of X-linked Severe Combined Immunodeficiency (SCID-X1) and genome engineering at the IL2RG locus.

For example, the DNA target named IL2RG3 is located in intron 4 of the human IL2RG gene (FIG. 3). Gene correction could be used to correct mutations in the vicinity of the cleavage site (FIG. 1A). Since the efficiency of gene correction decreases when the distance to the DSB increases (Elliott et al., Mol. Cell. Biol., 1998, 18, 93-101), this strategy would be most efficient with mutations located within 500 by of the cleavage site. This strategy could be used to correct mutations in exon 4. Alternatively, meganucleases cleaving the IL2RG3 sequence could be used to knockin exonic sequences that would restore a functional IL2RG gene at the IL2RG locus (FIG. 1B). This strategy could be used for any mutation located downstream of the cleavage site.

The invention relates to an I-CreI variant wherein at least one of the two I-CreI monomers has at least two substitutions, one in each of the two functional subdomains of the LAGLIDADG core domain (SEQ ID NO: 166) situated respectively from positions 26 to 40 and 44 to 77 of I-CreI, and is able to cleave a DNA target sequence from the human IL2RG gene.

The cleavage activity of the variant according to the invention may be measured by any well-known, in vitro or in vivo cleavage assay, such as those described in the International PCT Application WO 2004/067736; Epinat et al., Nucleic Acids Res., 2003, 31, 2952-2962; Chames et al., Nucleic Acids Res., 2005, 33, e178; Arnould et al., J. Mol. Biol., 2006, 355, 443-458, and Arnould et al., J. Mol. Biol., 2007, 371, 49-65. For example, the cleavage activity of the variant of the invention may be measured by a direct repeat recombination assay, in yeast or mammalian cells, using a reporter vector. The reporter vector comprises two truncated, non-functional copies of a reporter gene (direct repeats) and the genomic (non-palindromic) DNA target sequence within the intervening sequence, cloned in a yeast or a mammalian expression vector. Usually, the genomic DNA target sequence comprises one different half of each (palindromic or pseudo-palindromic) parent homodimeric I-CreI meganuclease target sequence. Expression of the heterodimeric variant results in a functional endonuclease which is able to cleave the genomic DNA target sequence. This cleavage induces homologous recombination between the direct repeats, resulting in a functional reporter gene (LacZ, for example), whose expression can be monitored by an appropriate assay. The specificity of the cleavage by the variant may be assessed by comparing the cleavage of the (non-palindromic) DNA target sequence with that of the two palindromic sequences cleaved by the parent I-CreI homodimeric meganucleases or compared with wild type I-CreI or I-SceI activity against their natural target.

Definitions

Amino acid residues in a polypeptide sequence are designated herein according to the one-letter code, in which, for example, Q means Gln or Glutamine residue, R means Arg or Arginine residue and D means Asp or Aspartic acid residue.

Nucleotides are designated as follows: one-letter code is used for designating the base of a nucleoside: a is adenine, t is thymine, c is cytosine, and g is guanine For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.

by “meganuclease”, is intended an endonuclease having a double-stranded DNA target sequence of 12 to 45 bp. Said meganuclease is either a dimeric enzyme, wherein each domain is on a monomer or a monomeric enzyme comprising the two domains on a single polypeptide.

by “meganuclease domain” is intended the region which interacts with one half of the DNA target of a meganuclease and is able to associate with the other domain of the same meganuclease which interacts with the other half of the DNA target to form a functional meganuclease able to cleave said DNA target.

by “meganuclease variant” or “variant” is intended a meganuclease obtained by replacement of at least one residue in the amino acid sequence of the wild-type meganuclease (natural meganuclease) with a different amino acid.

by “functional variant” is intended a variant which is able to cleave a DNA target sequence, preferably said target is a new target which is not cleaved by the parent meganuclease. For example, such variants have amino acid variation at positions contacting the DNA target sequence or interacting directly or indirectly with said DNA target.

by “I-CreI” is intended the wild-type I-CreI having the sequence of pdb accession code 1g9y, corresponding to the sequence SEQ ID NO: 1 in the sequence listing.

by “I-CreI variant with novel specificity” is intended a variant having a pattern of cleaved targets different from that of the parent meganuclease. The terms “novel specificity”, “modified specificity”, “novel cleavage specificity”, “novel substrate specificity” which are equivalent and used indifferently, refer to the specificity of the variant towards the nucleotides of the DNA target sequence.

by “I-CreI site” is intended a 22 to 24 by double-stranded DNA sequence which is cleaved by I-CreI. I-CreI sites include the wild-type (natural) non-palindromic I-CreI homing site and the derived palindromic sequences such as the sequence 5′-t₋₁₂c₋₁₁a₋₁₀a₋₉a₋₈a₋₇c₋₆g₋₅t₋₄c₋₃g₋₂t₋₁a₊₁c₊₂g₊₃a_{′4}c₊₅g₊₆t₊₇t₊₈t₊₉t₋₁₀g₊₁₁a₊₁₂ (SEQ ID NO: 2), also called C1221 (FIG. 4).

by “domain” or “core domain” is intended the “LAGLIDADG homing endonuclease core domain” (SEQ ID NO: 166) which is the characteristic α₁β₁β₂α₂β₃β₄α₃ fold of the homing endonucleases of the LAGLIDADG (SEQ ID NO: 166) family, corresponding to a sequence of about one hundred amino acid residues. Said domain comprises four beta-strands (β₁β₂β₃β₄) folded in an antiparallel beta-sheet sheet which interacts with one half of the DNA target. This domain is able to associate with another LAGLIDADG homing endonuclease core domain (SEQ ID NO: 166) which interacts with the other half of the DNA target to form a functional endonuclease able to cleave said DNA target. For example, in the case of the dimeric homing endonuclease I-CreI (163 amino acids), the LAGLIDADG homing endonuclease core domain (SEQ ID NO: 166) corresponds to the residues 6 to 94.

by “subdomain” is intended the region of a LAGLIDADG homing endonuclease core domain (SEQ ID NO: 166) which interacts with a distinct part of a homing endonuclease DNA target half-site.

by “beta-hairpin” is intended two consecutive beta-strands of the antiparallel beta-sheet of a LAGLIDADG homing endonuclease core domain (SEQ ID NO: 166) ((β₁β₂ or ,β₃β₄) which are connected by a loop or a turn,

by “single-chain meganuclease”, “single-chain chimeric meganuclease”, “single-chain meganuclease derivative”, “single-chain chimeric meganuclease derivative” or “single-chain derivative” is intended a meganuclease comprising two LAGLIDADG homing endonuclease domains or core domains (SEQ ID NO: 166) linked by a peptidic spacer. The single-chain meganuclease is able to cleave a chimeric DNA target sequence comprising one different half of each parent meganuclease target sequence.

by “DNA target”, “DNA target sequence”, “target sequence”, “target-site”, “target”, “site”; “site of interest”; “recognition site”, “recognition sequence”, “homing recognition site”, “homing site”, “cleavage site” is intended a 20 to 24 by double-stranded palindromic, partially palindromic (pseudo-palindromic) or non-palindromic polynucleotide sequence that is recognized and cleaved by a LAGLIDADG (SEQ ID NO: 166) homing endonuclease such as I-CreI, or a variant, or a single-chain chimeric meganuclease derived from I-CreI. These terms refer to a distinct DNA location, preferably a genomic location, at which a double stranded break (cleavage) is to be induced by the meganuclease. The DNA target is defined by the 5′ to 3′ sequence of one strand of the double-stranded polynucleotide, as indicated above for C1221. Cleavage of the DNA target occurs at the nucleotides at positions +2 and −2, respectively for the sense and the antisense strand. Unless otherwise indicated, the position at which cleavage of the DNA target by an I-Cre I meganuclease variant occurs, corresponds to the cleavage site on the sense strand of the DNA target.

by “DNA target half-site”, “half cleavage site” or half-site” is intended the portion of the DNA target which is bound by each LAGLIDADG homing endonuclease core domain (SEQ ID NO: 166).

by “chimeric DNA target” or “hybrid DNA target” is intended the fusion of a different half of two parent meganuclease target sequences. In addition at least one half of said target may comprise the combination of nucleotides which are bound by at least two separate subdomains (combined DNA target).

by “human IL2RG gene” is intended the normal (wild-type IL2RG) located on chromosome X (Xq13.1; Gene ID: 3561) and the mutated IL2RG genes (mutant IL2RG; IL2RG allele), in particular the mutants responsible for SCID-X1. The human IL2RG gene (4145 bp) corresponds to positions 70243984 to 70248128 on the reverse complement strand of the sequence accession number GenBank NC_{—}000023.9. It comprises eight exons (Exon 1: positions 1 to 129; Exon 2: positions 504 to 657; Exon 3: positions 866 to 1050; Exon 4: positions 1259 to 1398; Exon 5: positions 2164 to 2326; Exon 6: positions 2859 to 2955; Exon 7: positions 3208 to 3277; Exon 8: positions 3633 to 4145). The ORF which is from position 15 (Exon 1) to position 3818 (Exon 8), is flanked by short and long untranslated regions, respectively at the 5′ and 3′ end. The wild-type IL2RG gene sequence corresponds to SEQ ID NO: 3 in the sequence listing; the mRNA sequence corresponds to GenBank NM_{—}000206 (SEQ ID NO: 112) and the gamma C receptor amino acid sequence to GenBank NP_{—}000197 (SEQ ID NO: 113). The mature protein (347 amino acids) is derived from a 369 amino acid precursor comprising a 22 amino acid N-terminal signal peptide.

by “DNA target sequence from the IL2RG gene”, “genomic DNA target sequence”, “genomic DNA cleavage site”, “genomic DNA target” or “genomic target” is intended a 20 to 24 by sequence of a primate (simian) IL2RG gene locus, for example the human IL2RG gene locus, which is recognized and cleaved by a meganuclease variant or a single-chain chimeric meganuclease derivative.

by “vector” is intended a nucleic acid molecule capable of transporting another nucleic acid to which it has been linked.

by “homologous” is intended a sequence with enough identity to another one to lead to a homologous recombination between sequences, more particularly having at least 95% identity, preferably 97% identity and more preferably 99%.

“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default settings.

“individual” includes mammals, as well as other vertebrates (e.g., birds, fish and reptiles). The terms “mammal” and “mammalian”, as used herein, refer to any vertebrate animal, including monotremes, marsupials and placental, that suckle their young and either give birth to living young (eutharian or placental mammals) or are egg-laying (metatharian or nonplacental mammals). Examples of mammalian species include humans and other primates (e.g., monkeys, chimpanzees), rodents (e.g., rats, mice, guinea pigs) and others such as for example: cows, pigs and horses.

by mutation is intended the substitution, deletion, insertion of one or more nucleotides/amino acids in a polynucleotide (cDNA, gene) or a polypeptide sequence. Said mutation can affect the coding sequence of a gene or its regulatory sequence. It may also affect the structure of the genomic sequence or the structure/stability of the encoded mRNA.

The variant according to the present invention may be a homodimer or a heterodimer. Preferably, both monomers of the heterodimer are mutated at positions 26 to 40 and/or 44 to 77. More preferably, both monomers have different substitutions both at positions 26 to 40 and 44 to 77 of I-CreI.

In a preferred embodiment of said variant, said substitution(s) in the subdomain situated from positions 44 to 77 of I-CreI are at positions 44, 68, 70, 75 and/or 77.

In another preferred embodiment of said variant, said substitution(s) in the subdomain situated from positions 26 to 40 of I-CreI are at positions 26, 28, 30, 32, 33, 38 and/or 40.

In another preferred embodiment of said variant, it comprises one or more mutations at positions of other amino acid residues that contact the DNA target sequence or interact with the DNA backbone or with the nucleotide bases, directly or via a water molecule; these residues are well-known in the art (Jurica et al., Molecular Cell., 1998, 2, 469-476; Chevalier et al., J. Mol. Biol., 2003, 329, 253-269). In particular, additional substitutions may be introduced at positions contacting the phosphate backbone, for example in the final C-terminal loop (positions 137 to 143; Prieto et al., Nucleic Acids Res., Epub 22 April 2007). Preferably said residues are involved in binding and cleavage of said DNA cleavage site. More preferably, said residues are at positions 138, 139, 142 or 143 of I-CreI. Two residues may be mutated in one variant provided that each mutation is in a different pair of residues chosen from the pair of residues at positions 138 and 139 and the pair of residues at positions 142 and 143. The mutations which are introduced modify the interaction(s) of said amino acid(s) of the final C-terminal loop with the phosphate backbone of the I-CreI site. Preferably, the residue at position 138 or 139 is substituted by an hydrophobic amino acid to avoid the formation of hydrogen bonds with the phosphate backbone of the DNA cleavage site. For example, the residue at position 138 is substituted by an alanine or the residue at position 139 is substituted by a methionine. The residue at position 142 or 143 is advantageously substituted by a small amino acid, for example a glycine, to decrease the size of the side chains of these amino acid residues. More, preferably, said substitution in the final C-terminal loop modifies the specificity of the variant towards the nucleotide at positions ±1 to 2, ±6 to 7 and/or ±11 to 12 of the I-CreI site.

In another preferred embodiment of said variant, it comprises one or more additional mutations that improve the binding and/or the cleavage properties of the variant towards the DNA target sequence from the human IL2RG gene.

The additional residues which are mutated may be on the entire I-CreI sequence or in the C-terminal half of I-CreI (positions 80 to 163). Both I-CreI monomers are advantageously mutated; the mutation(s) in each monomer may be identical or different. For example, the variant comprises one or more additional substitutions at positions: 2, 4, 7, 8, 19, 24, 26, 31, 34, 39, 43, 50, 52, 54, 57, 59, 60, 64, 71, 79, 80, 82, 87, 89, 96, 98, 100, 103, 105, 107, 111, 117, 121, 122, 127, 129, 132, 135, 139, 140, 143, 147, 153, 154, 156, 157, 159, 160, 162 and 163. Said substitutions are advantageously selected from the group consisting of: N2D, K4E, K7E, E8G, G19S, I24V, I24T, Q26R, Q31R, K34R, L39I, F43L, F43I, Q50R, R52C, F54L, K57R, V59A, D60G, V64A, G71R, S79G, E80K, E80G, K82R, F87L, T89A, K96R, K98R, K100R, N103Y, N103D, V105A, K107R, K107E, Q111R, E117G, E117K, K121R, F122Y, T127N, V129A, I132V, I132T, L135Q, K139R, T140A, T143I, T147A, D153G, S154G, S156R, E157G, K159E, K159R, K160G, S162F, S162P and P163L. The variant may also comprise additional residues at the C-terminus. For example a glycine (G) and/or a proline (P) residue may be inserted at positions 164 and 165 of I-CreI, respectively. Preferably, the variant comprises at least one substitution selected from the group consisting of: G19S, I24V, F54L, E80K, F87L, V105A and I132V.

According to a more preferred embodiment of said variant, said additional mutation further impairs the formation of a functional homodimer. More preferably, said mutation is the G19S mutation. The G19S mutation is advantageously introduced in one of the two monomers of a heterodimeric I-CreI variant, so as to obtain a meganuclease having enhanced cleavage activity and enhanced cleavage specificity. In addition, to enhance the cleavage specificity further, the other monomer may carry a distinct mutation that impairs the formation of a functional homodimer or favors the formation of the heterodimer.

In another preferred embodiment of said variant, said substitutions are replacement of the initial amino acids with amino acids selected from the group consisting of: A, D, E, G, H, K, N, P, Q, R, S, T, Y, C, V, L and W.

The variant of the invention may be derived from the wild-type I-CreI (SEQ ID NO: 1) or an I-CreI scaffold protein having at least 85% identity, preferably at least 90% identity, more preferably at least 95% identity with SEQ ID NO: 1, such as the scaffold I-CreI N75 (SEQ ID NO: 4; 167 amino acids) having the insertion of an alanine at position 2, and the insertion of AAD at the C-terminus (positions 164 to 166) of the I-CreI sequence.

The variant of the invention may include one or more residues inserted at the NH₂ terminus and/or COOH terminus of the sequence. For example, the variant may have the AAD or GPD sequence inserted at its C-terminus. In particular, a tag (epitope or polyhistidine sequence) may be introduced at the NH₂ terminus and/or COOH terminus; said tag is useful for the detection and/or the purification of said variant. The variant may also comprise a nuclear localization signal (NLS); said NLS is useful for the importation of said variant into the cell nucleus. The NLS may be inserted just after the first methionine of the variant or just after an N-terminal tag.

The variant according to the present invention may be a homodimer which is able to cleave a palindromic or pseudo-palindromic DNA target sequence.

Alternatively, said variant is a heterodimer, resulting from the association of a first and a second monomer having different substitutions at positions 26 to 40 and 44 to 77 of I-CreI, said heterodimer being able to cleave a non-palindromic DNA target sequence from the human IL2RG gene.

Each monomer (first monomer and second monomer) of the heterodimeric variant according to the present invention may be named with a letter code, after the eleven residues at positions 28, 30, 32, 33, 38, 40 and 44, 68, 70, 75, 77 and the additional residues which are mutated, as indicated above. For example, KNSSRE/LRNNI+80K or 28K30N32S33S38R40E/44L68R70N75N771+80K stands for I-CreI K28, N30, S32, S33, R38, E40/L44, R68, N70, N75, 177 and K80. I-CreI has K, N, S, Y, Q, S, Q, R, R, D and I, at positions 28, 30, 32, 33, 38, 40, 44, 68, 70, 75 and 77 respectively (KNSYQS/QRRDI). Therefore, KNSSRE/LRNNI+80K differs from I-CreI by at least the following substitutions: Y33S, Q38R, S40E, Q44L, R7ON, D75N and E80K.

The DNA target sequence which is cleaved by said variant may be in an exon or in an intron of the human IL2RG gene. In another preferred embodiment of said variant, said DNA target sequence is selected from the group consisting of the sequences SEQ ID NO: 5 to 9 and 116 to 119 (FIG. 3 and Table I).

TABLE I
Human IL2RG gene target sequences

| SEQ ID NO: | Sequence | Position* | Name | Location |
|---|---|---|---|---|
| 5 | ctcactatgttgcctaggctgg | 339 | IL2RG7 | Intron 1 |
| 116 | agggatactgtgggacattgga | 1080 | IL2RG13 | Intron 3 |
| 117 | gatcctgacttgtctaggccag | 1205 | IL2RG14 | Intron 3 |
| 6 | ctcactctgttgcccaggcttg | 1635 | IL2RG4 | Intron 4 |
| 7 | cgacctctgtctccctggttca | 1686 | IL2RG3 | Intron 4 |
| 118 | ttgcctagtgtggatgggcaga | 2197 | IL2RG12 | Exon 5 |
| 8 | tggaacggtgagatttggagaa | 2949 | IL2RG5 | Exon 6 |
| 119 | gaagcccagaaaaatgagggga | 2968 | IL2RG15 | Intron 6 |
| 9 | tcatatgggacaactgggagaa | 3130 | IL2RG6 | Intron 6 |

*the indicated position which is that of the first nucleotide of the target is indicated by reference to the human IL2RG gene sequence (SEQ ID NO: 3)

More preferably, for cleaving the IL2RG7 target that is located in Intron 1 of the human IL2RG gene (FIG. 3 and Table I), the monomers of the I-CreI variant have at least the following substitutions, respectively for the first and the second I-CreI monomer: Y33T, S40Q, Q44N, R68Y, R70S, D75Y and I77Q (or KNSTQQ/NYSYQ; first monomer), and K28S, Q38R, S40K, Q44D, R68N, R70S and D75N (or SNSYRK/DNSNI; second monomer).

More preferably, for cleaving the IL2RG13 target that is located in Intron 3 of the human IL2RG gene (FIG. 3 and Table I), the monomers of the I-CreI variant have at least the following substitutions, respectively for the first and the second I-CreI monomer: N30H, S32T, Y33C, Q38R, R70D, D75N and I77R (or KHTCRS/QRDNR; first monomer), and S32D, Q38Y, R70S, D75H and I77Y (or KNDYYS/QRSHY; second monomer).

More preferably, for cleaving the the IL2RG14 target that is located in Intron 3 of the human IL2RG gene (FIG. 3 and Table I) the monomers of the I-CreI variant have at least the following substitutions, respectively for the first and the second I-CreI monomer: N30R, S32A, Y33N, S40E, Q44Y, R70S and D75Q (or KRANQE/YRSQI; first monomer), and Y33C, Q38A, Q44N, R70S, D75Y and I77N (or KNSCAS/NRSYN; second monomer).

More preferably, for cleaving the IL2RG4 target that is located in Intron 4 of the human IL2RG (FIG. 3 and Table I), the monomers of the I-CreI variant have at least the following substitutions, respectively for the first and the second I-CreI monomer: Y33T, S40Q, Q44R, R68Y, R70S, D75E and I77Y (or KNSTQQ/RYSEY; first monomer), and S32T, Q44D, R68Y, R70S, D75S and I77R (or KNTYQS/DYSSR; second monomer).

More preferably, for cleaving the IL2RG3 target that is located in Intron 4 of the human IL2RG gene (FIG. 3 and Table I), the I-CreI variant has at least the following substitutions, respectively for the first and the second I-CreI monomer:

a first monomer having K at position 28, N at position 30, S at position 32, H or R at position 33, Q at position 38, Y or S at position 40, K or R at position 44, Y at position 68, S at position 70, D or E at position 75 and T or V at position 77. Preferably, the residues at positions 28, 30, 32, 33, 38 and 40 are selected from the group consisting of: KNSRQY, KNSHQS, KNSRQS, KNSHQY and KNSRQY, and the residues at positions 44, 68, 70, 75 and 77 are selected from the group consisting of: RYSDT, KYSEV or RYSEV. More preferably, the first monomer is selected from the group consisting of: KNSRQY/RYSDT, KNSHQS/KYSEV, KNSRQS/RYSDT, KNSHQY/RYSDT, KNSHQY/KYSEV, KNSRQY/RYSEV, and KNSHQY/RYSEV. The first monomer comprises advantageously at least one first additional mutation selected from the group consisting of: G19S, F54L, F87L, V105A and I132V, and eventually a second additional mutation selected from the group consisting of: N2D, K4E, K7E, E8G, Q26R, Q31R, K34R, L39I, F43L, G71R, E80G, K82R, T89A, Q111R, E117G, K121R, T127N, I132T, K139R, T143I, T147A, S154G, E157G, K159E, K159R, K160G, S162F, S162P and P163L. Examples of such first monomers are presented in Table VI (m10: Y33R, S40Y, Q44R, R68Y, R70S, I77T and I132V or KNSRQY/RYSDT+132V, corresponding to SEQ ID NO: 40), Table VII (SEQ ID NO: 67 to 72), Table VIII (.3R_{—}1 to .3R_{—}11, corresponding to SEQ ID NO: 73 to 83), Table IX (.3R_{—}12 to .3R_{—}28, corresponding to SEQ ID NO: 84 to 100) and Table XIV (.3R_{—}25a, .3R_{—}25b and .3R_{—}25c, corresponding to SEQ ID NO: 140 to 142). Preferred first monomers are .3R_{—}17, .3R_{—}27, 3R_{—}28, .3R_{—}25a and 3R_{—}25c, corresponding to SEQ ID NO: 89, 99, 100, 140 and 142, respectively.

a second monomer having K at position 28, R or Nat position 30, S, G or T at position 32, Y, N, A, V, S or H at position 33, Q at position 38, S at position 40, A, T or R at position 44, R or Y at position 68, S at position 70, E at position 75 and R at position 77. Preferably, the residues at positions 28, 30, 32, 33, 38 and 40 are selected from the group consisting of: KRTYQS, KRSYQS, KRSNQS, KRSAQS, KRSVQS, KRSSQS and KNGHQS and the residues at positions 44, 68, 70, 75 and 77 are selected from the group consisting of: AYSER, TRSER, TYSER, and RYSET. More preferably, the second monomer is selected from the group consisting of: KRTYQS/AYSER, KRSYQS/TRSER, KRSNQS/TYSER, KRSAQS/TRSER, KRSVQS/TRSER, KRSSQS/RYSET and KNGHQS/TRSER. The second monomer comprises advantageously at least one first additional mutation selected from the group consisting of: G19S, I24V, F54L, E80K, F87L, V105A and I132V, and eventually a second additional mutation selected from the group consisting of: I24T, Q31R, K34R, F43L, F43I, Q50R, R52C, K57R, V59A, D60G, V64A, K82R, K96R, K98R, K100R, N103Y, N103D, K107R, K107E, Q111R, E117K, F122Y, V129A, L135Q, T140A, D153G and S156R. Examples of such second monomers are presented in Table VI (M1: N30R, S32T, Q44A, R68Y, R70S, D75E and I77R or KRTYQS/AYSER, corresponding to SEQ ID NO: 45), Table X (.4_R0 to .4_R3, corresponding to SEQ ID NO: 101 to 104), Table XI (.4_R4 to .4_R6 and 4_R8 to .4_R11, corresponding to SEQ ID NO: 105 to 107 and 108 to 111), Table XIII (SEQ ID No: 128 to 139), Table XV (SEQ ID NO: 143 to 148), Table XVI (SEQ ID NO: 156 to 162) and Table XVII (SEQ ID NO: 163 to 165). Preferred second monomers are .4_R2, 4_R5, 4_R9, 4_R11, M1_{—}24V and its derived mutants of Table XV, corresponding to SEQ ID NO: 103, 106, 109, 111, 128 and 143 to 148, respectively.

More preferably, for cleaving the IL2RG12 target that is located in Exon 5 of the human IL2RG gene (FIG. 3 and Table I), the monomers of the I-CreI variant have at least the following substitutions, respectively for the first and the second I-CreI monomer: Y33S, Q38R, S40E, Q44L, R70N, D75N and E80K (or KNSSRE/LRNNI+E80K; first monomer), and N30D, Y33R, Q38T, Q44A, R68Y, R70S, D75Y and I77K (or KDSRTS/AYSYK; second monomer).

More preferably, for cleaving the IL2RG5 target that is located in Exon 6 of the human IL2RG gene (FIG. 3 and Table I), the monomers of the I-CreI variant have at least the following substitutions, respectively for the first and the second I-CreI monomer: Y33R, Q38N, S40Q, Q44Y, R70S and I77V (KNSRNQ/YRSDV; first monomer), and Y33T, Q38A, R68Y, R70S, D75R and I77Q (or KNSTAS/QYSRQ; second monomer).

More preferably, for cleaving the IL2RG15 target that is located in Intron 6 of the human IL2RG gene (FIG. 3 and Table I), the monomers of the I-CreI variant have at least the following substitutions, respectively for the first and the second I-CreI monomer:N30S, Y33C, R40A, Q44A, R68Y, R70S, D75Y and I77K (or KSSCQA/AYSYI; first monomer) and S32T, Q38W, Q44A, R68Y, R70S, D75Y and I77K (or KNTYWS/AYSYK; second monomer).

More preferably, for cleaving the IL2RG6 target that is located in Intron 6 of the human IL2RG gene (FIG. 3 and Table I), the monomers of the I-CreI variant have at least the following substitutions, respectively for the first and the second I-CreI monomer: S32R, Y33D, Q44D, R68N, R70S and D75N (or KNRDQS/DNSNI; first monomer), and Y33T, Q38A, Q44A, R68Y, R70S, D75Y, I77K (or KNSTAS/AYSYK; second monomer).

The heterodimeric variant as defined above may have only the amino acid substitutions as indicated above. In this case, the positions which are not indicated are not mutated and thus correspond to the wild-type I-CreI (SEQ ID NO: 1) or I-CreI N75 scaffold (SEQ ID NO: 4) sequence, respectively. Examples of such heterodimeric I-CreI variants cleaving the IL2RG DNA targets of Table I include the variants consisting of a first and a second monomer corresponding to the following pairs of sequences: SEQ ID NO: 38 and 43 (cleaving the IL2RG7 target); SEQ ID NO: 39 and 44 (cleaving the IL2RG4 target); SEQ ID NO: 40 (named m10) and SEQ ID NO: 45 (named M1), cleaving the IL2RG3 target; SEQ ID NO: 41 and SEQ ID NO: 46 (cleaving the IL2RG5 target); SEQ ID NO: 42 and SEQ ID NO: 47 (cleaving the IL2RG6 target); SEQ ID NO: 120 and 121 (IL2RG13), SEQ ID NO: 122 and 123 (IL2RG14), SEQ ID NO: 124 and 125 (IL2RG12) and SEQ ID NO: 126 and 127 (IL2RG15).

Alternatively, the heterodimeric variant may consist of an I-CreI sequence comprising the amino acid substitutions as defined above. In the latter case, the positions which are not indicated may comprise additional mutations, for example one or more additional mutations as defined above.

In particular, one or both monomers of the heterodimeric variant comprise advantageously additional substitutions that increase the cleavage activity of the variant for the IL2RG target.

For example, the monomers SEQ ID NO: 67 to 100, 140 to 142 and the monomers SEQ ID NO: 101 to 111, 128 to 139, 143 to 148 and 156 to 165 have additional substitutions that increase the cleavage activity for the IL2RG3 target.

Preferred heterodimeric variants cleaving the IL2RG3 target are:

KNSHQS/KYSEV+26R+31R+54L+139R (.3R_{—}17, corresponding to SEQ ID NO: 89; first monomer) and KRTYQS/AYSER+19S+59A+103Y+107R (.4R_{—}5, corresponding to SEQ ID NO: 106), KRTYQS/AYSER+19S+60G+156R (.4R_{—}9, corresponding to SEQ ID NO: 109) or KRTYQS/AYSER+24V (M1_{—}24V, corresponding to SEQ ID NO: 128; second monomer),

KNSHQS/KYSEV+31R+80G+132V+139R (.3R_{—}27, corresponding to SEQ ID NO: 99; first monomer) and KRTYQS/AYSER+19S+60G+156R (.4R_{—}9, corresponding to SEQ ID NO: 109) or KRTYQS/AYSER +19S+59A+82R+111R+140A (.4R_{—}11, corresponding to SEQ ID NO: 111; second monomer),

KNSHQS/KYSEV+31R+132V+139R (.3R_{—}28, corresponding to SEQ ID NO: 100; first monomer) and KRTYQS/AYSER+19S+59A+111R (.4R_{—}2, corresponding to SEQ ID NO: 103), KRTYQS/AYSER+19S+59A+103Y+107R (.4R_{—}5, corresponding to SEQ ID NO: 106), KRTYQS/AYSER+19S+60G+156R (.4R_{—}9, corresponding to SEQ ID NO: 109), KRTYQS/AYSER+19S+59A+82R+111R+140A (.4R_{—}11, corresponding to SEQ ID NO: 111) or KRTYQS/AYSER+24V (M1_{—}24V, corresponding to SEQ ID NO: 128; second monomer),

KNSHQY/RYSEV+19S+132V (.3_R25, corresponding to SEQ ID NO: 97) or KNSHQY/RYSEV+19S+71R+132V+139R (.3R_{—}25a, corresponding to SEQ ID NO: 140) or KNSHQY/RYSEV+19S+71R+132V (.3R_{—}25c, corresponding to SEQ ID NO: 142; first monomer) and KRTYQS/AYSER+24V (M1_{—}24V, corresponding to SEQ ID NO: 128; second monomer).

KNSHQS/KYSEV+26R+31R+54L+139R (.3R_{—}17, corresponding to SEQ ID NO: 89; first monomer) or KNSHQY/RYSEV+19S+132V (.3_R25, corresponding to SEQ ID NO: 97) and KRTYQS/AYSER+24V+132V (corresponding to SEQ ID NO: 143) or KRTYQS/AYSER+24V+80K (corresponding to SEQ ID NO: 144) or KRTYQS/AYSER+24V+54L (corresponding to SEQ ID NO: 145) or KRTYQS/AYSER+24V+87L (corresponding to SEQ ID NO: 146) or KRTYQS/AYSER+24V+105A (corresponding to SEQ ID NO: 147) or KRTYQS/AYSER+24V+105A+132V (corresponding to SEQ ID NO: 148).

The invention encompasses I-CreI variants having at least 85% identity, preferably at least 90% identity, more preferably at least 95% (96%, 97%, 98%, 99%) identity with the sequences as defined above, said variant being able to cleave a DNA target from the IL2RG gene.

The heterodimeric variant is advantageously an obligate heterodimer variant having at least one pair of mutations interesting corresponding residues of the first and the second monomers which make an intermolecular interaction between the two I-CreI monomers, wherein the first mutation of said pair(s) is in the first monomer and the second mutation of said pair(s) is in the second monomer and said pair(s) of mutations prevent the formation of functional homodimers from each monomer and allow the formation of a functional heterodimer, able to cleave the genomic DNA target from the human IL2RG gene.

To form an obligate heterodimer, the monomers have advantageously at least one of the following pairs of mutations, respectively for the first and the second monomer:

a) the substitution of the glutamic acid at position 8 with a basic amino acid, preferably an arginine (first monomer) and the substitution of the lysine at position 7 with an acidic amino acid, preferably a glutamic acid (second monomer); the first monomer may further comprise the substitution of at least one of the lysine residues at positions 7 and 96, by an arginine,

b) the substitution of the glutamic acid at position 61 with a basic amino acid, preferably an arginine (first monomer) and the substitution of the lysine at position 96 with an acidic amino acid, preferably a glutamic acid (second monomer); the first monomer may further comprise the substitution of at least one of the lysine residues at positions 7 and 96, by an arginine,

c) the substitution of the leucine at position 97 with an aromatic amino acid, preferably a phenylalanine (first monomer) and the substitution of the phenylalanine at position 54 with a small amino acid, preferably a glycine (second monomer); the first monomer may further comprise the substitution of the phenylalanine at position 54 by a tryptophane and the second monomer may further comprise the substitution of the leucine at position 58 or lysine at position 57, by a methionine, and

d) the substitution of the aspartic acid at position 137 with a basic amino acid, preferably an arginine (first monomer) and the substitution of the arginine at position 51 with an acidic amino acid, preferably a glutamic acid (second monomer).

For example, the first monomer may have the mutation D137R and the second monomer, the mutation R51D. The obligate heterodimer meganuclease comprises advantageously, at least two pairs of mutations as defined in a), b) c) or d), above; one of the pairs of mutation is advantageously as defined in c) or d). Preferably, one monomer comprises the substitution of the lysine residues at positions 7 and 96 by an acidic amino acid (aspartic acid (D) or glutamic acid (E)), preferably a glutamic acid (K7E and K96E) and the other monomer comprises the substitution of the glutamic acid residues at positions 8 and 61 by a basic amino acid (arginine (R) or lysine (K); for example, E8K and E61R). More preferably, the obligate heterodimer meganuclease, comprises three pairs of mutations as defined in a), b) and c), above. The obligate heterodimer meganuclease consists advantageously of (i) E8R, E8K or E8H, E61R, E61K or E61H and L97F, L97W or L97Y; (ii) K7R, E8R, E61R, K96R and L97F, or (iii) K7R, E8R, F54W, E61R, K96R and L97F and a second monomer (B) having at least the mutations (iv) K7E or K7D, F54G or F54A and K96D or K96E; (v) K7E, F54G, L58M and K96E, or (vi) K7E, F54G, K57M and K96E. For example, the first monomer may have the mutations K7R, E8R or E8K, E61R, K96R and L97F or K7R, E8R or E8K, F54W, E61R, K96R and L97F and the second monomer, the mutations K7E, F54G, L58M and K96E or K7E, F54G, K57M and K96E. The obligate heterodimer may comprise at least one NLS and/or one tag as defined above; said NLS and/or tag may be in the first and/or the second monomer

The subject-matter of the present invention is also a single-chain chimeric meganuclease (fusion protein) derived from an I-CreI variant as defined above. The single-chain meganuclease may comprise two I-CreI monomers, two I-CreI core domains (positions 6 to 94 of I-CreI) or a combination of both. Preferably, the two monomers/core domains or the combination of both, are connected by a peptidic linker.

The subject-matter of the present invention is also a polynucleotide fragment encoding a variant or a single-chain chimeric meganuclease as defined above; said polynucleotide may encode one monomer of a homodimeric or heterodimeric variant, or two domains/monomers of a single-chain chimeric meganuclease.

The subject-matter of the present invention is also a recombinant vector for the expression of a variant or a single-chain meganuclease according to the invention. The recombinant vector comprises at least one polynucleotide fragment encoding a variant or a single-chain meganuclease, as defined above. In a preferred embodiment, said vector comprises two different polynucleotide fragments, each encoding one of the monomers of a heterodimeric variant.

A vector which can be used in the present invention includes, but is not limited to, a viral vector, a plasmid, a RNA vector or a linear or circular DNA or RNA molecule which may consist of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e. g. adeno-associated viruses), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomegalovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosissarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).

Preferred vectors include lentiviral vectors, and particularly self inactivacting lentiviral vectors.

Vectors can comprise selectable markers, for example: neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1, URA3 and LEU2 for S. cerevisiae; tetracycline, rifampicin or ampicillin resistance in E. coli.

Preferably said vectors are expression vectors, wherein the sequence(s) encoding the variant/single-chain meganuclease of the invention is placed under control of appropriate transcriptional and translational control elements to permit production or synthesis of said variant. Therefore, said polynucleotide is comprised in an expression cassette. More particularly, the vector comprises a replication origin, a promoter operatively linked to said encoding polynucleotide, a ribosome-binding site, an RNA-splicing site (when genomic DNA is used), a polyadenylation site and a transcription termination site. It also can comprise an enhancer. Selection of the promoter will depend upon the cell in which the polypeptide is expressed. Preferably, when said variant is a heterodimer, the two polynucleotides encoding each of the monomers are included in one vector which is able to drive the expression of both polynucleotides, simultaneously. Suitable promoters include tissue specific and/or inducible promoters. Examples of inducible promoters are: eukaryotic metallothionine promoter which is induced by increased levels of heavy metals, prokaryotic lacZ promoter which is induced in response to isopropyl-β-D-thiogalacto-pyranoside (IPTG) and eukaryotic heat shock promoter which is induced by increased temperature. Examples of tissue specific promoters are skeletal muscle creatine kinase, prostate-specific antigen (PSA), α-antitrypsin protease, human surfactant (SP) A and B proteins, β-casein and acidic whey protein genes.

According to another advantageous embodiment of said vector, it includes a targeting construct comprising sequences sharing homologies with the region surrounding the genomic DNA cleavage site as defined above.

Preferably, said sequence sharing homologies with the regions surrounding the genomic DNA cleavage site of the variant is a fragment of the human IL2RG gene comprising positions: 250 to 449, 991 to 1190, 1116 to 1305, 1546 to 1745, 1597 to 1796, 2108 to 2307, 2860 to 3059, 2879 to 3078 or 3041 to 3240 of SEQ ID NO: 3.

Alternatively, the vector coding for an I-CreI variant/single-chain meganuclease and the vector comprising the targeting construct are different vectors.

More preferably, the targeting DNA construct comprises:

a) sequences sharing homologies with the region surrounding the genomic DNA cleavage site as defined above, and

b) a sequence to be introduced flanked by sequences as in a) or included in sequences as in a).

Preferably, homologous sequences of at least 50 bp, preferably more than 100 by and more preferably more than 200 by are used. Therefore, the targeting DNA construct is preferably from 200 pb to 6000 pb, more preferably from 1000 pb to 2000 pb. Indeed, shared DNA homologies are located in regions flanking upstream and downstream the site of the break and the DNA sequence to be introduced should be located between the two arms. The sequence to be introduced is preferably a sequence which repairs a mutation in the gene of interest (gene correction or recovery of a functional gene), for the purpose of genome therapy. Alternatively, it can be any other sequence used to alter the chromosomal DNA in some specific way including a sequence used to modify a specific sequence, to attenuate or activate the gene of interest, to inactivate or delete the gene of interest or part thereof, to introduce a mutation into a site of interest or to introduce an exogenous gene or part thereof. Such chromosomal DNA alterations are used for genome engineering (animal models/human recombinant cell lines). The targeting construct comprises advantageously a positive selection marker between the two homology arms and eventually a negative selection marker upstream of the first homology arm or downstream of the second homology arm. The marker(s) allow(s) the selection of cells having inserted the sequence of interest by homologous recombination at the target site.

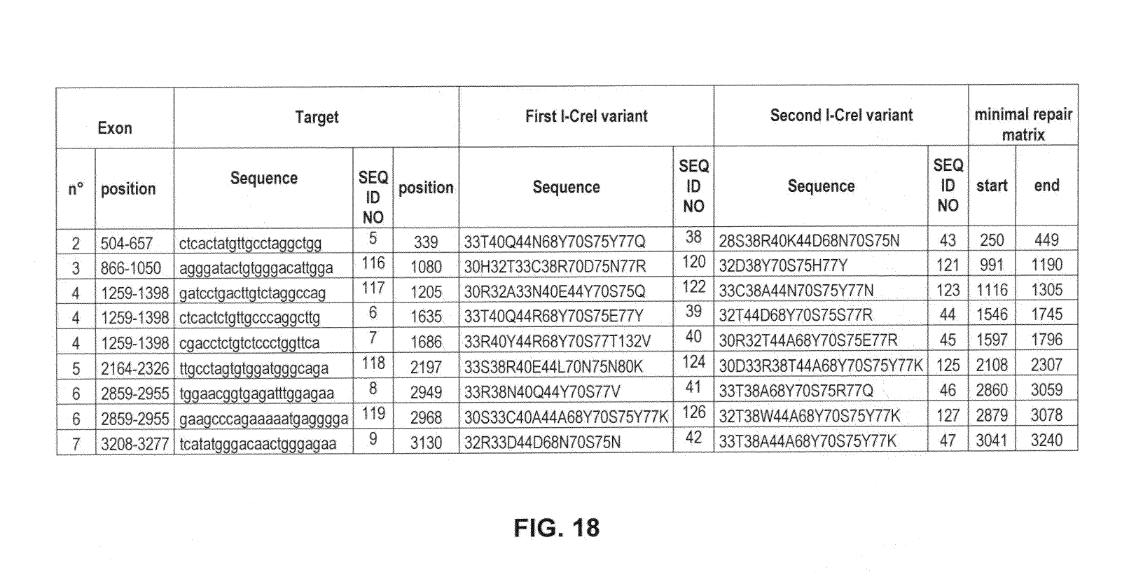

For example, FIG. 18 indicates the targets from the IL2RG gene, variants which are able to cleave said targets and the minimal matrix for repairing the cleavage at each target site.

For correcting the IL2RG gene, cleavage of the gene occurs in the vicinity of the mutation, preferably, within 500 by of the mutation (FIG. 1A). The targeting construct comprises a IL2RG gene fragment which has at least 200 by of homologous sequence flanking the target site (minimal repair matrix) for repairing the cleavage, and includes a sequence encoding a portion of wild-type IL2RG chain corresponding to the region of the mutation for repairing the mutation (FIG. 1A). Consequently, the targeting construct for gene correction comprises or consists of the minimal repair matrix; it is preferably from 200 pb to 6000 pb, more preferably from 1000 pb to 2000 pb. Preferably, when the cleavage site of the variant overlaps with the mutation, the repair matrix includes a modified cleavage site that is not cleaved by the variant which is used to induce said cleavage in the IL2RG gene and a sequence encoding wild-type human IL2RG chain that does not change the open reading frame of the human IL2RG chain.

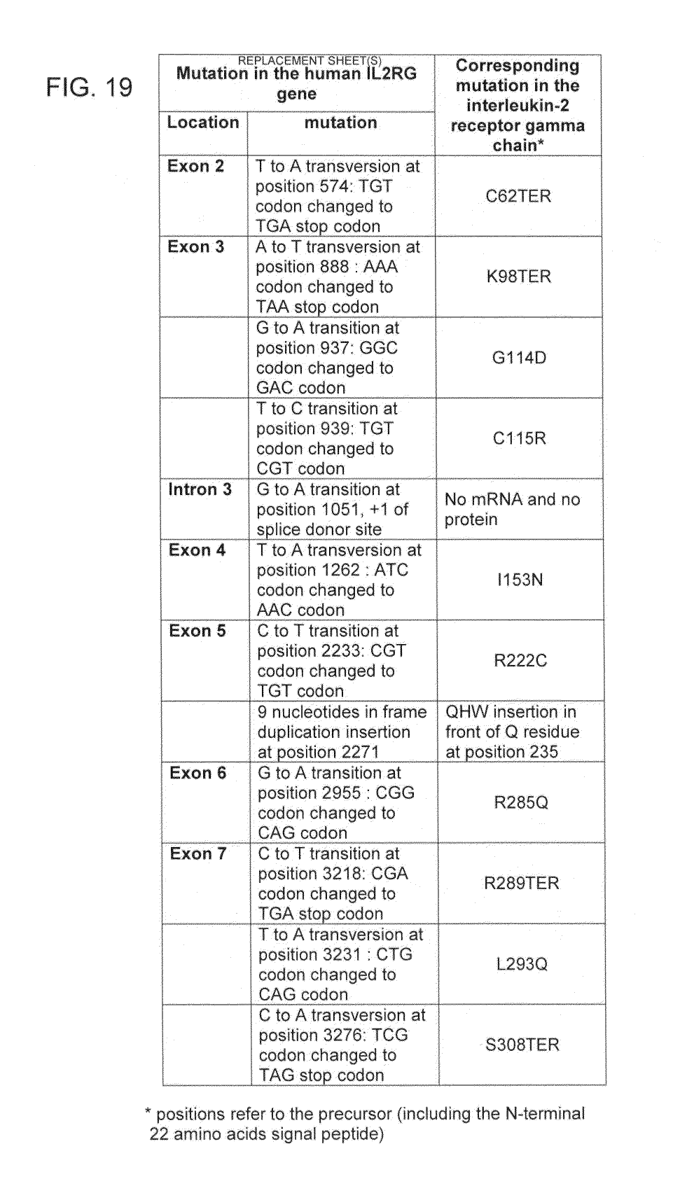

For example, for correcting some of the mutations in the IL2RG gene responsible for SCID-X1, as indicated in FIG. 19, the following combinations of variants/targeting constructs may be used:

C62TER (Exon 2)

Y33T, S40Q, Q44N, R68Y, R70S, D75Y and I77Q (or KNSTQQ/NYSYQ; first monomer), and K28S, Q38R, S40K, Q44D, R68N, R70S and D75N (or SNSYRK/DNSNI; second monomer) which cleaves the IL2RG7 target that is located in Intron 1 of the human IL2RG gene (FIGS. 3 and 18), and a targeting construct comprising at least positions 250 to 449 of the human IL2RG gene for efficient repair of the DNA double-strand break, and all sequences between the meganuclease cleavage site (at position 351) and the mutation site (at position 574), for efficient repair of the mutation. An example of variant is the heterodimer formed of SEQ ID NO: 38 and SEQ ID NO: 43.

K98TER, G114D, C115R (Exon 3), Null Mutation (Intron 3)

N30H, S32T, Y33C, Q38R, R70D, D75N and I77R (or KHTCRS/QRDNR; first monomer), and S32D, Q38Y, R70S, D75H and I77Y (or KNDYYS/QRSHY; second monomer) which cleaves the IL2RG13 target that is located in Intron 3 of the human IL2RG gene (FIG. 3 and Table I), and a targeting construct comprising at least positions 991 to 1190 of the human IL2RG gene for efficient repair of the DNA double-strand break, and all sequences between the meganuclease cleavage site (at position 1092) and the mutation site (at position 888 (K98TER), 937 (G114D), 939 (C115R) or 1051 (null mutation)), for efficient repair of the mutation. An example of variant is the heterodimer formed of SEQ ID NO: 120 and SEQ ID NO: 121.

N30R, S32A, Y33N, S40E, Q44Y, R70S and D75Q (or KRANQE/YRSQI; first monomer), and Y33C, Q38A, Q44N, R705, D75Y and I77N (or KNSCAS/NRSYN; second monomer) which cleaves the IL2RG14 target that is located in Intron 3 of the human IL2RG gene (FIG. 3 and Table I), and a targeting construct comprising at least positions 1116 to 1305 of the human IL2RG gene for efficient repair of the DNA double-strand break, and all sequences between the meganuclease cleavage site (at position 1217) and the mutation site (at position 888 (K98TER), 937 (G114D), 939 (C115R) or 1051 (null mutation)), for efficient repair of the mutation. An example of variant is the heterodimer formed of SEQ ID NO: 122 and SEQ ID NO: 123.

I153N (Exon 4)

Y33T, S40Q, Q44R, R68Y, R70S, D75E and I77Y (or KNSTQQ/RYSEY; first monomer), and S32T, Q44D, R68Y, R70S, D75S and I77R (or KNTYQS/DYSSR; second monomer) which cleaves the IL2RG4 target that is located in Intron 4 of the human IL2RG (FIGS. 3 and 18), and a targeting construct comprising at least positions 1546 to 1745 of the human IL2RG gene for efficient repair of the DNA double-strand break, and all sequences between the meganuclease cleavage site (at position 1647) and the mutation site (at position 1262), for efficient repair of the mutation. An example of variant is the heterodimer formed of SEQ ID NO: 39 and SEQ ID NO: 44.

Y33R, S40Y, Q44R, R68Y, R70S, I77T and I132V (or KNSRQY/RYSNT+I132V; first monomer), and N30R, S32T, Q44A, R68Y, R70S, D75E and I77R (or KRTYQS/AYSER; second monomer) which cleaves the IL2RG3 target that is located in Intron 4 of the human IL2RG gene (FIGS. 3 and 18), and a targeting construct comprising at least positions 1597 to 1796 of the human IL2RG gene for efficient repair of the DNA double-strand break, and all sequences between the meganuclease cleavage site (at position 1698) and the mutation site (at position 1262), for efficient repair of the mutation. Examples of variants are the heterodimer formed of SEQ ID NO: 40 (m10) and SEQ ID NO: 45 (M1) and the derived heterodimers formed of monomers having additional substitutions that increase the cleavage activity for the IL2RG3 target: SEQ ID NO: 67 to 100, 140 to 142 (first monomer) and SEQ ID NO: 101 to 111, 128 to 139 and 143 to 148 (second monomer derived from M1). Preferred heterodimers are SEQ ID NO: 89 and any of SEQ ID NO: 106, 109, 128; SEQ ID NO: 99 and SEQ ID NO: 109 or 111; SEQ ID NO: 100 and any of SEQ ID NO: 103, 106, 109, 111 and 128; SEQ ID NO: 140 or 142 and SEQ ID NO: 128.

R222C and QHW Insertion in Front of Q 235 (Exon 5)

Y33S, Q38R, S40E, Q44L, R70N, D75N and E80K (or KNSSRE/LRNNI+E80K; first monomer), and N30D, Y33R, Q38T, Q44A, R68Y, R70S, D75Y and I77K (or KDSRTS/AYSYK; second monomer) which cleaves the IL2RG12 target that is located in Exon 5 of the human IL2RG gene (FIG. 3 and Table I), and a targeting construct comprising at least positions 2108 to 2307 of the human IL2RG gene for efficient repair of both the DNA double-strand break and the mutation. This targeting construct comprises all the sequences between the meganuclease cleavage site (at position 2209) and the mutation site (at position 2233 (R222C) or 2271 (QHW insertion), for efficient repair of the mutation. An example of variant is the heterodimer formed of SEQ ID NO: 124 and SEQ ID NO: 125.

R285Q (Exon 6), R289TER, L293Q and S308TER (Exon 7)

Y33R, Q38N, S40Q, Q44Y, R70S and I77V (or KNSRNQ/YRSDV; first monomer), and Y33T, Q38A, R68Y, R70S, D75R and I77Q (or KNSTAS/QYSRQ; second monomer) which cleaves the IL2RG5 target that is located in Exon 6 of the human IL2RG gene (FIGS. 3 and 18), and a targeting construct comprising at least positions 2860 to 3059 of the human IL2RG gene for efficient repair of the DNA double-strand break, and all sequences between the meganuclease cleavage site (at position 2961) and the mutation site (at positions 2955(R285Q), 3218(R289TER), 3231 (L293Q) or 3276 (S308TER)) for efficient repair of the mutation. An example of variant is the heterodimer formed of SEQ ID NO: 41 and SEQ ID NO: 46.

S32R, Y33D, Q44D, R68N, R70S and D75N (or KNRDQS/DNSNI; first monomer), and Y33T, Q38A, Q44A, R68Y, R70S, D75Y, I77K (or KNSTAS/AYSYK; second monomer) which cleaves the IL2RG6 target that is located in Intron 6 of the human IL2RG gene (FIGS. 3 and 18) and a targeting construct comprising at least positions 3041 to 3240 of the human IL2RG gene for efficient repair of the DNA double-strand break, and all sequences between the meganuclease cleavage site (at position 3142) and the mutation site (at positions 2955(R285Q), 3218(R289TER), 3231 (L293Q) or 3276 (S308TER)) for efficient repair of the mutation. An example of variant is the heterodimer formed of SEQ ID NO: 42 and SEQ ID NO: 47.

Y33R, Q38N, S40Q, Q44Y, R70S and I77V (KNSRNQ/YRSDV; first monomer), and Y33T, Q38A, R68Y, R70S, D75R and I77Q (or KNSTAS/QYSRQ; second monomer) which cleaves the IL2RG5 target that is located in Exon 6 of the human IL2RG gene (FIG. 3 and Table I), and a targeting construct comprising at least positions 2879 to 3078 of the human IL2RG gene for efficient repair of the DNA double-strand break, and all sequences between the meganuclease cleavage site (at position 2980) and the mutation site (at positions 3218(R289TER), 3231 (L293Q) or 3276 (S308TER)), for efficient repair of the mutation. This targeting construct comprises all the sequences between the meganuclease cleavage site (at position 2980) and the mutation site at position 2955(R285Q), for efficient repair of this mutation. An example of variant is the heterodimer formed of SEQ ID NO: 126 and SEQ ID NO: 127.

Alternatively, for restoring a functional gene (FIG. 1B), cleavage of the gene occurs upstream of a mutation. Preferably said mutation is the first known mutation in the sequence of the gene, so that all the downstream mutations of the gene can be corrected simultaneously. The targeting construct comprises the exons downstream of the cleavage site fused in frame (as in the cDNA) and with a polyadenylation site to stop transcription in 3′. The sequence to be introduced (exon knock-in construct) is flanked by introns or exons sequences surrounding the cleavage site, so as to allow the transcription of the engineered gene (exon knock-in gene) into a mRNA able to code for a functional protein (FIG. 1B). For example, the exon knock-in construct is flanked by sequences upstream and downstream of the cleavage site, from a minimal repair matrix as defined above. Therefore, cleavage occurs preferably in Intron 1 (IL2RG7 target) with the variant described aboveThe variant cleaving the IL2RG7 target may be used with a targeting construct comprising Exon 1 to 8 fused in frame (as in the cDNA) and with a polyadenylation site to stop transcription in 3′ and is terminated by sequences downstream of the cleavage site. Alternatively, cleavage occurs in Intron 4 (IL2RG3 or IL2RG4 target) with the variants described above. The variants cleaving IL2RG3 or IL2RG4 may be used with a targeting construct comprising Exons 5 to 8 fused in frame (as in the cDNA) and with a polyadenylation site to stop transcription in 3′, flanked by exon and intron sequences surrounding the cleavage site, so as to allow the transcription of the engineered gene (exon knock-in gene) into a mRNA able to code for a functional protein (FIG. 1B).

For making knock-in animals/cells, the targeting DNA construct comprises: a human IL2RG gene fragment which has at least 200 by of homologous sequence flanking the target site for repairing the cleavage, the sequence of an exogeneous gene of interest, and eventually a selection marker, such as the neomycin gene.

For the insertion of a sequence, DNA homologies are generally located in regions directly upstream and downstream to the site of the break (sequences immediately adjacent to the break; minimal repair matrix). However, when the insertion is associated with a deletion of ORF sequences flanking the cleavage site, shared DNA homologies are located in regions upstream and downstream the region of the deletion.

The modification(s) in the human IL2RG gene are introduced in human cells, for the purpose of human genome therapy or the making of human recombinant cell lines. However they may also be introduced in humanized cells wherein the endogenous IL2RG gene has been deleted (knock-out) and a normal or mutated human IL2RG gene has been introduced anywhere in the genome (transgenic) or specifically at the endogenous IL2RG locus (knock-in), for the purpose of making animal models of SCID-X1 or studying the correction of the mutation by meganuclease-induced homologous recombination.

The subject matter of the present invention is also a targeting DNA construct as defined above.

The subject-matter of the present invention is also a composition characterized in that it comprises at least one meganuclease as defined above (variant or single-chain derived chimeric meganuclease) and/or at least one expression vector encoding said meganuclease, as defined above.

In a preferred embodiment of said composition, it comprises a targeting DNA construct, as defined above.

Preferably, said targeting DNA construct is either included in a recombinant vector or it is included in an expression vector comprising the polynucleotide(s) encoding the meganuclease according to the invention.

The subject-matter of the present invention is also the use of at least one meganuclease and/or one expression vector, as defined above, for the preparation of a medicament for preventing, improving or curing X-linked severe combined immunodeficiency (SCID-X1), in an individual in need thereof.

The use of the meganuclease may comprise at least the step of (a) inducing in somatic tissue(s) of the donor/individual a double stranded cleavage at a site of interest of the IL2RG gene comprising at least one recognition and cleavage site of said meganuclease by contacting said cleavage site with said meganuclease, and (b) introducing into said somatic tissue(s) a targeting DNA, wherein said targeting DNA comprises (1) DNA sharing homologies to the region surrounding the cleavage site and (2) DNA which repairs the IL2RG gene upon recombination between the targeting DNA and the chromosomal DNA, as defined above. The targeting DNA is introduced into the somatic tissues(s) under conditions appropriate for introduction of the targeting DNA into the site of interest.

According to the present invention, said double-stranded cleavage may be induced, ex vivo by introduction of said meganuclease into somatic cells (hematopietic stem cells) from the diseased individual and then transplantation of the modified cells back into the diseased individual. The targeting construct may comprise sequences for deleting the human IL2RG gene and eventually the sequence of an exogenous gene of interest (gene replacement).

The subject-matter of the present invention is also a method for preventing, improving or curing X-linked severe combined immunodeficiency (SCID-X1) in an individual in need thereof, said method comprising at least the step of administering to said individual a composition as defined above, by any means.

The subject-matter of the present invention is further the use of a meganuclease as defined above or one or two polynucleotide(s), preferably included in expression vector(s), for genome engineering of the IL2RG gene, for non-therapeutic purposes. The IL2RG gene may be the human endogenous IL2RG gene (human IL2RG gene locus; human recombinant cells generation) or a transgene that has been inserted in an animal, for example a mouse (animal models of SCID-X1).

According to an advantageous embodiment of said use, it is for inducing a double-strand break in a site of interest of the IL2RG gene comprising a genomic DNA target sequence, thereby inducing a DNA recombination event, a DNA loss or cell death.

According to the invention, said double-strand break is for: repairing a specific sequence in the human IL2RG gene, modifying a specific sequence in the human IL2RG gene, restoring a functional human IL2RG gene in place of a mutated one, attenuating or activating the human IL2RG gene, introducing a mutation into a site of interest of the human IL2RG gene, introducing an exogenous gene or a part thereof, inactivating or deleting the human IL2RG gene or a part thereof, translocating a chromosomal arm, or leaving the DNA unrepaired and degraded.

According to another advantageous embodiment of said use, said variant, polynucleotide(s), or vector, are associated with a targeting DNA construct as defined above.

In a first embodiment of the use of the meganuclease according to the present invention, it comprises at least the following steps: 1) introducing a double-strand break at a site of interest of the human IL2RG gene comprising at least one recognition and cleavage site of said meganuclease, by contacting said cleavage site with said meganuclease; 2) providing a targeting DNA construct comprising the sequence to be introduced flanked by sequences sharing homologies to the targeted locus. Said meganuclease can be provided directly to the cell or through an expression vector comprising the polynucleotide sequence encoding said meganuclease and suitable for its expression in the used cell. This strategy is used to introduce a DNA sequence at the target site, for example to generate knock-in or transgenic animals, or recombinant human cell lines that can be used for protein production, gene function studies, drug development (drug screening) or as SCID-X1 model (study of the disease and of the correction of the mutations by meganuclease-induced homologous recombination).

In a second embodiment of the use of the meganuclease according to the present invention, it comprises at least the following steps: 1) introducing a double-strand break at a site of interest of the human IL2RG gene comprising at least one recognition and cleavage site of said meganuclease, by contacting said cleavage site with said meganuclease; 2) maintaining said broken genomic locus under conditions appropriate for homologous recombination with chromosomal DNA sharing homologies to regions surrounding the cleavage site.

In a third embodiment of the use of the meganuclease according to the present invention, it comprises at least the following steps: 1) introducing a double-strand break at a site of interest of the human IL2RG gene comprising at least one recognition and cleavage site of said meganuclease, by contacting said cleavage site with said meganuclease; 2) maintaining said broken genomic locus under conditions appropriate for repair of the double-strand break by non-homologous end joining.

The subject-matter of the present invention is also a method for making a modified mouse (knock-in mouse) derived from a humanized mouse comprising a normal/mutated human IL2RG gene, comprising at least the steps of:

(a) introducing into a pluripotent precursor cell or an embryo of said humanized mouse, a meganuclease, as defined above, so as to induce a double strand cleavage at a site of interest of the human IL2RG gene comprising a DNA recognition and cleavage site of said meganuclease; and simultaneously or consecutively,

(b) introducing into the mouse precursor cell or embryo of step (a) a targeting DNA, wherein said targeting DNA comprises (1) DNA sharing homologies to the region surrounding the cleavage site and (2) DNA which repairs the site of interest upon recombination between the targeting DNA and the chromosomal DNA, so as to generate a genomically modified mouse precursor cell or embryo having repaired the site of interest by homologous recombination,

(c) developping the genomically modified mouse precursor cell or embryo of step (b) into a chimeric mouse, and

(d) deriving a transgenic mouse from the chimeric mouse of step (c).

Preferably, step (c) comprises the introduction of the genomically modified precursor cell generated in step (b) into blastocysts so as to generate chimeric mice.

The subject-matter of the present invention is also a method for making a recombinant human cell, comprising at least the steps of:

(a) introducing into a human cell, a meganuclease, as defined above, so as to induce a double stranded cleavage at a site of interest of the human IL2RG gene comprising a DNA recognition and cleavage site for said meganuclease, and simultaneously or consecutively,

(b) introducing into the cell of step (a), a targeting DNA, wherein said targeting DNA comprises (1) DNA sharing homologies to the region surrounding the cleavage site and (2) DNA which repairs the site of interest upon recombination between the targeting DNA and the chromosomal DNA, so as to generate a recombinant human cell having repaired the site of interest by homologous recombination,

(c) isolating the recombinant human cell of step (b), by any appropriate mean.

The targeting DNA is introduced into the cell under conditions appropriate for introduction of the targeting DNA into the site of interest.

In a preferred embodiment, said targeting DNA construct is inserted in a vector.

The cells which are modified may be any cells of interest. For making knock-in/transgenic mice, the cells are pluripotent precursor cells such as embryo-derived stem (ES) cells, which are well-known in the art. For making recombinant human cell lines, the cells may advantageously be PerC6 (Fallaux et al., Hum. Gene Ther. 9, 1909-1917, 1998) or HEK293 (ATCC #CRL-1573) cells. Said meganuclease can be provided directly to the cell or through an expression vector comprising the polynucleotide sequence encoding said meganuclease and suitable for its expression in the used cell.

For making human recombinant cell lines/transgenic animals expressing an heterologous protein of interest, the targeting DNA comprises a sequence encoding the product of interest (protein or RNA), and eventually a marker gene, flanked by sequences upstream and downstream the cleavage site, as defined above, so as to generate genomically modified cells (human cell) having integrated the exogenous sequence of interest in the human IL2RG gene, by homologous recombination.

The sequence of interest may be any gene coding for a certain protein/peptide of interest, including but not limited to: reporter genes, receptors, signaling molecules, transcription factors, pharmaceutically active proteins and peptides, disease causing gene products and toxins. The sequence may also encode an RNA molecule of interest including for example a siRNA.

The expression of the exogenous sequence may be driven, either by the endogenous human IL2RG promoter or by an heterologous promoter, preferably a ubiquitous or tissue specific promoter, either constitutive or inducible, as defined above. In addition, the expression of the sequence of interest may be conditional; the expression may be induced by a site-specific recombinase (Cre, FLP . . . ).

Thus, the sequence of interest is inserted in an appropriate cassette that may comprise an heterologous promoter operatively linked to said gene of interest and one or more functional sequences including but not limited to (selectable) marker genes, recombinase recognition sites, polyadenylation signals, splice acceptor sequences, introns, tags for protein detection and enhancers.

For making animal models of SCID-X1, the targeting DNA comprises the correct/mutated human IL2RG gene sequence, flanked by sequences upstream and downstream the cleavage site, so as to generate animals having corrected the mutation in the IL2RG gene or animals having inserted a mutated IL2RG gene that causes SCID-X1 in human, so as to study gene correction by meganuclease-induced homologous recombination.

The meganuclease can be used either as a polypeptide or as a polynucleotide construct encoding said polypeptide. It is introduced into mouse cells, by any convenient means well-known to those in the art, which are appropriate for the particular cell type, alone or in association with either at least an appropriate vehicle or carrier and/or with the targeting DNA.

According to an advantageous embodiment of the uses according to the invention, the meganuclease (polypeptide) is associated with:

liposomes, polyethyleneimine (PEI); in such a case said association is administered and therefore introduced into somatic target cells.

membrane translocating peptides (Bonetta, The Scientist, 2002, 16, 38; Ford et al., Gene Ther., 2001, 8, 1-4; Wadia and Dowdy, Curr. Opin. Biotechnol., 2002, 13, 52-56); in such a case, the sequence of the variant/single-chain meganuclease is fused with the sequence of a membrane translocating peptide (fusion protein).

According to another advantageous embodiment of the uses according to the invention, the meganuclease (polynucleotide encoding said meganuclease) and/or the targeting DNA is inserted in a vector. Vectors comprising targeting DNA and/or nucleic acid encoding a meganuclease can be introduced into a cell by a variety of methods (e.g., injection, direct uptake, projectile bombardment, liposomes, electroporation). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

Once in a cell, the meganuclease and if present, the vector comprising targeting DNA and/or nucleic acid encoding a meganuclease are imported or translocated by the cell from the cytoplasm to the site of action in the nucleus.

For purposes of therapy, the meganucleases and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results in a decrease in the severity of one or more symptoms of the targeted disease and in a genome correction of the lesion or abnormality.

In one embodiment of the uses according to the present invention, the meganuclease is substantially non-immunogenic, i.e., engender little or no adverse immunological response. A variety of methods for ameliorating or eliminating deleterious immunological reactions of this sort can be used in accordance with the invention. In a preferred embodiment, the meganuclease is substantially free of N-formyl methionine. Another way to avoid unwanted immunological reactions is to conjugate meganucleases to polyethylene glycol (“PEG”) or polypropylene glycol (“PPG”) (preferably of 500 to 20,000 daltons average molecular weight (MW)). Conjugation with PEG or PPG, as described by Davis et al. (U.S. Pat. No. 4,179,337) for example, can provide non-immunogenic, physiologically active, water soluble endonuclease conjugates with anti-viral activity. Similar methods also using a polyethylene—polypropylene glycol copolymer are described in Saifer et al. (U.S. Pat. No. 5,006,333).

The invention also concerns a prokaryotic or eukaryotic host cell which is modified by a polynucleotide or a vector as defined above, preferably an expression vector.

The invention also concerns a non-human transgenic animal or a transgenic plant, characterized in that all or part of their cells are modified by a polynucleotide or a vector as defined above.

As used herein, a cell refers to a prokaryotic cell, such as a bacterial cell, or an eukaryotic cell, such as an animal, plant or yeast cell.

The subject-matter of the present invention is also the use of at least one meganuclease variant, as defined above, as a scaffold for making other meganucleases. For example, further rounds of mutagenesis and selection/screening can be performed on said variants, for the purpose of making novel meganucleases.

The different uses of the meganuclease and the methods of using said meganuclease according to the present invention include the use of the I-CreI variant, the single-chain chimeric meganuclease derived from said variant, the polynucleotide(s), vector, cell, transgenic plant or non-human transgenic mammal encoding said variant or single-chain chimeric meganuclease, as defined above.

The I-CreI variant according to the invention may be obtained by a (global combinatorial) method for engineering I-CreI variants able to cleave a genomic DNA target sequence from the human IL2RG gene, comprising at least the steps of:

(a) constructing a first series of I-CreI variants having at least one substitution in a first functional subdomain of the LAGLIDADG core domain (SEQ ID NO: 166) situated from positions 26 to 40 of I-CreI,

(b) constructing a second series of I-CreI variants having at least one substitution in a second functional subdomain of the LAGLIDADG core domain (SEQ ID NO: 166) situated from positions 44 to 77 of I-CreI,

(c) selecting and/or screening the variants from the first series of step (a) which are able to cleave a mutant I-CreI site wherein (i) the nucleotide triplet at positions −10 to −8 of the I-CreI site has been replaced with the nucleotide triplet which is present at positions −10 to −8 of said genomic target and (ii) the nucleotide triplet at positions +8 to +10 has been replaced with the reverse complementary sequence of the nucleotide triplet which is present at positions −10 to −8 of said genomic target,

(d) selecting and/or screening the variants from the second series of step (b) which are able to cleave a mutant I-CreI site wherein (i) the nucleotide triplet at positions −5 to −3 of the I-CreI site has been replaced with the nucleotide triplet which is present at positions −5 to −3 of said genomic target and (ii) the nucleotide triplet at positions +3 to +5 has been replaced with the reverse complementary sequence of the nucleotide triplet which is present at positions −5 to −3 of said genomic target,

(e) selecting and/or screening the variants from the first series of step (a) which are able to cleave a mutant I-CreI site wherein (i) the nucleotide triplet at positions +8 to +10 of the I-CreI site has been replaced with the nucleotide triplet which is present at positions +8 to +10 of said genomic target and (ii) the nucleotide triplet at positions −10 to −8 has been replaced with the reverse complementary sequence of the nucleotide triplet which is present at positions +8 to +10 of said genomic target,

(f) selecting and/or screening the variants from the second series of step (b) which are able to cleave a mutant I-CreI site wherein (i) the nucleotide triplet at positions +3 to +5 of the I-CreI site has been replaced with the nucleotide triplet which is present at positions +3 to +5 of said genomic target and (ii) the nucleotide triplet at positions −5 to −3 has been replaced with the reverse complementary sequence of the nucleotide triplet which is present at positions +3 to +5 of said genomic target,

(g) combining in a single variant, the mutation(s) at positions 26 to 40 and 44 to 77 of two variants from step (c) and step (d), to obtain a novel homodimeric I-CreI variant which cleaves a sequence wherein (i) the nucleotide triplet at positions −10 to −8 is identical to the nucleotide triplet which is present at positions −10 to −8 of said genomic target, (ii) the nucleotide triplet at positions +8 to +10 is identical to the reverse complementary sequence of the nucleotide triplet which is present at positions −10 to −8 of said genomic target, (iii) the nucleotide triplet at positions −5 to −3 is identical to the nucleotide triplet which is present at positions −5 to −3 of said genomic target and (iv) the nucleotide triplet at positions +3 to +5 is identical to the reverse complementary sequence of the nucleotide triplet which is present at positions −5 to −3 of said genomic target, and/or

(h) combining in a single variant, the mutation(s) at positions 26 to 40 and 44 to 77 of two variants from step (e) and step (f), to obtain a novel homodimeric I-CreI variant which cleaves a sequence wherein (i) the nucleotide triplet at positions +3 to +5 is identical to the nucleotide triplet which is present at positions +3 to +5 of said genomic target, (ii) the nucleotide triplet at positions −5 to −3 is identical to the reverse complementary sequence of the nucleotide triplet which is present at positions +3 to +5 of said genomic target, (iii) the nucleotide triplet at positions +8 to +10 of the I-CreI site has been replaced with the nucleotide triplet which is present at positions +8 to +10 of said genomic target and (iv) the nucleotide triplet at positions −10 to −8 is identical to the reverse complementary sequence of the nucleotide triplet at positions +8 to +10 of said genomic target,

(i) combining the variants obtained in steps (g) and (h) to form heterodimers, and

(j) selecting and/or screening the heterodimers from step (i) which are able to cleave said genomic DNA target from the human IL2RG gene.

One of the step(s) (c), (d), (e) or (f) may be omitted. For example, if step (c) is omitted, step (d) is performed with a mutant I-CreI site wherein both nucleotide triplets at positions −10 to −8 and −5 to −3 have been replaced with the nucleotide triplets which are present at positions −10 to −8 and −5 to −3, respectively of said genomic target, and the nucleotide triplets at positions +3 to +5 and +8 to +10 have been replaced with the reverse complementary sequence of the nucleotide triplets which are present at positions −5 to −3 and −10 to −8, respectively of said genomic target.

The (intramolecular) combination of mutations in steps (g) and (h) may be performed by amplifying overlapping fragments comprising each of the two subdomains, according to well-known overlapping PCR techniques.

The (intermolecular) combination of the variants in step (i) is performed by co-expressing one variant from step (g) with one variant from step (h), so as to allow the formation of heterodimers. For example, host cells may be modified by one or two recombinant expression vector(s) encoding said variant(s). The cells are then cultured under conditions allowing the expression of the variant(s), so that heterodimers are formed in the host cells, as described previously in the International PCT Application WO 2006/097854 and Arnould et al., J. Mol. Biol., 2006, 355, 443-458.

The selection and/or screening in steps (c), (d), (e), (f) and/or (j) may be performed by using a cleavage assay in vitro or in vivo, as described in the International PCT Application WO 2004/067736, Arnould et al., J. Mol. Biol., 2006, 355, 443-458, Epinat et al., Nucleic Acids Res., 2003, 31, 2952-2962 and Chames et al., Nucleic Acids Res., 2005, 33, e178.

According to another advantageous embodiment of said method, steps (c), (d), (e), (f) and/or (j) are performed in vivo, under conditions where the double-strand break in the mutated DNA target sequence which is generated by said variant leads to the activation of a positive selection marker or a reporter gene, or the inactivation of a negative selection marker or a reporter gene, by recombination-mediated repair of said DNA double-strand break.

Steps (a), (b), (g), (h) and (i) may further comprise the introduction of additional mutations at other positions contacting the DNA target sequence or interacting directly or indirectly with said DNA target, at positions which improve the binding and/or cleavage properties of the mutants, or at positions which either prevent or impair the formation of functional homodimers or favor the formation of the heterodimer, as defined above.

The additional mutations may be introduced by site-directed mutagenesis and/or random mutagenesis on a variant or on a pool of variants, according to standard mutagenesis methods which are well-known in the art, for example by using PCR. Site-directed mutagenesis may be advantageously performed by amplifying overlapping fragments comprising the mutated position(s), as defined above, according to well-known overlapping PCR techniques. In addition, multiple site-directed mutagenesis, may advantageously be performed on a variant or on a pool of variants.

In particular, random mutations may be introduced on the whole variant or in a part of the variant, in particular the C-terminal half of the variant (positions 80 to 163) to improve the binding and/or cleavage properties of the mutants towards the DNA target from the gene of interest. Site-directed mutagenesis at positions which improve the binding and/or cleavage properties of the mutants, for example at positions 19, 54, 80, 87, 105 and /or 132, may also be combined with random-mutagenesis. The mutagenesis may be performed by generating random/site-directed mutagenesis libraries on a pool of variants, according to standard mutagenesis methods which are well-known in the art.

Preferably, the mutagenesis is performed on one monomer of the heterodimer formed in step (i) or obtained in step (j), advantageously on a pool of monomers, preferably on both monomers of the heterodimer of step (i) or (j).

Preferably, at least two rounds of selection/screening are performed according to the process illustrated by FIG. 4 of Arnould et al., J. Mol. Biol., 2007, 371, 49-65. In the first round, one of the monomers of the heterodimer is mutagenised (monomer Y in FIG. 4), co-expressed with the other monomer (monomer X in FIG. 4) to form heterodimers, and the improved monomers Y⁺ are selected against the target from the gene of interest. In the second round, the other monomer (monomer X) is mutagenised, co-expressed with the improved monomers Y⁺ to form heterodimers, and selected against the target from the gene of interest to obtain meganucleases (X⁺) Y⁺) with improved activity. The mutagenesis may be random-mutagenesis or site-directed mutagenesis on a monomer or on a pool of monomers, as indicated above. Both types of mutagenesis are advantageously combined. Additional rounds of selection/screening on one or both monomers may be performed to improve the cleavage activity of the variant.

The cleavage activity of the improved meganuclease obtainable by the method according to the present invention may be measured by a direct repeat recombination assay, in yeast or mammalian cells, using a reporter vector, by comparison with that of the initial meganuclease. The reporter vector comprises two truncated, non-functional copies of a reporter gene (direct repeats) and the genomic DNA target sequence which is cleaved by the initial meganuclease, within the intervening sequence, cloned in a yeast or a mammalian expression vector. Expression of the meganuclease results in cleavage of the genomic DNA target sequence. This cleavage induces homologous recombination between the direct repeats, resulting in a functional reporter gene (LacZ, for example), whose expression can be monitored by appropriate assay. A stronger signal is observed with the improved meganuclease, as compared to the initial meganuclease. Alternatively, the activity of the improved meganuclease towards its genomic DNA target can be compared to that of I-CreI towards the I-CreI site, at the same genomic locus, using a chromosomal assay in mammalian cells (Arnould et al., J. Mol. Biol., 2007, 371, 49-65).

Furthermore, the homodimeric combined variants obtained in step (g) or (h) are advantageously submitted to a selection/screening step to identify those which are able to cleave a pseudo-palindromic sequence wherein at least the nucleotides at positions −11 to −3 (combined variant of step (g)) or +3 to +11 (combined variant of step (h)) are identical to the nucleotides which are present at positions −11 to −3 (combined variant of step (g)) or +3 to +11 (combined variant of step (h)) of said genomic target, and the nucleotides at positions +3 to +11 (combined variant of step (g)) or −11 to −3 (combined variant of step (h)) are identical to the reverse complementary sequence of the nucleotides which are present at positions −11 to −3 (combined variant of step (g)) or +3 to +11 (combined variant of step (h)) of said genomic target.

Preferably, the set of combined variants of step (g) or step (h) (or both sets) undergoes an additional selection/screening step to identify the variants which are able to cleave a pseudo-palindromic sequence wherein : (i) the nucleotides at positions −2 to +2 (four central bases) are identical to the nucleotides which are present at positions −2 to +2 of said genomic target, (ii) the nucleotides at positions −11 to −3 (combined variant of step g)) or +3 to +11 (combined variant of step (h)) are identical to the nucleotides which are present at positions −11 to −3 (combined variant of step (g)) or +3 to +11 (combined variant of step h)) of said genomic target, and (iii) the nucleotides at positions +3 to +11 (combined variant of step (g)) or −11 to −3 (combined variant of step (h)) are identical to the reverse complementary sequence of the nucleotides which are present at positions −11 to −3 (combined variant of step (g)) or +3 to +11 (combined variant of step (h)) of said genomic target. This additional screening step increases the probability of isolating heterodimers which are able to cleave the genomic target of interest (step (j)).

Alternatively, the I-CreI variant according to the invention may be obtained by a sequential combinatorial method for engineering I-CreI variants able to cleave a DNA target sequence from a genome of interest (from a eukaryote such as a mammal (human) or a plant or from a microorganism such as a virus), comprising at least the steps of:

(a₁) constructing a first series of I-CreI variants having at least one substitution in a first functional subdomain of the LAGLIDADG core domain (SEQ ID NO: 166) situated from positions 44 to 77 of I-CreI, preferably at positions 44, 68, 70, 75 and/or 77,

(b₁) selecting and/or screening the variants from the first series of step (a₁) which are able to cleave a mutant I-CreI site wherein at least the nucleotides at positions +3 to +5 of the I-CreI site have been replaced with the nucleotides which are present at positions +3 to +5 of said genomic target and the nucleotides at positions −5 to −3 have been replaced with the reverse complementary sequence of the nucleotides which are present at positions +3 to +5 of said genomic target, preferably the nucleotides at positions +3 to +7 and +11 of the I-CreI site have been replaced with the nucleotides which are present at positions +3 to +7 and +11 of said genomic target and the nucleotides at positions −11, and −7 to −3 have been replaced with the reverse complementary sequence of the nucleotides which are present at positions +3 to +7 and +11 of said genomic target,

(c₁) constructing a second series of I-CreI variants from the variants obtained in step (b₁), said variants having at least one substitution in a second functional subdomain of the LAGLIDADG core domain (SEQ ID NO: 166) situated from positions 26 to 40 of I-CreI, preferably at positions 28, 30, 32, 33, 38 and/or 40,

(d₁) selecting and/or screening the variants from step (c₁) which are able to cleave a mutant I-CreI site wherein at least the nucleotides at positions +3 to +5 and +8 to +10 of the I-CreI site have been replaced with the nucleotides which are present at positions +3 to +5 and +8 to +10 of said genomic target and the nucleotides at positions −10 to −8 and −5 to −3 have been replaced with the reverse complementary sequence of the nucleotides which are present at positions +3 to +5 and +8 to +10 of said genomic target, preferably the nucleotides at positions +3 to +11 of the I-CreI site have been replaced with the nucleotides which are present at positions +3 to +11 of said genomic target and the nucleotides at positions −11 to −3 have been replaced with the reverse complementary sequence of the nucleotides which are present at positions +3 to +11 of said genomic target,

(e₁) combining the variants obtained in step (d₁) with I-CreI variants having mutations at positions 26 to 40 and/or 44 to 77 which are able to cleave a mutant I-CreI site wherein at least the nucleotides at positions −10 to −8 and −5 to −3 of the I-CreI site have been replaced with the nucleotides which are present at positions −10 to −8 and −5 to −3 of said genomic target and at least the nucleotides at positions +3 to +5 and +8 to +10 have been replaced with the reverse complementary sequence of the nucleotides which are present at positions −10 to −8 and −5 to −3 of said genomic target, to form heterodimers; preferably, the I-CreI variants having mutations at positions 26 to 40 and/or 44 to 77 are able to cleave a mutant I-CreI site wherein the nucleotides at positions −11 to −3 of the I-CreI site have been replaced with the nucleotides which are present at positions −11 to −3 of said genomic target and the nucleotides at positions +3 to +11 have been replaced with the reverse complementary sequence of the nucleotides which are present at positions −11 to −3 of said genomic target, and

(f₁) selecting and/or screening the heterodimers from step (e₁) which are able to cleave said genomic DNA target of interest.

Alternatively, step (a₁) to (c₁) of the sequential combinatorial method may be replaced by steps (a′₁) to (c′₁):

(a′₁) constructing a first series of I-CreI variants having at least one substitution in the functional subdomain of the LAGLIDADG core domain (SEQ ID NO: 166) situated from positions 26 to 40 of I-CreI, preferably at positions 28, 30, 32, 33, 38 and/or 40,

(b′₁) selecting and/or screening the variants from the first series of step (a₁) which are able to cleave a mutant I-CreI site wherein at least the nucleotides at positions +8 to +10 of the I-CreI site have been replaced with the nucleotides which are present at positions +8 to +10 of said genomic target and the nucleotides at positions −10 to −8 have been replaced with the reverse complementary sequence of the nucleotides which are present at positions +8 to +10 of said genomic target, preferably, the nucleotides at positions +6 to +11 of the I-CreI site have been replaced with the nucleotides which are present at positions +6 to +11 of said genomic target and the nucleotides at positions −11 to −6 have been replaced with the reverse complementary sequence of the nucleotides which are present at positions +6 to +11 of said genomic target,

(c′₁) constructing a second series of I-CreI variants from the variants obtained in step (b′₁), said variants having at least one substitution in the functional subdomain of the LAGLIDADG core domain (SEQ ID NO: 166) situated from positions 44 to 77 of I-CreI, preferably at positions 44, 68, 70, 75 and/or 77.

The variants obtained in step (d₁) form one of the two monomers (the first monomer) of the heterodimers obtained in step (f₁). To engineer variants forming the other monomer (second monomer) of the heterodimers obtained in step (f₁), the sequential combinatorial method comprises:

the steps (a₁) or (a′₁), (c₁) or (c′₁) and (f₁), as defined above,

steps (b₁) or (b′₁) and (d₁), wherein the mutant I-CreI site has at least nucleotides at positions −5 to −3 (step b₁), −10 to −8 (step b′₁) or −10 to −8 and −5 to −3 (step d₁) which have been replaced with the nucleotides which are present at positions −5 to −3 (step b₁), −10 to −8 (step b′₁) or −10 to −8 and −5 to −3 (step d₁) of the genomic target and at least the nucleotides at positions +3 to +5 (step b₁), +8 to +10 (step b′₁), or +3 to +5 and +8 to +10 (step d₁) have been replaced with the reverse complementary sequence of the nucleotides which are present at positions −5 to −3 (step b₁), −10 to −8 (step b′₁), or −10 to −8 and −5 to −3 (step d₁) of said genomic target, preferably, the mutant I-CreI site has nucleotides at positions −11 and −7 to −3 (step b₁), −11 to −6 (step b′₁), or −11 to −3 (step d₁) which have been replaced with the nucleotides which are present at positions −11 and −7 to −3 (step b₁), −11 to −6 (step b′₁) or −11 to −3 (step d₁) of the genomic target and the nucleotides at positions +3 to +7 and +11 (step b₁), +6 to +11 (step b′₁) or +3 to +11 (step d₁) have been replaced with the reverse complementary sequence of the nucleotides which are present at positions −11 and −7 to −3 (step b₁), −11 to −6 (step b′₁), or −11 to −3 (step d₁) of said genomic target.

a step (c₁) wherein heterodimers are formed by combining the variants obtained in step (d₁) with I-CreI variants forming the other monomer, i.e. I-CreI variants having mutations at positions 26 to 40 and/or 44 to 77 which are able to cleave a mutant I-CreI site wherein at least the nucleotides at positions +3 to +5 and +8 to +10 of the I-CreI site have been replaced with the nucleotides which are present at positions +3 to +5 and +8 to +10 of said genomic target and at least the nucleotides at positions −10 to −8 and −5 to −3 have been replaced with the reverse complementary sequence of the nucleotides which are present at positions +3 to +5 and +8 to +10 of said genomic target; preferably the I-CreI variants forming the other monomer are able to cleave a mutant I-CreI site wherein the nucleotides at positions +3 to +11 of the I-CreI site have been replaced with the nucleotides which are present at positions +3 to +11 of said genomic target and the nucleotides at positions −11 to −3 have been replaced with the reverse complementary sequence of the nucleotides which are present at positions +3 to +11 of said genomic target.

Preferably, the variants obtained in step (d₁) undergo an additional selection/screening step to identify those which are able to cleave a pseudo-palindromic sequence wherein : (i) the nucleotides at positions −2 to +2 (four central bases) are identical to the nucleotides which are present at positions −2 to +2 of said genomic target, (ii) the nucleotides at positions −11 to −3 or +3 to +11 are identical to the nucleotides which are present at positions −11 to −3 or +3 to +11 of said genomic target, and (iii) the nucleotides at positions +3 to +11 or −11 to −3 are identical to the reverse complementary sequence of the nucleotides which are present at positions −11 to −3 or +3 to +11 of said genomic target. This additional screening step increases the probability of isolating heterodimers which are able to cleave the genomic target of interest (step (f₁)).

The series of I-CreI variants in steps (a₁), (a′₁), (c₁), (c′₁) are generated by constructing combinatorial libraries having amino acid variation at positions 28, 30, 32, 33, 38 and/or 40 (first subdomain) or at positions 44, 68, 70, 75 and/or 77 (second subdomain), as described previously in International PCT Applications WO 2004/067736, WO 2006/097784, WO 2006/097853 WO 2007/060495 and WO 2007/049156 ; Arnould et al., J. Mol. Biol., 2006, 355, 443-458; Smith et al., Nucleic Acids Res., 2006, 34, e149.

The selection and/or screening in steps (b₁), (b′₁), (d₁), and/or additional step before step (e₁) may be performed by using a cleavage assay in vitro or in vivo, as described above for the other combinatorial method.

The (intermolecular) combination of the I-CreI variants in step (c₁) is performed by co-expressing the two variants, as described above for the other combinatorial method.

Additional mutations may be introduced in the series of variants of steps (a₁), (a′₁), (c₁), (c′₁) or in the variants obtained in step (b₁), (b′₁) (d₁), additional step before step (e₁) and step (f₁). These mutations may be introduced at other positions contacting the DNA target sequence or interacting directly or indirectly with said DNA target, at positions which improve the binding and/or cleavage properties of the variants, or at positions which either prevent or impair the formation of functional homodimers or favor the formation of the heterodimer, as defined above for the other combinatorial method. Preferably, mutations that improve the binding and/or cleavage properties of the variants are introduced by site-directed or random mutagenesis on the variants obtained in step (d₁) (after the first screening or the the additional screening as described above).

The subject-matter of the present invention is also an I-CreI variant having mutations at positions 26 to 40 and/or 44 to 77 of I-CreI that is useful for engineering the variants able to cleave a DNA target from the human IL2RG gene, according to the present invention. In particular, the invention encompasses the I-CreI variants as defined in step (c) to (f) of the method for engineering I-CreI variants, as defined above, including the variants of Table II and IV. The invention encompasses also the I-CreI variants as defined in step (g) and (h) of the method for engineering I-CreI variants, as defined above, including the variants of the sequence SEQ ID NO: 40, 45, 48 to 111, 115, 120 to 148 and 156 to 162 (combined variants of Tables II, III, V, VII, VIII, IX, XI, XIII, XIV, XV, XVI and XVII).

Single-chain chimeric meganucleases able to cleave a DNA target from the gene of interest are derived from the variants according to the invention by methods well-known in the art (Epinat et al., Nucleic Acids Res., 2003, 31, 2952-62; Chevalier et al., Mol. Cell., 2002, 10, 895-905; Steuer et al., Chembiochem., 2004, 5, 206-13; International PCT Applications WO 03/078619 and WO 2004/031346). Any of such methods, may be applied for constructing single-chain chimeric meganucleases derived from the variants as defined in the present invention.

The polynucleotide sequence(s) encoding the variant as defined in the present invention may be prepared by any method known by the man skilled in the art. For example, they are amplified from a cDNA template, by polymerase chain reaction with specific primers. Preferably the codons of said cDNA are chosen to favour the expression of said protein in the desired expression system.

The recombinant vector comprising said polynucleotides may be obtained and introduced in a host cell by the well-known recombinant DNA and genetic engineering techniques.

The I-CreI variant or single-chain derivative as defined in the present the invention are produced by expressing the polypeptide(s) as defined above; preferably said polypeptide(s) are expressed or co-expressed (in the case of the variant only) in a host cell or a transgenic animal/plant modified by one expression vector or two expression vectors (in the case of the variant only), under conditions suitable for the expression or co-expression of the polypeptide(s), and the variant or single-chain derivative is recovered from the host cell culture or from the transgenic animal/plant.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

In addition to the preceding features, the invention further comprises other features which will emerge from the description which follows, which refers to examples illustrating the I-CreI meganuclease variants and their uses according to the invention, as well as to the appended drawings in which:

FIG. 1 illustrates two different strategies for restoring a functional gene with meganuclease-induced recombination. A. Gene correction. A mutation occurs within a known gene. Upon cleavage by a meganuclease and recombination with a repair matrix the deleterious mutation is corrected. B. Exonic sequences knockin. A mutation occurs within a known gene. The mutated mRNA transcript is featured below the gene. In the repair matrix, exons located downstream of the cleavage site are fused in frame (as in a cDNA), with a polyadenylation site to stop transcription in 3′. Introns and exons sequences can be used as homologous regions. Exonic sequences knock-in results into an engineered gene, transcribed into a mRNA able to code for a functional protein.

FIG. 2 illustrates the modular structure of homing endonucleases and the combinatorial approach for custom meganucleases design: A. Tridimensional structure of the I-CreI homing endonuclease bound to its DNA target. The catalytic core is surrounded by two αββαββ folds forming a saddle-shaped interaction interface above the DNA major groove. B. Different binding sequences derived from the I-CreI target sequence (top right and bottom left) to obtain heterodimers or single chain fusion molecules cleaving non palindromic chimeric targets (bottom right).
C. The identification of smaller independent subunit, i.e., subunit within a single monomer or αββαββfold (top right and bottom left) allows for the design of novel chimeric molecules (bottom right), by combination of mutations within a same monomer. Such molecules are able to cleave palindromic chimeric targets (bottom right).
D. The combination of the two former steps allows a larger combinatorial approach, involving four different subdomains. A large collection of I-CreI derivatives with locally altered specificity is generated. In a first step, couples of novel meganucleases are combined in new homodimeric proteins (by combinations of mutations within a same monomer; “half-meganucleases”) cleaving palindromic targets derived from the target one wants to cleave. Then, the combination of such “half-meganuclease” can result in a heterodimeric species cleaving the target of interest (custom meganuclease). Thus, the identification of a small number of new cleavers for each subdomain allows for the design of a very large number of novel endonucleases with fully redesigned specificity.

FIG. 3 represents the human IL2RG gene (Accession number NC_{—}000023; SEQ ID NO: 3). Exons sequences are boxed, and their junctions are indicated. ORF is indicated as a grey box. The IL2RG3 target sequence as well as other potential meganuclease sites (IL2RGn) are indicated with their sequences and positions.

FIG. 4 represents the IL2RG3 target sequences and its derivatives. All targets are aligned with the C1221 target (SEQ ID NO: 2), a palindromic sequence cleaved by I-CreI. 10GAC_P, 10GAA_P, 5CTG_P and 5AGG_P (SEQ ID NO: 10 to 15, 114) are close derivatives found to be cleaved by I-CreI mutants. They differ from C1221 by the boxed motives. IL2C_P (SEQ ID NO: 149)differs from 5AGG_P by the bases at position ±11 and ±7. The IL2RG3.6 target (SEQ ID NO: 150) differs from IL2RG3.4 by the boxed four central bases. C1221, 10GAC_P, 10GAA_P, 5CTG_P and 5AGG_P were first described as 24 by sequences, but structural data suggest that only the 22 by are relevant for protein/DNA interaction. However, positions ±12 are indicated in parenthesis. IL2RG3 (SEQ ID NO: 7) is the DNA sequence located in the human IL2RG gene at position 1686. In the IL2RG3.2 target (SEQ ID NO: 12), the TCTC sequence in the middle of the target is replaced with GTAC, the bases found in C1221. IL2RG3.3 (SEQ ID NO: 13) is the palindromic sequence derived from the left part of IL2RG3.2, and IL2RG3.4 (SEQ ID NO: 14) is the palindromic sequence derived from the right part of IL2RG3.2. As shown in the Figure, the boxed motives from 10GAC_P, 10GAA_P, 5CTG_P and 5AGG_P are found in the IL2RG3 series of targets.

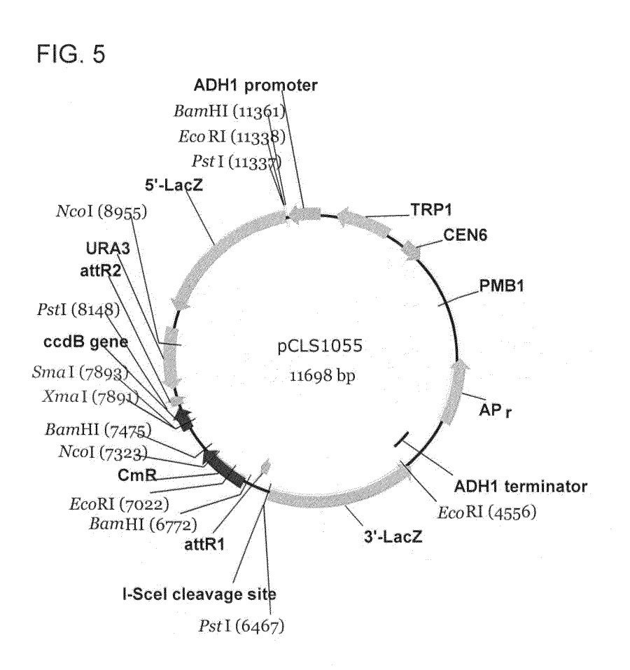

FIG. 5 represents the pCLS1055 plasmid map.

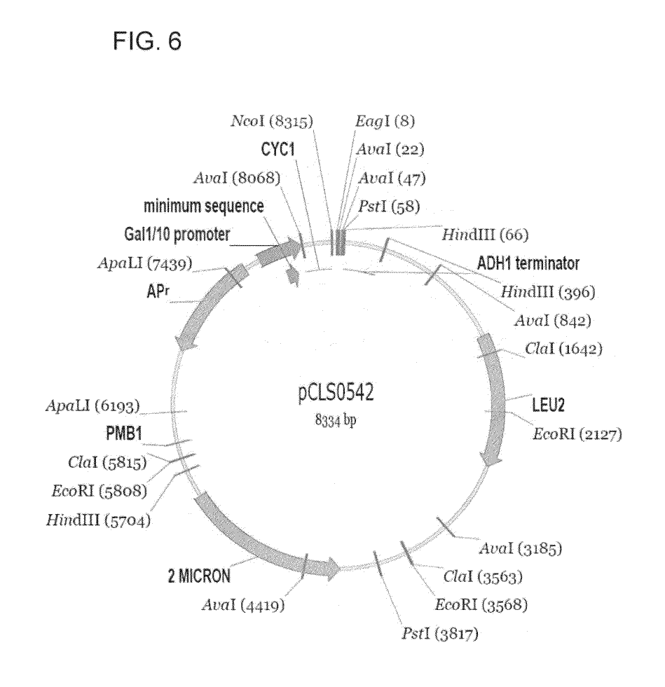

FIG. 6 represents the pCLS0542 plasmid map.

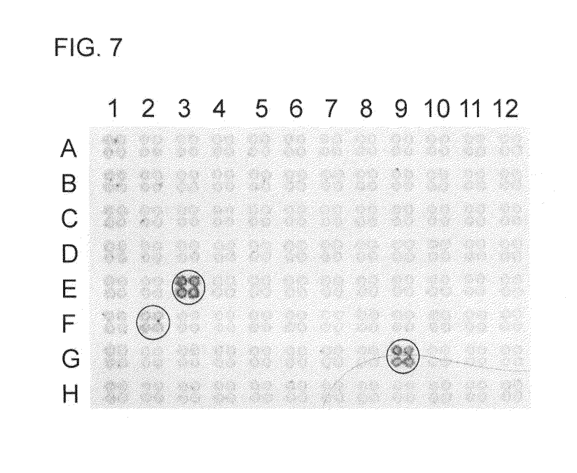

FIG. 7 illustrates cleavage of IL2RG3.3 target by combinatorial mutants. The figure displays an example of primary screening of I-CreI combinatorial mutants with the IL2RG3.3 target. In the filter, the sequences of positive mutants at position E3, F2 and G9 are KHQS/KYSEQ, KRQS/RYSDQ and KHQS/RYSDQ, respectively (according to Tables II and III).

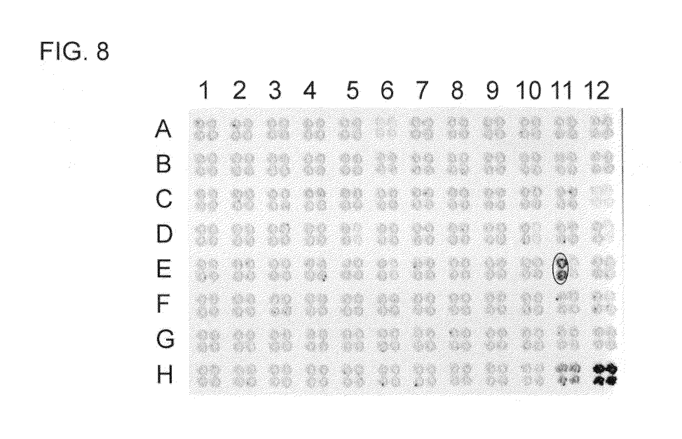

FIG. 8 illustrates cleavage of IL2RG3.4 target by combinatorial mutants. The figure displays an example of primary screening of I-CreI combinatorial mutants with the IL2RG3.4 target. Two 96 well plaques in a 2×2 points screening format. H11 and H12 are positive controls of different strength. In the filter, the sequence of the positive mutant at position E11 is RTYQS/AYSER (according to Table V).

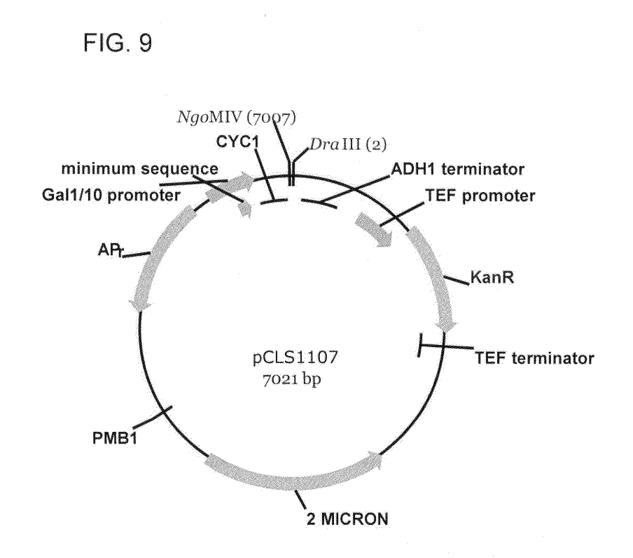

FIG. 9 represents the pCLS1107 plasmid map.

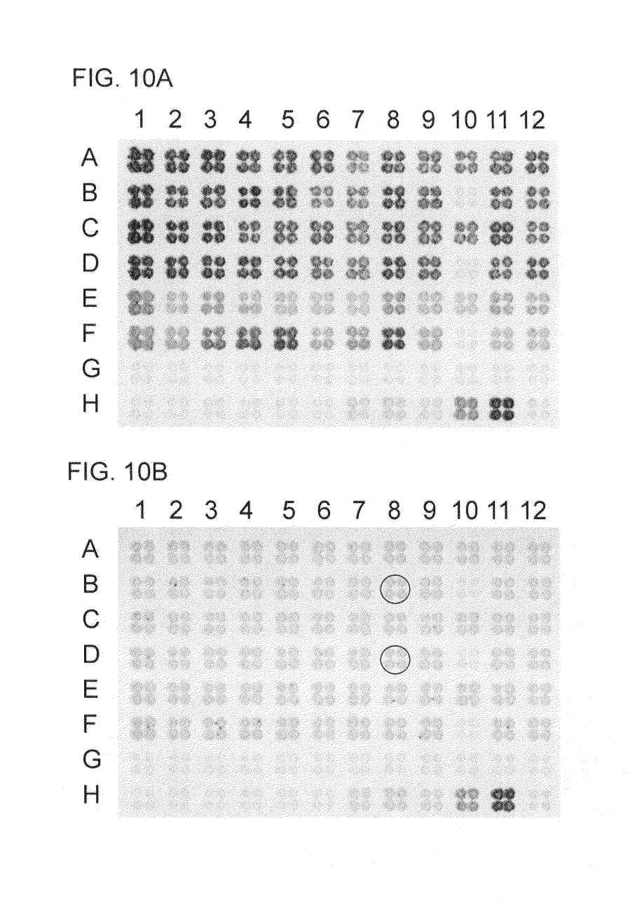

FIG. 10 illustrates cleavage of IL2RG3.2 target sequence by heterodimeric combinatorial mutants. A. Screening of combinations of I-CreI mutants against the IL2RG3.2 target. B. Screening of the same combinations of I-CreI mutants against the IL2RG3 target. A weak signal is observed with this sequence at positions B8 and D8 corresponding to yeast coexpressing mutants m10 and M1 in duplicate. In lanes A, B, C, D: heterodimers are ml to m20 mutants cleaving IL2RG3.3 coexpressed with the M1 mutant cleaving IL2RG3.4. In lanes E and F: heterodimers are m1 to m20 mutants cleaving IL2RG3.3 coexpressed with the M2 mutant cleaving IL2RG3.4. ml to m20 mutants are described in example 2 (Tables II and III). M1 and M2 mutants are described in example 3 (Table V). H10 and H11 are positive controls of different strength.

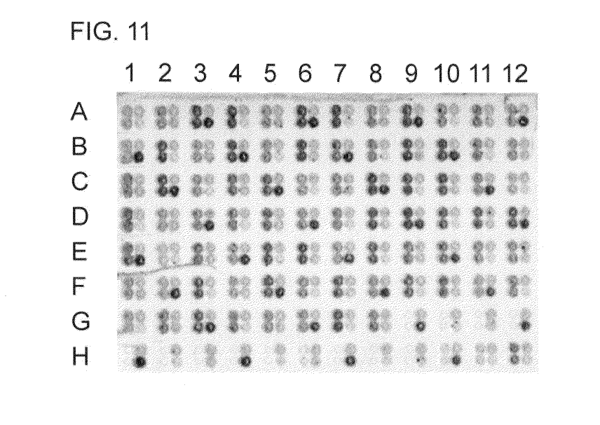

FIG. 11 illustrates cleavage of the IL2RG3 target. Secondary screen example of I-CreI refined mutants obtained by random mutagenesis (example 5) and coexpressed with a mutant cutting IL2RG3.4 (RTYQS/AYSER according to Table V). Cleavage is tested against the IL2RG3 target.

In each cluster: the 2 left spots are the heterodimer in duplicate (except H10, H11 and H12 which are negative and positive controls of different strength); the right spots are controls.

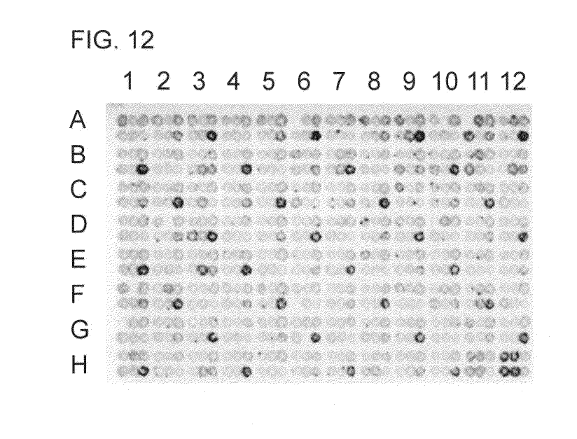

FIG. 12 illustrates cleavage of the IL2RG3 target. Example of primary screen against the IL2RG3 target of the libraries constructed in example 6 by site-directed mutagenesis of initial mutants cleaving the IL2RG3.3 target and optimized mutants derived from them. The figure shows the results obtained for the library containing the G19S substitution. 372 yeast clones are mated with a “mutant-target” yeast strain that (i) contains the IL2RG3 target in a reporter plasmid (ii) expresses the M1 mutant (RTYQS/AYSER according to Table V), a variant cleaving the IL2RG3.4 target described in example 3.

Each cluster contains 6 spots. In the 4 left spots, 4 clones from the library are mated with the “mutant-target” yeast (except for H10, H11 and H12: negative and positive controls of different strength). In the top right spot, a yeast strain expressing one of the 6 mutants described in Table VII in example 5 is mated with the “mutant-target” yeast as a control. And the down right spots are negative and positive controls of different strength.

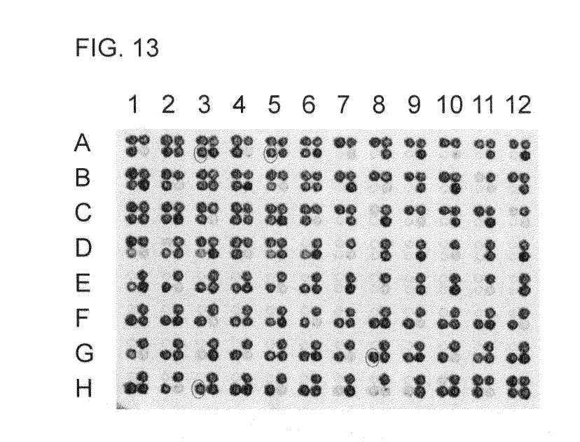

FIG. 13 illustrates cleavage of the IL2RG3 target. Example of screen of optimized mutants derived from the mutant cleaving the IL2RG3.4 target by site-directed mutagenesis described in example 7. In this example, circled spots are:
A3: screen result of the heterodimer formed by .4_R1 and .3_R17 against the IL2RG3 target (according to Table X).
A5: screen result of the heterodimer formed by .4_R2 and .3_R17 against the IL2RG3 target (according to Table X).
G8: screen result of the heterodimer formed by .4_R3 and .3_R17 against the IL2RG3 target (according to Table X).
H3: screen result of the heterodimer formed by .4_R0 and .3_R17 against the IL2RG3 target (according to Table X).

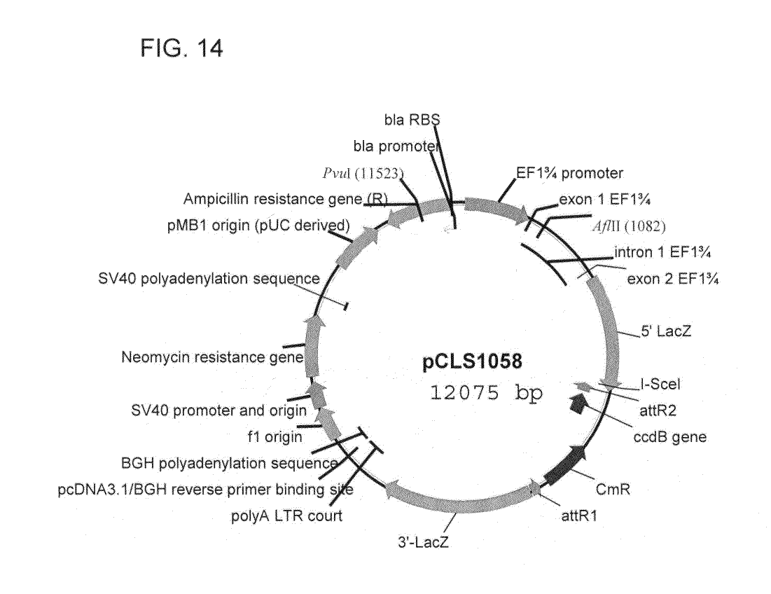

FIG. 14 represents the pCLS1058 plasmid map.

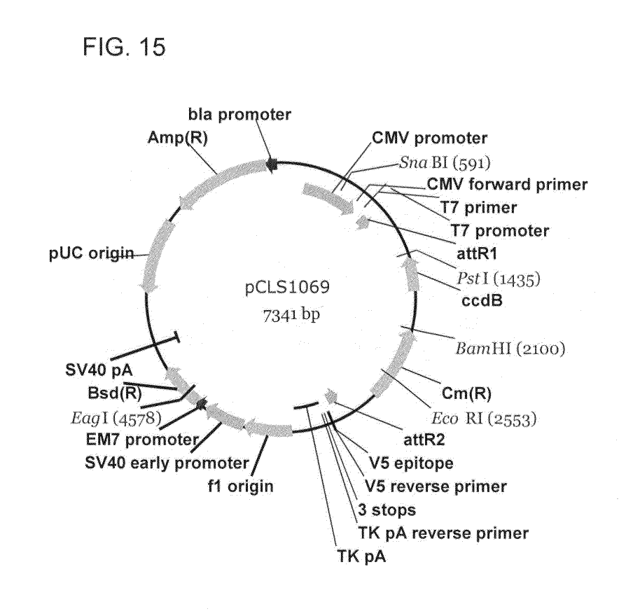

FIG. 15 represents the pCLS1069 plasmid map.

FIG. 16 illustrates refinement of mutant cleaving IL2RG3.4 by random mutagenesis and cleavage of the IL2RG3 target in CHO cells. OD values for the mutants described in example 8 in the CHO assay against the IL2RG3 target. Grey bars consist of the heterodimers where refined mutants are coexpressed with the .3_R17 (26R 31R 33H 44K 54L 68Y 70S 75E 77V 139R I-CreI mutant) and black ones are homodimers containing only the refined mutants. Empty pCLS1069 vector and I-CreI N75 cloned in pCLS1069 are used as negative control.

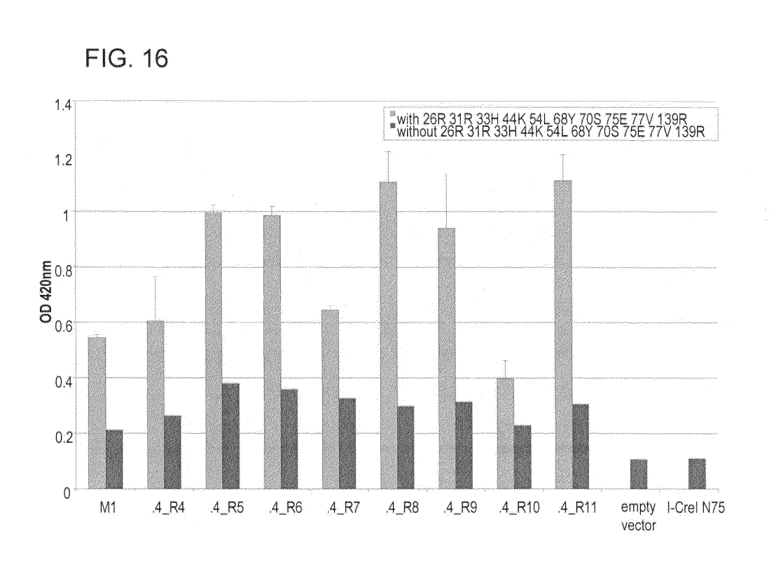

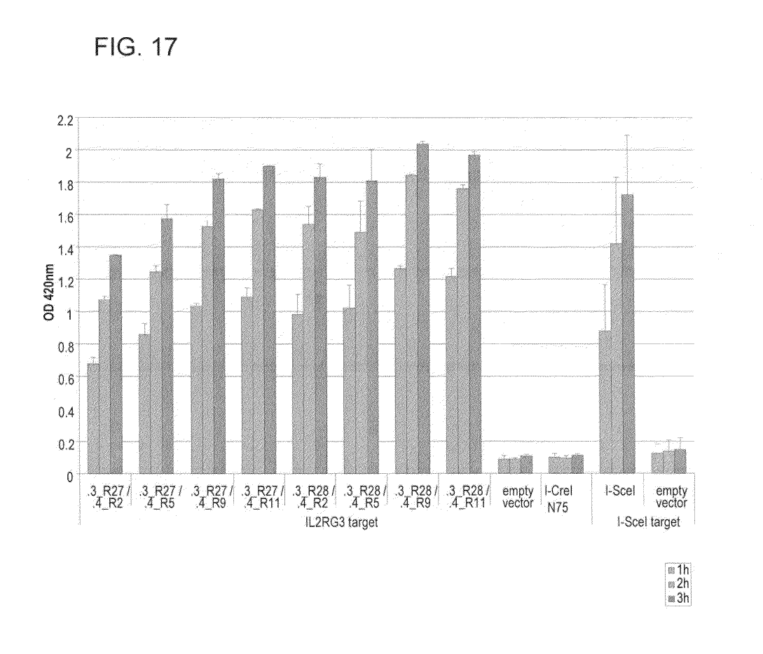

FIG. 17 illustrates IL2RG3 target cleavage in CHO cells. Results of CHO assay for the heterodimers displaying the maximal values against the IL2RG3 target described in example 9. Time course of revelation (OD values are revealed at 3 times: 1 hour (white bars), 2 hours (grey bars) and 3 hours (black bars) after lysis/revelation buffer addition). I-CreI N75 and empty vector are used as negative controls. The I-SceI cleavage of the I-SceI target cloned in pCLS1058 is used as a positive control.

FIG. 18 represents meganuclease target sequences found in the human IL2RG gene and examples of I-CreI variants which are able to cleave said DNA targets; an example of variant (heterodimer formed by a first and a second I-CreI monomer) is presented for each target. The exons closest to the target sequences, and the exons junctions are indicated (columns 1 and 2), the sequence of the DNA target is presented (column 3), with the position of its first nucleotide by reference to SEQ ID NO: 3 (column 4). The minimum repair matrix for repairing the cleavage at the target site is indicated by its first nucleotide (start, column 7) and last nucleotide (end, column 8). The sequence of each I-CreI variant is defined by the mutated residues at the indicated positions. For example, the first heterodimeric variant of FIG. 18 consists of a first monomer having T, Q, N, Y, S, Y and Q at positions 33, 40, 44, 68, 70, 75 and 77, respectively and a second monomer having S, R, K, D, N, S and N at positions 28, 38, 40, 44, 68, 70 and 75, respectively. The positions are indicated by reference to I-CreI sequence SEQ ID NO: 1; I-CreI has K, N, S, Y, Q, S, Q, R, R, D and I, at positions 28, 30, 32, 33, 38, 40, 44, 68, 70, 75 and 77 respectively.

FIG. 19 illustrates some mutations found in SCID-X1 patients.

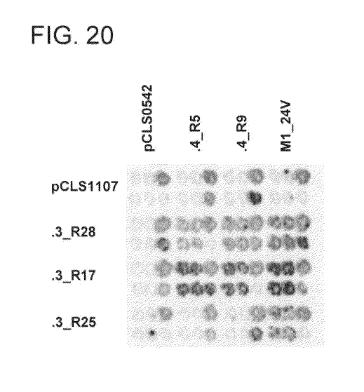

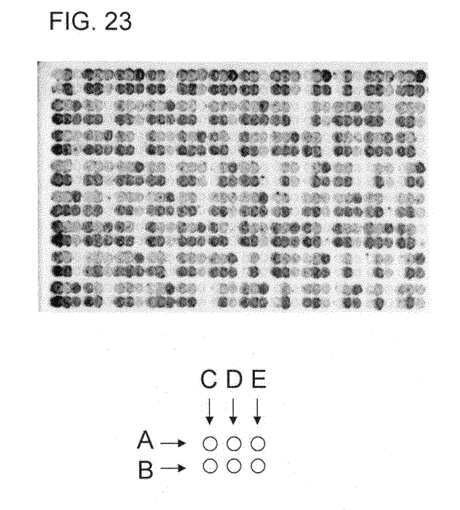

FIG. 20 illustrates cleavage of the IL2RG3 target in yeast. A series of I-CreI optimized mutants derived from M1 mutant cleaving IL2RG3.4 (.4_R5, .4_R9 and M1_{—}24V) are coexpressed in yeast with refined mutants cutting IL2RG3.3 (.3_R17, .3_R25 and .3_R28). Cleavage is tested against the IL2RG3 target. Dark coloration intensity is proportional of cleavage efficiency. In each cluster of 6 spots, the two right points are positive and negative controls, as indicated in the sketch of FIG. 23 (column E).

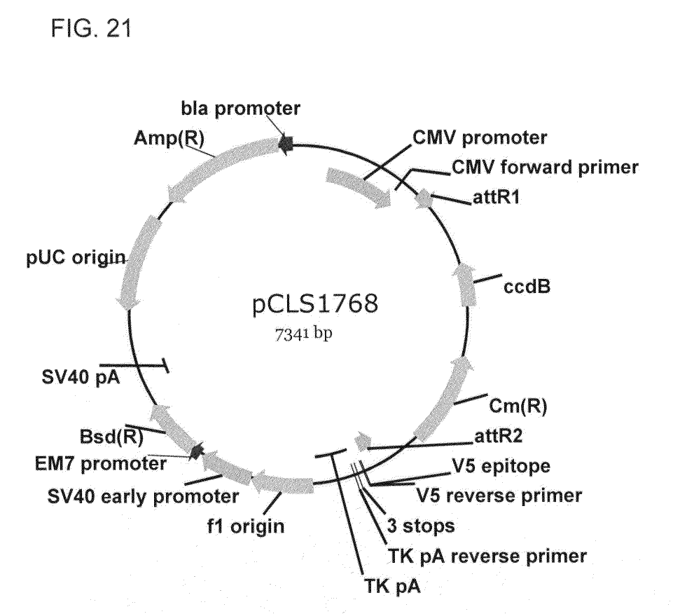

FIG. 21 represents pCLS1768 plasmid map.

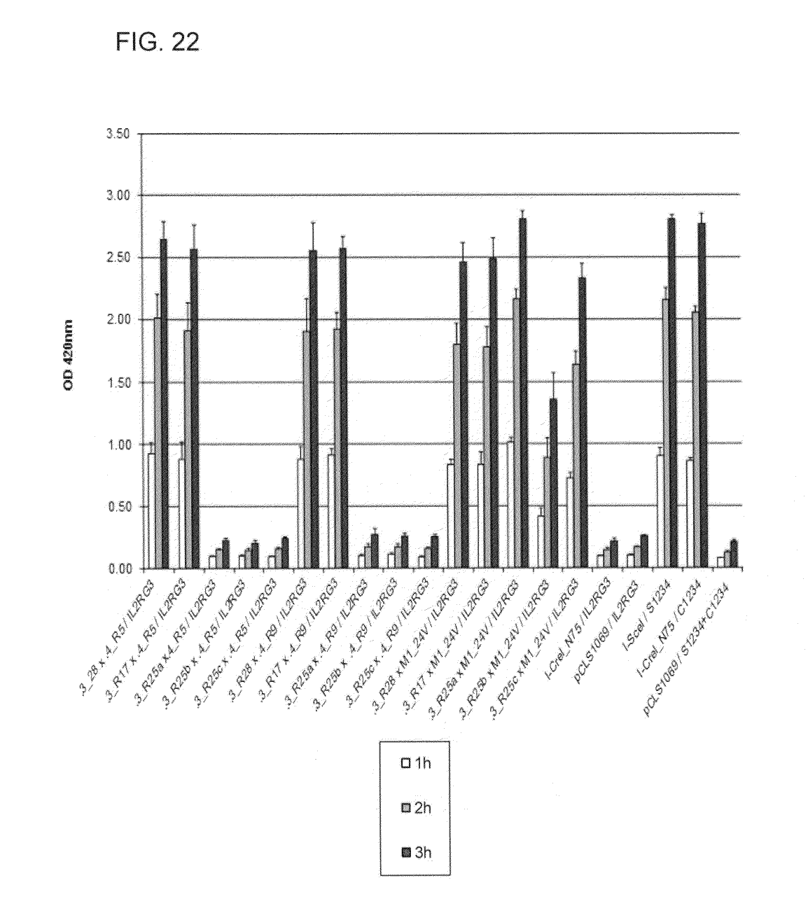

FIG. 22 illustrates cleavage of IL2RG3 target in CHO K1 cells using an extrachromosomal essay. Results of CHO assay for the heterodimers displaying strong cleavage activity against the IL2RG3 target described in example 10. Time course of revelation (OD values are revealed at 3 times: 1 hour (white bars), 2 hours (grey bars) and 3 hours (black bars) after lysis/revelation buffer addition). I-CreI N75, I-SceI and empty vector are used as controls.

FIG. 23 illustrates examples of cleavage of the IL2RG3 target in yeast. Yeast clones expressing M1_{—}24V bearing the amino-acids substitutions described in example 11 are mated with a yeast strain that (i) contains the IL2RG3 target in a reporter plasmid (ii) expresses the .3_R17 or the .3_R28 I-CreI mutant (according to Table IX). In each cluster, the combinations are the following: In lane A: yeast strain containing IL2RG3 target and expressing .3_R28 I-CreI mutant. In lane B: yeast strain containing IL2RG3 target and expressing .3_R17 I-CreI mutant. In column C: yeast clones expressing M1 24V I-CreI mutants with the amino-acids substitutions described in example 11. In column D: yeast clone expressing the M1_{—}24V I-CreI mutant. In column E: yeast clones with positive and negative controls.

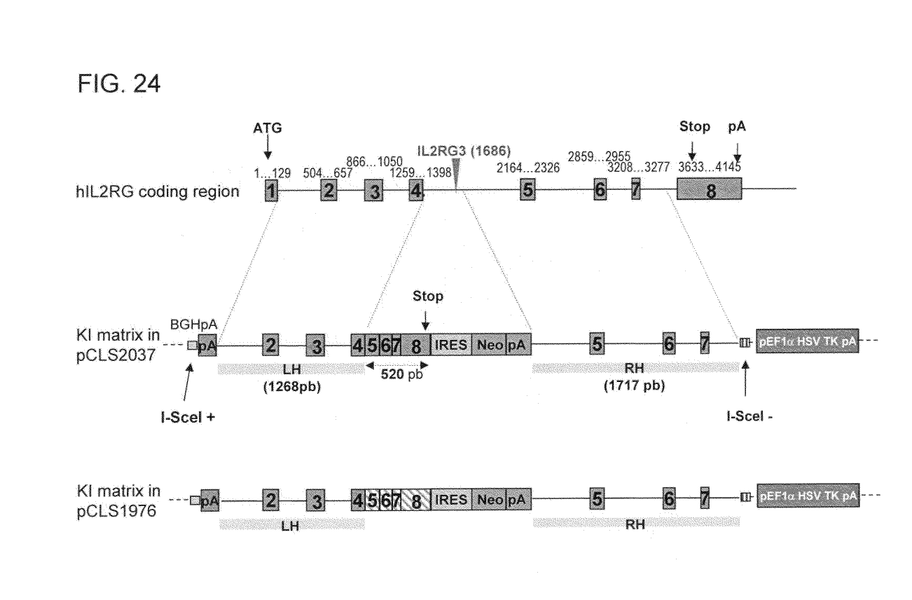

FIG. 24 represents the design of the exons knock-in vectors for targeting of the human IL2RG gene. The structure of the human IL2RG gene is depicted. The gene targeting matrixes are described. LH and RH correspond to the left and right arms of homology. The Neo corresponds to a neomycin CDS. pEF1α HSV TK pA: negative selection cassette. BGHpA: BGH poly adenylation signal. I-SceI +: I-SceI cleavage site in forward orientation, I-SceI −: I-SceI cleavage site in reverse orientation. In pCLS1976, 3% of heterology in nucleotides have been introduced in the cDNA exon 5 to 8.

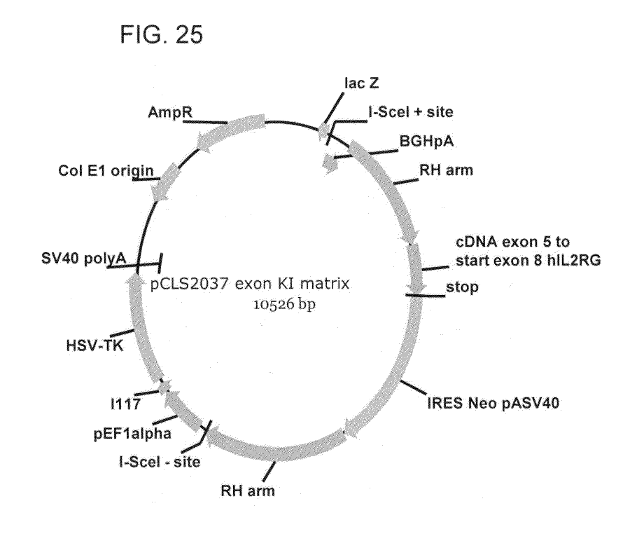

FIG. 25 represents the pCLS2037 plasmid map.

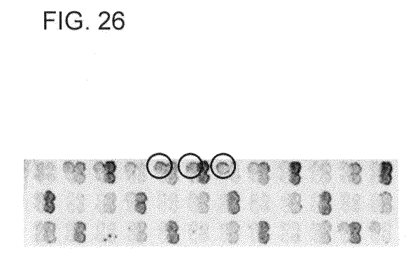

FIG. 26 represents yeast screening of 5AGG_P cutters against the IL2C_P target. Mutants are in the upper left dot of the cluster. The two right dots are experiment internal controls. The three clones that were chosen for further studies are circled.

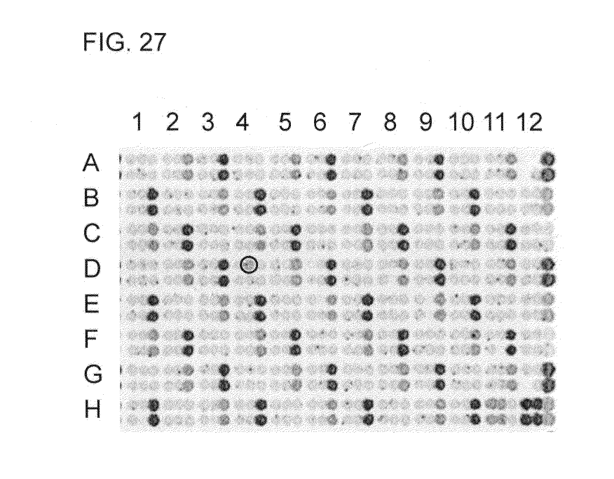

FIG. 27 represents example of primary screening of mutants belonging to the SeqLib1 library against the IL2RG3.4 target. Columns and rows are respectively noted from 1 to 12 and from A to H. In each 6 dots yeast cluster, four SeqLib1 mutants are screened against the IL2RG3.4 target. The two right dots are cluster internal controls. H10, H11 and H12 are also experiment controls. A positive clone is circled.

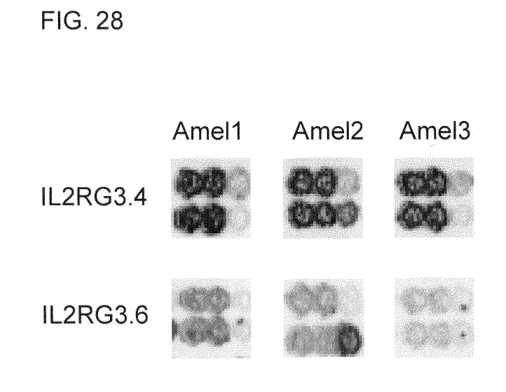

FIG. 28 represents cleavage activity of the three mutants Amel1 to Amel3 toward the IL2RG3.4 and IL2RG3.6 targets. In each 6 dots yeast cluster, the same mutant is screened four times against the same target (four left dots). The upper right dot is the Seq4 mutant and the bottom right dot is an experiment internal control.

### EXAMPLE 1

### Strategy for Engineering Novel Meganucleases Cleaving the Human IL2RG Gene

The combinatorial approach described in Smith et al., Nucleic Acids Res., 2006, 34, e149 and International PCT Applications WO 2007/049095 and WO 2007/057781 and illustrated in FIG. 2D, was used to engineer the DNA binding domain of I-CreI, and cleave a 22 by (non-palindromic) sequence named IL2RG3 and located at position 1686 in intron 4 of the human IL2RG gene (FIGS. 3 and 4). Meganucleases cleaving the IL2RG3 sequence could be used to correct mutations in exon 4 (FIG. 1A). Alternatively, meganucleases cleaving the IL2RG3 sequence could be used to knock-in exonic sequences that would restore a functional IL2RG gene at the IL2RG locus (FIG. 1B). This strategy could be used for any mutation located downstream of the cleavage site.

The IL2RG3 sequence is partly a patchwork of the 10GAC_P, 10GAA_P and 5CTG_P and 5AGG_P targets (FIG. 4), which are cleaved by previously identified meganucleases, obtained as described in International PCT Applications WO 2006/097784, WO 2006/097853, WO 2007/049156 and WO 2007/060495; Arnould et al., J. Mol. Biol., 2006, 355, 443-458 and Smith et al., Nucleic Acids Res., 2006, 34, e149. Thus IL2RG3 could be cleaved by meganucleases combining the mutations found in the I-CreI derivatives cleaving these four targets.

The 10GAC_P, 10GAA_P, 5CTG_P and 5AGG_P sequences are 24 by derivatives of C1221, a palindromic sequence cleaved by I-CreI (International PCT Applications WO 2006/097784, WO 2006/097853, WO 2007/049156 and WO 2007/060495; Arnould et al., J. Mol. Biol., 2006, 355, 443-458 and Smith et al., Nucleic Acids Res., 2006, 34, e149). However, the structure of I-CreI bound to its DNA target suggests that the two external base pairs of these targets (positions −12 and 12) have no impact on binding and cleavage (Chevalier et al., Nat. Struct. Biol., 2001, 8, 312-316; Chevalier B. S. and Stoddard B. L., Nucleic Acids Res., 2001, 29, 3757-3754; Chevalier et al., J. Mol. Biol., 2003, 329, 253-269), and in this study, only positions −11 to 11 were considered. Consequently, the IL2RG3 series of targets were defined as 22 by sequences instead of 24 bp.

IL2RG3 differs from C1221 in 3 out of the 4 by central region. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier B. S. and Stoddard B. L., Nucleic Acids Res., 2001, 29, 3757-3754; Chevalier et al., J. Mol. Biol., 2003, 329, 253-269). Thus, the bases at these positions are not supposed to impact the binding efficiency. However, they could affect cleavage, which results from two nicks at the edge of this region. Thus, the TCTC sequence in −2 to 2 were first substituted with the GTAC sequence from C1221, resulting in target IL2RG3.2 (FIG. 4). Then, two palindromic targets, IL2RG3.3 and IL2RG3.4 were derived from IL2RG3.2. Since IL2RG3.3 and IL2RG3.4 are palindromic, they should be cleaved by homodimeric proteins. Thus, proteins able to cleave the IL2RG3.3 and IL2RG3.4 sequences as homodimers were first designed (examples 2 and 3), and then coexpressed to obtain heterodimers cleaving IL2RG3.2 (example 4). One heterodimer could also cleave IL2RG3 but with a very low cleavage activity. A series of mutants cleaving IL2RG3.3 was chosen and then refined. The chosen mutants were randomly and site-directed mutagenized, and used to form novel heterodimers with a mutant cleaving IL2RG3.4. Heterodimers were screened against the IL2RG3 target (examples 5 and 6) and heterodimers cleaving the IL2RG3 target could be identified, displaying significant cleavage activity. Then, mutant cleaving the IL2RG3.4 target was also refined and used to form novel heterodimers with refined mutants cleaving IL2RG3.3 (examples 7, 8, 10 and 11).

Finally heterodimers were screened against the IL2RG3 target in a single-strand annealing (SSA) based extrachromosomal assay in CHO cells (example 9). Several combinations of I-CreI mutants displayed a very high cleavage activity of the IL2RG3 target, comparable to that of I-SceI against the I-SceI target in the same assay.

### EXAMPLE 2

### Making of Meganucleases Cleaving IL2RG3.3

This example shows that I-CreI mutants can cut the IL2RG3.3 DNA target sequence derived from the left part of the IL2RG3 target in a palindromic form (FIG. 4). Targets sequences described in this example are 22 by palindromic sequences. Therefore, they will be described only by the first 11 nucleotides, followed by the suffix_P. For example, target IL2RG3.3 will be noted also cgacctctggt_P (SEQ ID NO: 13).

IL2RG3.3 is similar to 5CTG_P in positions ±1, ±2, ±3, ±4, ±5, ±9 and ±11 and to 10GAC_P in positions ±1, ±2, ±4, ±8, ±9 ±10 and ±11. It was hypothesized that positions ±6 and ±7 would have little effect on the binding and cleavage activity. Mutants able to cleave 5CTG_P ((t)caaaacctggt_P; SEQ ID NO: 10) were obtained by mutagenesis on I-CreI N75 at positions 24, 42, 44, 68, 70, 75 and 77, as described in Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156. Mutants able to cleave the 10GAC_P target ((t)cgacacgtcgt_P; SEQ ID NO: 15) were obtained by mutagenesis on I-CreI N75 at positions 28, 33, 38, 40 and 70, as described in Smith et al. Nucleic Acids Res., 2006, 34, e149; International PCT Applications WO 2007/060495 and WO 2007/049156.

Both sets of proteins are mutated at position 70. However, the existence of two separable functional subdomains was hypothesized. That implies that this position has little impact on the specificity in base 10 to 8 of the target. Mutations on positions 24 and 42 found in mutants cleaving the 5CTG_P target will be lost during the combinatorial process. But, it was hypothesized that this will have little impact on the capacity of combined mutants to cleave the IL2RG3.3 target.

Therefore, to check whether combined mutants could cleave the IL2RG3.3 target, mutations at positions 44, 68, 70, 75 and 77 from proteins cleaving 5CTG_P were combined with the 28, 33, 38 and 40 mutations from proteins cleaving 10GAC_P.

### 1) Material and Methods

The method for producing meganuclease variants and the assays based on cleavage-induced recombination in mammal or yeast cells, which are used for screening variants with altered specificity are described in the International PCT Application WO 2004/067736; Epinat et al., Nucleic Acids Res., 2003, 31, 2952-2962; Chames et al., Nucleic Acids Res., 2005, 33, e178, and Arnould et al., J. Mol. Biol., 2006, 355, 443-458. These assays result in a functional LacZ reporter gene which can be monitored by standard methods.

### a) Construction of Target Vector

The target was cloned as follow: oligonucleotide corresponding to the target sequence flanked by gateway cloning sequence was ordered from PROLIGO: 5′tggcatacaagtttcgacctctggtaccagaggtcgacaatcgtctgtca3′ (SEQ ID NO: 16). Double-stranded target DNA, generated by PCR amplification of the single stranded oligonucleotide, was cloned using the Gateway protocol (INVITROGEN) into yeast reporter vector (pCLS1055, FIG. 5). Yeast reporter vector was transformed into Saccharomyces cerevisiae strain FYBL2-7B (MAT a, ura3Δ851, trp1Δ63, leu2Δ1, lys2Δ202).

### b) Construction of Combinatorial Mutants

I-CreI mutants cleaving 10GAC_P or 5CTG_P were identified as described in Smith et al. Nucleic Acids Res., 2006, 34, e149; International PCT Applications WO 2007/060495 and WO 2007/049156, and Arnould et al., J. Mol. Biol., 2006, 355, 443-458; International PCT Applications WO 2006/097784 and WO 2006/097853, respectively for the 10GAC_P and 5CTG_P targets. In order to generate I-CreI derived coding sequence containing mutations from both series, separate overlapping PCR reactions were carried out that amplify the 5′ end (aa positions 1-43) or the 3′ end (positions 39-167) of the I-CreI coding sequence. For both the 5′ and 3′ end, PCR amplification is carried out using primers Gal10F 5′-gcaactttagtgctgacacatacagg-3′ (SEQ ID NO: 17) or Gal10R 5′-acaaccttgattggagacttgacc-3′ (SEQ ID NO: 18) specific to the vector (pCLS0542, FIG. 6) and primers assF 5′-ctannnttgaccttt-3′ (SEQ ID NO: 19) or assR 5′-aaaggtcaannntag-3′(SEQ ID NO: 20) where nnn code for residue 40, specific to the I-CreI coding sequence for amino acids 39-43. The PCR fragments resulting from the amplification reaction realized with the same primers and with the same coding sequence for residue 40 were pooled. Then, each pool of PCR fragments resulting from the reaction with primers Gal10F and assR or assF and Gal10R was mixed in an equimolar ratio. Finally, approximately 25 ng of each final pool of the two overlapping PCR fragments and 75 ng of vector DNA (pCLS0542) linearized by digestion with NcoI and EagI were used to transform the yeast Saccharomyces cerevisiae strain FYC2-6A (MATα, trp1Δ63, leuΔ1, his3Δ200) using a high efficiency LiAc transformation protocol (Gietz and Woods, Methods Enzymol., 2002, 350, 87-96). An intact coding sequence containing both groups of mutations is generated by in vivo homologous recombination in yeast.

### c) Mating of Meganuclease Expressing Clones and Screening in Yeast

Screening was performed as described previously (Arnould et al., J. Mol. Biol., 2006, 355, 443-458). Mating was performed using a colony gridder (QpixII, Genetix). Mutants were gridded on nylon filters covering YPD plates, using a low gridding density (about 4 spots/cm²). A second gridding process was performed on the same filters to spot a second layer consisting of different reporter-harboring yeast strains for each target. Membranes were placed on solid agar YPD rich medium, and incubated at 30° C. for one night, to allow mating. Next, filters were transferred to synthetic medium, lacking leucine and tryptophan, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### d) Sequencing of Mutants

To recover the mutant expressing plasmids, yeast DNA was extracted using standard protocols and used to transform E. coli. Sequence of mutant ORF were then performed on the plasmids by MILLEGEN SA. Alternatively, ORFs were amplified from yeast DNA by PCR (Akada et al., Biotechniques, 2000, 28, 668-670), and sequence was performed directly on PCR product by MILLEGEN SA.

### 2) Results

I-CreI combinatorial mutants were constructed by associating mutations at positions 44, 68, 70, 75 and 77 from proteins cleaving 5CTG_P with the 28, 33, 38 and 40 mutations from proteins cleaving 10GAC_P on the I-CreI scaffold, resulting in a library of complexity 264. Combinations are displayed on Table II. This library was transformed into yeast and 864 clones (3.3 times the diversity) were screened for cleavage against the IL2RG3.3 DNA target (cgacctctggt_P; SEQ ID NO: 13). A total of 14 positive clones were found and examples of positives are shown in FIG. 7.

Each positive yeast strain may express several I-CreI combinatorial mutants. Mutant expressing plasmids were recovered from positive clones and used to transform E. coli. Three clones for each were sequenced and retransformed in yeast to validate the cleavage of the target by each monoclonal mutant expressing yeast strain. After validation by screening and sequencing of the mutant meganucleases ORF, the 14 positive clones turned out to correspond to 20 different novel endonucleases cleaving the IL2RG3.3 target (named ml to m20; SEQ ID NO: 48, 115, 49 to 65, respectively). Five correspond to expected combination of mutations (Table II). The fifteen others are I-CreI combined mutants in which additional mutations were also identified. Such mutants likely result from PCR artefacts during the combinatorial process (see materials and methods). Alternatively, the mutants having additional mutations may be I-CreI combined mutants resulting of micro recombination between two original mutants during the in vivo homologous recombination in yeast (Table III).

TABLE II
Cleavage of the IL2RG3.3 target by the panel of variants
theoretically present in the combinatorial library

| Amino | | | | | | | | | | | | |
|---|---|---|---|---|---|---|---|---|---|---|---|---|
| acids at | | | | | | | | | | | | |
| positions | | | | | | | | | | | | |
| 44, 68, 70, | | | | | | | | | | | | |
| 75 and 77 | | | | | | | | | | | | |
| (ex: RYSEK | | | | | | | | | | | | |
| stands for | | | | | | | | | | | | |
| R44, Y68, | | | | | | | | | | | | |
| S70, | Amino acids at positions 28, 33, 38 and 40 | | | | | | | | | | | |
| E75 and | (ex: KHQS stands for K28, H33, Q38 and S40) | | | | | | | | | | | |
| K77) | KHQS | TRQR | ARQR | KRQY | KRQS | KRQQ | RRYQ | KRQA | ARYR | RYHH | SRQR | KRQE |
| RYSEK | | | | | | | | | | | | |
| QRSNQ | | | | | | | | | | | | |
| RYSEQ | | | | | | | | | | | | |
| RYSEV | | | | +m5 | | | | | | | | |
| RYSDT | | | | +m19 | | | | +m12 | | | | |
| RESER | | | | | | | | | | | | |
| RISER | | | | | | | | | | | | |
| RQSER | | | | | | | | | | | | |
| KTSDV | | | | | | | | | | | | |
| RASNN | | | | | | | | | | | | |
| RRSDY | | | | | | | | | | | | |
| RYSER | | | | | | | | | | | | |
| RYSNI | | | | | | | | | | | | |
| RNSER | | | | | | | | | | | | |
| RRSEY | | | | | | | | | | | | |
| RYSET | | | | | | | | | | | | |
| RYSQY | | | | | | | | | | | | |
| RYSEI | | | | | | | | | | | | |
| RYSDQ | | | | | +m2 | | | +m11 | | | | |
| KYSQT | | | | | | | | | | | | |
| QRSNN | | | | | | | | | | | | |
| RRSNY | | | | | | | | | | | | |

+indicates that a functional combinatorial mutant cleaving the IL2RG3.3 target was found among the identified positives.

TABLE III
I-CreI combined mutants with additional
mutations cleaving the IL2RG3.3 target

| | Amino acids at positions |
|---|---|
| Mutant | 28, 33, 38, 40/44, 68, 70, 75, 77 |
| m1 | KHQS/KYSEQ |
| m3 | KHQS/RYSDQ + 143I 163L |
| m4 | TRQR/KYSEV |
| m6 | KRQQ/KYSQY |
| m7 | KHQS/KYSEV |
| m8 | KRQR/RYSDT |
| m9 | KRQR/RYSDQ + 132V |
| m10 | KRQY/RYSDT + 132V |
| m13 | KRQA/RYSEV + 132T |
| m14 | KRQS/RYSDH |
| m15 | TPQR/KYSEV |
| m16 | KRQY/RYSDV |
| m17 | KHQS/KYSEV + 31R |
| m18 | KHQS/KYSET |
| m20 | KRQA/RYSDV |

### EXAMPLE 3

### Making of Meganucleases Cleaving IL2RG3.4

This example shows that I-CreI variant can cleave the IL2RG3.4 DNA target sequence derived from the right part of the IL2RG3 target in a palindromic form (FIG. 4). All targets sequences described in this example are 22 by palindromic sequences. Therefore, they will be described only by the first 11 nucleotides, followed by the suffix P. For example, IL2RG3.4 will be called tgaaccagggt_P (SEQ ID NO: 14).

IL2RG3.4 is similar to 5AGG_P in positions ±1, ±2, ±3, ±4, ±5, ±6, ±8 and ±9 and to 10GAA_P in positions ±1, ±2, ±6, ±8, ±9 and ±10. It was hypothesized that positions ±7 and ±11 would have little effect on the binding and cleavage activity. Mutants able to cleave 5AGG_P were obtained by mutagenesis on I-CreI N75 at positions 24, 44, 68, 70, 75 and 77, as described in Arnould et al., J. Mol. Biol., 2006, 355, 443-458; Smith et al. Nucleic Acids Res., 2006, 34, e149; International PCT Applications WO 2006/097784, WO 2006/097853, WO 2007/060495 and WO 2007/049156. Mutants able to cleave the 10GAA P target were obtained by mutagenesis on I-CreI N75 and D75 at positions 30, 32, 33, 38 and 40, as described in Smith et al. Nucleic Acids Res., 2006, 34, e149; International PCT Applications WO 2007/060495 and WO 2007/049156. Mutations at positions 24 found in mutants cleaving the 5AGG_P target will be lost during the combinatorial process. But, it was hypothesized that this will have little impact on the capacity of combined mutants to cleave the IL2RG3.4 target.

To check whether combined mutants could cleave the IL2RG3.4 target, mutations at positions 44, 68, 70, 75 and 77 from proteins cleaving 5AGG_P ((t)caaaacagggt_P (SEQ ID NO: 11)) were combined with the 30, 32, 33, 38 and 40 mutations from proteins cleaving 10GAA_P ((t)cgaaacgtcgt_P (SEQ ID NO: 114))

### 1) Material and Methods

The experimental procedures are described in example 2.

### 2) Results

I-CreI combinatorial mutants were constructed by associating mutations at positions 44, 68, 70, 75 and 77 from proteins cleaving 5AGG_P with the 30, 32, 33, 38 and 40 mutations from proteins cleaving 10GAA_P on the I-CreI scaffold, resulting in a library of complexity 4160. Examples of combinatorial mutants are displayed on Table IV. This library was transformed into yeast and 8064 clones (1.9 times the diversity) were screened for cleavage against the IL2RG3.4 DNA target (tgaaccagggt_P (SEQ ID NO: 14)). Three positives clones were found (two strong cutters and one weak cutter), which after sequencing and validation by secondary screening (as in example 2) turned out to correspond to two different novel endonucleases: M1 (SEQ ID NO: 45) and M2 (SEQ ID NO: 66), (Table V). M1 cleavage of IL2RG3.4 target is shown in FIG. 8. The two novel endonucleases are I-CreI combined mutants resulting from micro recombination between two original mutants during the in vivo homologous recombination in yeast. And M2 has an additional mutation (54L) probably due to PCR artefacts during the combinatorial process.

TABLE IV
Panel of mutants* theoretically presents
in thecombinatorial library

| Amino acids at | | | | | | | | |
|---|---|---|---|---|---|---|---|---|
| positions | | | | | | | | |
| 44, 68, 70, 75 and 77 | Amino acids at positions 30, 32, 33, | | | | | | | |
| (ex: ARSER stands | 38 and 40 (ex: NSHQS stands for N30, | | | | | | | |
| for A44, R68, S70, | S32, H33, Q38 and S40) | | | | | | | |
| E75 and R77) | NSHQS | RDYQS | RTYQS | NEYQS | NSHSS | KSAQS | KSSQS | RSCTS |
| ARSER | | | | | | | | |
| TRSER | | | | | | | | |
| TYSER | | | | | | | | |
| RYSEV | | | | | | | | |
| RYSET | | | | | | | | |
| TRSYI | | | | | | | | |
| YRSQV | | | | | | | | |
| YRSQI | | | | | | | | |
| ARSYV | | | | | | | | |
| ARSYY | | | | | | | | |
| HRSDI | | | | | | | | |
| NRSYI | | | | | | | | |
| SRSYN | | | | | | | | |
| YSSQV | | | | | | | | |

*Only 112 out of the 4160 combinations are displayed. None of them were identified in the positive clones

TABLE V
Sequence of mutants cleaving the
IL2RG3.4 target.

| | Amino acids at positions |
|---|---|
| Mutant | 30, 32, 33, 38, 40/44, 68, 70, 75, 77 |
| M1 | RTYQS/AYSER |
| M2 | KSCQS/TRSER + 54L |

### EXAMPLE 4

### Making of Meganucleases Cleaving IL2RG3.2

I-CreI mutants able to cleave each of the palindromic IL2RG3 derived targets (IL2RG3.3 and IL2RG3.4) were identified in examples 2 and 3. Pairs of such mutants (one cutting IL2RG3.3 and one cutting IL2RG3.4) were co-expressed in yeast. Upon co-expression, there should be three active molecular species, two homodimers, and one heterodimer. It was assayed whether the heterodimers that should be formed cut the non palindromic IL2RG3 and IL2RG3.2 DNA targets.

### 1) Material and Methods

### a) Cloning of Mutants in Kanamycin Resistant Vector

To coexpress two I-CreI mutants in yeast, mutants cutting the IL2RG3.3 sequence were subcloned in a yeast expression vector marked with a kanamycin resistance gene (pCLS1107, FIG. 9). Mutants were amplified by PCR reaction using primers common for vectors pCLS0542 and pCLS1107 (Gal10F 5′-gcaactttagtgctgacacatacagg-3′ (SEQ ID NO: 17) and Gal10R 5′-acaaccttgattggagacttgacc-3 ‘(SEQ ID NO: 18). Approximately 25 ng of PCR fragment and 25 ng of DNA vector (pCLS1107) linearized by digestion with DraIII and NgoMIV are used to transform the yeast Saccharomyces cerevisiae strain FYC2-6A (MATα, trp1Δ63, leu2Δ1, his3Δ200) using a high efficiency LiAc transformation protocol. An intact coding sequence for the I-CreI mutant is generated by in vivo homologous recombination in yeast.

Each yeast strain containing a mutant cutting the IL2RG3.3 target subcloned in vector pCLS 1107 was then mated with yeast expressing the IL2RG3.3 target to validate it. To recover the mutant expressing plasmids, yeast DNA was extracted using standard protocols. Then, E. coli was transformed by yeast DNA to prepare bacterial DNA.

### b) Mutants Coexpression

Yeast strain expressing a mutant cutting the IL2RG3.4 target in pCLS0542 expression vector was transformed with DNA coding for a mutant cutting the IL2RG3.3 target in pCLS 1107 expression vector. Transformants were selected on −L Glu medium containing G418.

### c) Mating of Meganucleases Coexpressing Clones and Screening in Yeast

Mating was performed using a colony gridder (QpixII, Genetix). Mutants were gridded on nylon filters covering YPD plates, using a low gridding density (about 4 spots/cm²). A second gridding process was performed on the same filters to spot a second layer consisting of different reporter-harbouring yeast strains for each target. Membranes were placed on solid agar YPD rich medium, and incubated at 30° C. for one night, to allow mating. Next, filters were transferred to synthetic medium, lacking leucine and tryptophan, adding G418, with galactose (2%) as a carbon source, and incubated for five days at 37° C., to select for diploids carrying the expression and target vectors. After 5 days, filters were placed on solid agarose medium with 0.02% X-Gal in 0.5 M sodium phosphate buffer, pH 7.0, 0.1% SDS, 6% dimethyl formamide (DMF), 7 mM β-mercaptoethanol, 1% agarose, and incubated at 37° C., to monitor β-galactosidase activity. Results were analyzed by scanning and quantification was performed using appropriate software.

### 2) Results

Co-expression of mutants cleaving the IL2RG3.3 target (17 chosen mutants described in Tables II and III) and the two mutants cleaving the IL2RG3.4 target (described in Table V) resulted in efficient cleavage of the IL2RG3.2 target in all the cases (screen examples are shown in FIG. 10A). All combinations tested are summarized in Table VI. However, only one out of these combinations is able to cut very weakly the IL2RG3 natural target (FIG. 10B and Table VI). IL2RG3 differs from the IL2RG3.2 sequence just by 3 by in positions −2, −1 and 1 (FIG. 4).

TABLE VI
Combinations that resulted in cleavage of the IL2RG3.2 target

| | | Mutants cutting IL2RG3.4 | |
|---|---|---|---|
| | | amino acids at positions 30, 32, | |
| Mutants cutting IL2RG3.3 | | 33, 38, 40/44, 68, 70, 75 and 77 | |
| amino acids at positions 28, 33, | | (ex: RTYQS/AYSER stands for R30, T32, | |
| 38, 40/44, 68, 70, 75, 77 | | Y33, Q38, S40/A44,Y68, S70, E75 and R77) | |
| (ex: KHQS/KYSEQ stands for K28, H33, | | M1 | M2 |
| Q38, S40/K44, Y68, S70, E75 and Q77) | | RTYQS/AYSER | KSCQS/TRSER + 54L |
| m1 | KHQS/KYSEQ | + | + |
| m2 | KRQS/RYSDQ | + | + |
| m3 | KHQS/RYSDQ + 143I 163L | + | + |
| m4 | TRQR/KYSEV | + | + |
| m5 | KRQY/RYSEV | + | + |
| m6 | KRQQ/KYSQY | + | + |
| m7 | KHQS/KYSEV | + | + |
| m8 | KRQR/RYSDT | + | + |
| m9 | KRQR/RYSDQ + 132V | + | + |
| m10 | KRQY/RYSDT + 132V | +* | + |
| m11 | KRQA/RYSDQ | + | + |
| m12 | KRQA/RYSDT | + | + |
| m13 | KRQA/RYSEV + 132T | + | + |
| m14 | KRQS/RYSDH | + | + |
| m17 | KHQS/KYSEV + 31R | + | + |
| m18 | KHQS/KYSET | + | + |
| m19 | KRQY/RYSDT | + | + |

+ indicates that the heterodimeric mutant cleaved the IL2RG3.2 target.
*indicates that the combination weakly cuts the IL2RG3 target.

### EXAMPLE 5

### Making of Meganucleases Cleaving IL2RG3 by Random Mutagenesis of Proteins Cleaving IL2RG3.3 and Assembly with Protein Cleaving IL2RG3.4

I-CreI mutants able to cleave the non palindromic IL2RG3.2 target were previously identified by assembly of mutants cleaving the palindromic IL2RG3.3 and IL2RG3.4 targets. However, none of these combinations was able to cleave efficiency IL2RG3, which differs from IL2RG3.2 only by 3 by in positions −2, −1 and 1. The weak signal observed for one of the combinations of mutants is not sufficient.

Therefore, the protein combinations cleaving IL2RG3.2 were mutagenized, and variants cleaving IL2RG3 efficiently were screened. According to the structure of the I-CreI protein bound to its target, there is no contact between the 4 central base pairs (positions −2 to 2) and the I-CreI protein (Chevalier et al., Nat. Struct. Biol., 2001, 8, 312-316; Chevalier B. S. and Stoddard B. L., Nucleic Acids Res., 2001, 29, 3757-3754; Chevalier et al., J. Mol. Biol., 2003, 329, 253-269). Thus, it is difficult to rationally choose a set of positions to mutagenize, and mutagenesis was done on the C-terminal part of the protein (83 last amino acids) or on the whole protein. Random mutagenesis results in high complexity libraries, and the complexity of the variants libraries to be tested was limited by mutagenizing only on one of the two components of the heterodimers cleaving IL2RG3.2.

Thus, proteins cleaving IL2RG3.3 were mutagenized, and it was tested whether they could cleave IL2RG3 efficiently when coexpressed with a protein cleaving IL2RG3.4.

### 1) Material and Methods

### a) Random Mutagenesis

Random mutagenesis were created on a pool of chosen mutants by PCR using Mn² or derivatives of dNTPs as 8-oxo-dGTP and dPTP, in two-step PCR process as described in the protocol from JENA BIOSCIENCE GmbH in JBS dNTP-Mutagenesis kit.

For random mutagenesis on the whole protein primers used are preATGCreFor (5′-gcataaattactatacttctatagacacgcaaacacaaatacacagcggccttgccacc-3′; SEQ ID NO: 21) and ICreIpostRev (5′-ggctcgaggagctcgtctagaggatcgctcgagttatcagtcggccgc-3′; SEQ ID NO: 22). For random mutagenesis on the C-terminal part of the protein primer used are AA78a83For (5′-ttaagcgaaatcaagccg-3′; SEQ ID NO: 23) and ICreIpostRev with dNTPs derivatives; the rest of the protein is amplified with a high fidelity taq polymerase and without dNTPs derivatives using primers preATGCreFor and AA78a83Rev (5′-cggcttgatttcgcttaa-3′; SEQ ID NO: 24).

Pools of mutants were amplified by PCR reaction using these primers common for the pCLS0542 (FIG. 6) and pCLS1107 (FIG. 9) vectors. Approximately 75 ng of PCR fragment and 75 ng of vector DNA (pCLS1107) linearized by digestion with DraIII and NgoMIV are used to transform the yeast Saccharomyces cerevisiae strain FYC2-6A (MATα, trp1Δ63, leu2Δ1, his3Δ200) using a high efficiency LiAc transformation protocol. A library of intact coding sequence for the I-CreI mutant is generated by in vivo homologous recombination in yeast. Positives resulting clones were verified by sequencing (MILLEGEN).

b) Cloning of Mutants in Vector pCLS0542 in the Yeast Strain Containing the IL2RG3 Target

The yeast strain FYBL2-7B (MAT a, ura3Δ851, trp1Δ63, leu2Δ1, lys2Δ202) containing the IL2RG3 target into yeast reporter vector (pCLS1055, FIG. 5) is transformed with a mutant cutting IL2RG3.4 target in pCLS0542 vector, using a high efficiency LiAc transformation protocol. Mutant-target yeasts are used as targets for mating assays as described in example 4.

### 2) Results

New I-CreI mutants able to efficiently cleave IL2RG3 target when forming heterodimers with a mutant cleaving the IL2RG3.4 target, were identified.

Eight mutants cleaving IL2RG3.3 (m1/m3/m7/m10/m14/m17/m18/m19 according to Tables II and III) were pooled, randomly mutagenized on all proteins or on the C-terminal part of proteins and transformed into yeast. 8928 transformed clones were then mated with a yeast strain that (i) contains the IL2RG3 target in a reporter plasmid (ii) expresses the M1 mutant (RTYQS/AYSER according to Table V), a variant cleaving the IL2RG3.4 target described in example 3. 6 clones (SEQ ID NO: 67 to 72) described in Table VII, were found to trigger cleavage of the IL2RG3 target when mated with such yeast strain.

TABLE VII
Functional mutant combinations displaying
cleavage activity for IL2RG3 DNA target

| Mutant | |
|---|---|
| cleaving | Optimized* I-CreI mutants derived from |
| IL2RG3.4 | mutants cleaving IL2RG3.3 round 1 |
| RTYQS/ | 31R 33H 44K 68Y 70S 75E 77V 80G 154G |
| AYSER | 157G 31R 33H 44K 68Y 70S 71R 75E 77V |
| M1 | 4E 33R 39I 40Y 44R 68Y 70S 75D 77T 87L |
| | 132V 162P 31R 33H 44K 68Y 70S 75E 77V |
| | 139R 19S 33H 40Y 44R 68Y 70S 75D 77T |
| | 26R 31R 33H 44K 68Y 70S 75E 77V |

*Mutations resulting from random mutagenesis are in bold.

Those 6 optimized clones were subjected to a second round of optimization. They were pooled, randomly mutagenized on all proteins or on the C-terminal part of proteins and transformed into yeast. 4464 transformed clones were then mated with a yeast strain that (i) contains the IL2RG3 target in a reporter plasmid (ii) expresses the M1 mutant (RTYQS/AYSER according to Table V), a variant cleaving the IL2RG3.4 target described in example 3. 102 clones were found to trigger an efficient cleavage of the IL2RG3 target when mated with such yeast strain. Examples of positives are shown on FIG. 11.

The sequence of the 11 best I-CreI mutants (SEQ ID NO: 73 to 83) cleaving the IL2RG3 target when forming heterodimer with the M1 mutant (RTYQS/AYSER according to Table V) are listed in Table VIII.

TABLE VIII
Functional I-CreI mutant combinations displaying strong cleavage
activity for IL2RG3 DNA target

| Mutant | Optimized I-Crel mutants derived from mutants | |
|---|---|---|
| cleaving | cleaving IL2RG3.3 round 2 | |
| IL2RG3.4 | name | sequence |
| M1 | .3_R1 | 26R 31R 33H 44K 68Y 70S 75E 77V 89A 117G 139R |
| RTYQS/ | .3_R2 | 26R 31R 33H 44K 68Y 70S 75E 77V 139R |
| AYSER | .3_R3 | 26R 31R 33H 39I 44K 68Y 70S 75E 77V 82R 139R |
| | .3_R4 | 26R 31R 33H 39I 44K 46A 68Y 70S 71R 75E 77V |
| | .3_R5 | 26R 31R 33H 39I 40Y 44R 68Y 70S 75D 77T 87L 132V 162P |
| | .3_R6 | 7E 26R 31R 33H 44K 68Y 70S 75E 77V 139R |
| | .3_R7 | 26R 31R 33H 44K 68Y 70S 75E 77V 111R 139R |
| | .3_R8 | 2D 26R 31R 33H 44K 68Y 70S 75E 77V 80G 121R 139R |
| | .3_R9 | 26R 31R 33H 44K 68Y 70S 75E 77V 139R 159R |
| | .3_R10 | 19S 33H 40Y 43L 44R 68Y 70S 75D 77T 132V 159E 160G 162F |
| | .3_R11 | 19S 33H 40Y 44K 68Y 70S 71R 75E 77V |

### EXAMPLE 6

### Making of Meganucleases Cleaving IL2RG3 by Site-Directed Mutagenesis of Protein Cleaving IL2RG3.3 and Assembly with Proteins Cleaving IL2RG3.4

The initial and optimized I-CreI mutants (round 1) cleaving IL2RG3.3 described in Tables II, III and VII was mutagenized by introducing selected amino-acids substitutions in the proteins and screening for more efficient variants cleaving IL2RG3 in combination with the M1 mutant cleaving IL2RG3.4 identified in example 3.

Five amino-acid substitutions have been found in previous studies to enhance the activity of I-CreI derivatives: these mutations correspond to the replacement of Glycine 19 with Serine (G195), Phenylalanine 54 with Leucine (F54L), Phenylalanine 87 with Leucine (F87L), Valine 105 with Alanine (V105A) and Isoleucine 132 with Valine (I132V). These mutations were individually introduced into the coding sequence of proteins cleaving IL2RG3.3, and the resulting proteins were tested for their ability to induce cleavage of the IL2RG3 target, upon co-expression with mutant cleaving IL2RG3.4.

### 1) Material and Methods

### Site-Directed Mutagenesis

Site-directed mutagenesis libraries were created by PCR on a pool of the twenty initial mutants m1 to m20 cleaving IL2RG3.3 (example 2; Tables II and III) and the six optimized mutants cleaving IL2RG3.3 described in Table VII (example 5). For example, to introduce the G19S substitution into the coding sequences of the mutants, two separate overlapping PCR reactions were carried out that amplify the 5′ end (residues 1-24) or the 3′ end (residues 14-167) of the I-CreI N75 coding sequence. For both the 5′ and 3′ end, PCR amplification is carried out using a primer with homology to the vector [Gal10F 5′-gcaactttagtgctgacacatacagg-3′ (SEQ ID NO: 17) or Gal10R 5′-acaaccttgattggagacttgacc-3′ (SEQ ID NO: 18)] and a primer specific to the I-CreI coding sequence for amino acids 14-24 that contains the substitution mutation G19S [G19SF 5′-gccggctttgtggactctgacggtagcatcatc-3′ (SEQ ID NO: 25) or G19SR 5′-gatgatgctaccgtcagagtccacaaagccggc-3′(SEQ ID NO: 26)]. The resulting PCR products contain 33 by of homology with each other. The PCR fragments were purified. Finally, approximately 25 ng of each of the two overlapping PCR fragments and 75 ng of vector DNA (pCLS 1107) linearized by digestion with DraIII and NgoMIV were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). Intact coding sequences containing the G19S substitution are generated by in vivo homologous recombination in yeast.

The same strategy is used with the following pair of oligonucleotides to create the other libraries containing the F54L, F87L, V105A and I132V substitutions, respectively:

* F54LF:
(SEQ ID NO: 27)
5′-acccagcgccgttggctgctggacaaactagtg-3′
and
F54LR:
(SEQ ID NO: 28)
5′-cactagtttgtccagcagccaacggcgctgggt-3′;
* F87LF:
(SEQ ID NO: 29)
5′-aagccgctgcacaacctgctgactcaactgcag-3′
and
F87LR:
(SEQ ID NO: 30)
5′-ctgcagttgagtcagcaggttgtgcagcggctt-3′;
* V105AF:
(SEQ ID NO: 31)
5′-aaacaggcaaacctggctctgaaaattatcgaa-3′
and
V105AR:
(SEQ ID NO: 32)
5′-ttcgataattttcagagccaggtttgcctgttt-3′;
* I132VF:
(SEQ ID NO: 33)
5′-acctgggtggatcaggttgcagctctgaacgat-3′
and
I132VR:
(SEQ ID NO: 34)
5′-atcgttcagagctgcaacctgatccacccaggt-3′.

### 2) Results

Libraries containing the five amino-acids substitutions (Glycine 19 with Serine, Phenylalanine 54 with Leucine, Phenylalanine 87 with Leucine, Valine 105 with Alanine and Isoleucine 132 with Valine) were constructed on a pool of 26 I-CreI mutants (described in Tables II, III and VII). 372 transformed clones for each library were then mated with a yeast strain that (i) contains the IL2RG3 target in a reporter plasmid (ii) expresses the M1 mutant (RTYQS/AYSER according to Table V), a variant cleaving the IL2RG3.4 target described in example 3.

New I-CreI mutants able to efficiently cleave IL2RG3 target when forming heterodimers with a mutant cleaving the IL2RG3.4 target were identified.

A total of 123 clones were found to trigger cleavage of the IL2RG3 target when mated with such yeast strain. Examples of positives are shown on FIG. 12.

The sequence of the 17 best I-CreI mutants (SEQ ID NO: 84 to 100) cleaving the IL2RG3 target when forming heterodimer with the M1 mutant (RTYQS/AYSER according to Table V) are listed in Table IX. Those I-CreI mutants are expected mutants due to the site-directed mutagenesis, but also contain unexpected mutations probably due to the PCR reaction and micro-recombination between two mutants of the pool used for the libraries construction.

TABLE IX
Functional mutant combinations displaying strong
cleavage activity for IL2RG3 DNA target

| Mutant | Optimized mutants derived from mutants | |
|---|---|---|
| cleaving | cleaving IL2RG3.3 | |
| IL2RG3.4 | Name | Sequence |
| M1 | .3_R12 | 19S 26R 31R 33H 44K 68Y 70S 75E 77V |
| RTYQS/ | .3_R13 | 19S 31R 33H 44K 68Y 70S 75E 77V 139R |
| AYSER | .3_R14 | 19S 33H 40Y 44K 68Y 70S 75E 77V 139R |
| | .3_R15 | 8G 19S 26R 31R 33H 44K 68Y 70S 75E 77V 139R |
| | .3_R16 | 19S 33R 40Y 44R 68Y 70S 75E 77V |
| | .3_R17 | 26R 31R 33H 44K 54L 68Y 70S 75E 77V 139R |
| | .3_R18 | 31R 33H 44K 68Y 70S 71R 75E 77V 87L 132V 139R 147A |
| | .3_R19 | 19S 33H 40Y 44R 68Y 70S 75D 77T 87L 139R |
| | .3_R20 | 33R 40Y 44R 68Y 70S 75D 77T 87L |
| | .3_R21 | 19S 33H 40Y 44R 68Y 70S 75D 77T 87L 154G 157G |
| | .3_R22 | 31R 33H 44K 68Y 70S 75E 77V 80G 105A 139R |
| | .3_R23 | 19S 33H 40Y 44R 68Y 70S 75D 77T 132V |
| | .3_R24 | 19S 33H 40Y 44R 68Y 70S 75D 77T 132V 154G |
| | .3_R25 | 19S 33H 40Y 44R 68Y 70S 75E 77V 132V |
| | .3_R26 | 31R 33H 44K 68Y 70S 71R 75E 77V 132V |
| | .3_R27 | 31R 33H 44K 68Y 70S 75E 77V 80G 132V 139R |
| | .3_R28 | 31R 33H 44K 68Y 70S 75E 77V 132V 139R |

### EXAMPLE 7

### Refinement of Meganucleases Cleaving the IL2RG3 Target Site by Site-Directed Mutagenesis of the Mutant Cleaving IL2RG3.4

I-CreI mutants able to cleave the IL2RG3 target were previously identified by assembly of a mutant cleaving IL2RG3.4 and refined mutants cleaving IL2RG3.3. To increase the activity of the meganucleases, the second component of the heterodimers cleaving IL2RG3 was mutagenized. Therefore, the mutant cleaving IL2RG3.4 was mutagenized and variants cleaving IL2RG3 more efficiently in combination with the refined mutants cleaving IL2RG3.3 identified in examples 5 and 6, were screened.

Two single amino acid substitutions (Glycine-19 with Serine and Isoleucine-132 with Valine) were introduced. Those amino-acids substitutions, were previously found to increase the cleavage activity of I-CreI derived meganucleases (see example 6). The mutations were incorporated into the M1 mutant (RTYQS/AYSER according to Table V) cleaving the IL2RG3.4 target.

### 1) Material and Methods

### a) Site-Directed Mutagenesis

To introduce the G19S substitution into the M1 mutant coding sequence (RTYQS/AYSER according to Table V), two separate overlapping PCR reactions were carried out that amplify the 5′ end (residues 1-24) or the 3′ end (residues 14-167) of the I-CreI coding sequence. For both the 5′ and 3′ end, PCR amplification is carried out using a primer with homology to the vector [Gal10F 5′-gcaactttagtgctgacacatacagg-3′ (SEQ ID NO: 17) or Gal10R 5′-acaaccttgattggagacttgacc-3′(SEQ ID NO: 18)] and a primer specific to the I-CreI coding sequence for amino acids 14-24 that contains the substitution mutation G19S [G19SF 5′-gccggctttgtggactctgacggtagcatcatc-3′(SEQ ID NO: 25) or G19SR 5′-gatgatgctaccgtcagagtccacaaagccggc-3′(SEQ ID NO: 26)]. The resulting PCR products contain 33 by of homology with each other. The PCR fragments were purified. Finally, approximately 25 ng of each of the two overlapping PCR fragments and 75 ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz and Woods, Methods Enzymol., 2002, 350, 87-96). An intact coding sequence containing the G19S substitution is generated by in vivo homologous recombination in yeast.

The same strategy is used to introduce the I132V substitution into the M1 mutant coding sequence (RTYQS/AYSER according to Table V) using oligonucleotides I132VF: 5′-acctgggtggatcaggttgcagctctgaacgat-3′ (SEQ ID NO: 33) and I132VR: 5′-atcgttcagagctgcaacctgatccacccaggt-3′ (SEQ ID NO: 34).

b) Cloning of Mutants in Vector pCLS1107 in the Yeast Strain Containing the IL2RG3 Target

The yeast strain FYBL2-7B (MAT a, ura3Δ851, trp1Δ63, leu2Δ1, lys2Δ202) containing the IL2RG3 target into yeast reporter vector (pCLS1055, FIG. 5) is transformed with optimized mutants, derived from mutants cleaving the IL2RG3.3 target identified in examples 5 and 6 (Tables VIII and IX), in pCLS1107 vector (FIG. 9), using a high efficiency LiAc transformation protocol. Mutant-target yeasts are used as targets for mating assays as described in example 4.

### 2) Results

The mutations G19S and I132V were incorporated into the M1 mutant (RTYQS/AYSER according to Table V) cleaving the IL2RG3.4 target. Clones resulting from site-directed mutagenesis were mated with 6 yeast strains that (i) contains the IL2RG3 target in a reporter plasmid (ii) expresses a refined mutant derived from mutants cleaving IL2RG3.3. 6 such yeast strains where constructed with mutants .3_R1, .3_R13, .3_R17, .3_R18, .3_R19 and .3_R21 (described in examples 5 and 6, Tables VIII and IX).

Clones were found to trigger cleavage of the IL2RG3 target when mated with such yeast strains (examples are shown in FIG. 13). They were sequenced and the best clones turned out to be four novel endonucleases derived from the M1 mutant cleaving IL2RG3.4 (described in Table X).

Thus, four I-CreI mutants (SEQ ID NO: 101 to 104) derived from the mutant cleaving the IL2RG3.4 target that were able to efficiently cleave the IL2RG3 target when forming heterodimers with optimized mutants derived from mutants cleaving the IL2RG3.3 target, were identified (Table X). Two out of the four optimized mutants contain the G19S or 132V substitution. The two other contain the G19S mutation and other mutations probably resulting from the PCR reaction.

TABLE X
Functional mutant combinations displaying strong cleavage activity for IL2RG3 DNA target

| Optimized mutants derived from | | Optimized mutants derived from | |
|---|---|---|---|
| mutants cleaving IL2RG3.3 | | mutant cleaving IL2RG3.4 | |
| Name | Sequence | Name | Sequence |
| .3_R1 | 26R 31R 33H 44K 68Y 70S | .4_R0 | 30R 32T 44A 68Y 70S 75E 77R 132V |
| | 75E 77V 89A 117G 139R | .4_R1 | 19S 30R 32T 44A 68Y 70S 75E 77R |
| | | .4_R2 | 19S 30R 32T 44A 59A 68Y 70S 75E 77R 111R |
| | | .4_R3 | 19S 30R 32T 44A 60G 68Y 70S 75E 77R |
| .3_R13: | 19S 31R 33H 44K 68Y 70S | .4_R0 | 30R 32T 44A 68Y 70S 75E 77R 132V |
| | 75E 77V 139R | | |
| .3_R17: | 26R 31R 33H 44K 54L 68Y | .4_R0 | 30R 32T 44A 68Y 70S 75E 77R 132V |
| | 70S 75E 77V 139R | .4_R1 | 19S 30R 32T 44A 68Y 70S 75E 77R |
| | | .4_R2 | 19S 30R 32T 44A 59A 68Y 70S 75E 77R 111R |
| | | .4_R3 | 19S 30R 32T 44A 60G 68Y 70S 75E 77R |
| .3_R18: | 31R 33H 44K 68Y 70S 71R | .4_R0 | 30R 32T 44A 68Y 70S 75E 77R 132V |
| | 75E 77V 87L 132V 139R | .4_R1 | 19S 30R 32T 44A 68Y 70S 75E 77R |
| | 147A | .4_R2 | 19S 30R 32T 44A 59A 68Y 70S 75E 77R 111R |
| | | .4_R3 | 19S 30R 32T 44A 60G 68Y 70S 75E 77R |
| .3_R19: | 19S 33H 40Y 44R 68Y 70S | .4_R0 | 30R 32T 44A 68Y 70S 75E 77R 132V |
| | 75D 77T 87L 139R | | |
| .3_R21: | 19S 33H 40Y 44R 68Y 70S | .4_R0 | 30R 32T 44A 68Y 70S 75E 77R 132V |
| | 75D 77T 87L 154G 157G | | |

### EXAMPLE 8

### Refinement of Meganuclease Cleaving the IL2RG3 Target Site by Random Mutagenesis of the I-CreI Mutant Cleaving the IL2RG3.4 Target and Screen in CHO Cells

I-CreI mutants able to cleave the IL2RG3 target in yeast were previously identified by assembly of refined mutant cleaving IL2RG3.4 and refined mutants cleaving IL2RG3.3.

In this example, it was checked if the activity of the meganucleases can be increased and in the same time if the meganucleases are active in CHO cells. The mutants cleaving IL2RG3.4 described in example 7 (Table X) were subjected to random mutagenesis and more efficient variants cleaving IL2RG3 in combination with refined mutants cleaving IL2RG3.3 (identified in example 6) were screened in CHO cells. The screen in CHO cells is an extrachromosomic Single-strand annealing (SSA) based assay where cleavage of the target by the meganucleases induced homologous recombination and expression of a LagoZ reporter gene.

### 1) Materials and Methods

### a) Cloning of IL2RG3 Target in a Vector for CHO Screen

The target was cloned as follow: oligonucleotide corresponding to the target sequence flanked by gateway cloning sequence was ordered from PROLIGO: 5′ tggcatacaagtttcgacctctggtaccagaggtcgacaatcgtctgtca 3′ (SEQ ID NO: 16). Double-stranded target DNA, generated by PCR amplification of the single stranded oligonucleotide, was cloned using the Gateway protocol (INVITROGEN) into CHO reporter vector (pCLS1058, FIG. 14). Cloned target was verified by sequencing (MILLEGEN).

### b) Construction of Libraries by Random Mutagenesis

I-CreI mutants cleaving IL2RG3.4 described in Table X were pooled and randomly mutagenized. Random mutagenesis libraries were constructed by PCR using Mn²⁺ or derivatives of dNTPs as 8-oxo-dGTP and dPTP in two-step PCR process as described in the protocol from JENA BIOSCIENCE GmbH in JBS dNTP-Mutagenesis kit. Primers used are attB1-ICreIFor (5′- ggggacaagtttgtacaaaaaagcaggcttcgaaggagatagaaccatggccaataccaaatataacaaagagttcc-3′; SEQ ID NO: 35) and attB2-ICreIRev (5′-ggggaccactttgtacaagaaagctgggtttagtcggccgccggggaggatttcttcttctcgc-3′; SEQ ID NO: 36). PCR products obtained were cloned in pCDNA6.2 from INVITROGEN (pCLS1069, FIG. 15), a vector for expression in CHO cells, using the Gateway protocol (INVITROGEN).

### c) Re-Cloning of Meganucleases

The ORF of I-SceI, I-CreI N75 and I-CreI mutants cleaving the IL2RG3.3 target identified in example 5 were re-cloned in pCLS1069 (FIG. 15). ORFs were amplified by PCR on yeast DNA using the here above described attB1-ICreIFor and attB2-ICreIRev primers. PCR products were cloned in CHO expression vector pCDNA6.2 from INVITROGEN (pCLS1069, FIG. 15) using the Gateway protocol (INVITROGEN). Resulting clones were verified by sequencing (MILLEGEN).

### d) Extrachromosomal Assay in Mammalian Cells

CHO K1 cells were transfected with Polyfect® transfection reagent according to the supplier's protocol (QIAGEN). 72 hours after transfection, culture medium was removed and 150 μl of lysis/revelation buffer for β-galactosidase liquid assay was added (typically 1 liter of buffer contained: 100 ml of lysis buffer (Tris-HCl 10 mM pH7.5, NaCl 150 mM, Triton X100 0.1%, BSA 0.1 mg/ml, protease inhibitors), 10 ml of Mg 100× buffer (MgCl₂ 100 mM, β-mercaptoethanol 35%), 110 ml ONPG 8 mg/ml and 780 ml of sodium phosphate 0.1M pH7.5). After incubation at 37° C., OD was measured at 420 nm. The entire process is performed on an automated Velocityll BioCel platform. Positives clones resulting of the screen of libraries were secondary screened and verified by sequencing (MILLEGEN).

Per assay, 150 ng of target vector was cotransfected with 12.5 ng of each one of both mutants (12.5 ng of mutant cleaving palindromic IL2RG3.3 target and 12.5 ng of mutant cleaving palindromic IL2RG3.4 target).

### 2) Results

Refined mutants cleaving IL2RG3.4 described in example 7 (Table X) were subjected to another round of optimization. They were pooled, randomly mutagenized on all proteins and a library of new I-CreI variants was cloned in the pCLS1069 vector allowing expression of the mutant in CHO cells (FIG. 15). 1728 clones were screened using the extrachromosomal assay in CHO cells. The screen is done by co-transfection of 3 plasmids in CHO cells: one expressing a variant resulting of random mutagenesis of the mutant cleaving IL2RG3.4, a second expressing a chosen mutant cleaving IL2RG3.3 re-cloned in pCLS 1069 (FIG. 15) and a third one containing the IL2RG3 target cloned in pCLS1058 (FIG. 14). Two I-CreI mutants cleaving IL2RG3.3 were used for the screen of the library: .3_R17 and .3_R14 (26R, 31R, 33H, 44K, 54L, 68Y, 70S, 75E, 77V, 139R and 19S, 33H, 40Y, 44K, 68Y, 70S 75E, 77V, 139R, according to Table IX in example 6).

Eight clones were found to trigger cleavage of the IL2RG3 target in the CHO assay when forming heterodimers with the .3_R17 (26R, 31R, 33H, 44K, 54L, 68Y, 70S, 75E, 77V, 139R) I-CreI mutant in a primary screen. The 8 clones (SEQ ID NO: 105 to 107, 195 and 108 to 111) were validated in a secondary screen (FIG. 16) and sequenced (Table XI). In the secondary screen, the efficiency of those 8 clones was compared to the initial M1 mutant co-expressed with .3_R17 and 5 out of 8 displayed a stronger activity against IL2RG3 (in bold in Table XI).

In conclusion, five new refined mutants were identified that were able to cleave the IL2RG3 target when forming heterodimers with the 26R, 31R, 33H, 44K, 54L, 68Y, 70S, 75E, 77V, 139R I-CreI mutant with an efficacy in the CHO assay superior to the one observed with the heterodimer formed by the initial M1 mutant (RTYQS/AYSER according to Table V) and the 26R, 31R, 33H, 44K, 54L, 68Y, 70S, 75E, 77V, 139R I-CreI mutant.

TABLE XI
I-CreI mutants displaying cleavage activity for IL2RG3 DNA
target when forming heterodimers with .3_R17 (26R, 31R,
33H, 44K, 54L, 68Y, 70S, 75E, 77V, 139R I-CreI mutant).

| Name | Sequence |
|---|---|
| .4_R4 | 19S 30R 32T 44A 59A 68Y 70S 75E 77R 111R 122Y |
| .4_R5 | 19S 30R 32T 44A 59A 68Y 70S 75E 77R 103Y 107R |
| .4_R6 | 19S 30R 32T 44A 60G 68Y 70S 75E 77R 96R 98R |
| .4_R7 | 19S 30R 32T 44A 68Y 70S 75E 77R |
| .4_R8 | 19S 30R 32T 44A 60G 68Y 70S 75E 77R 135Q 153G 164G |
| | 165P |
| .4_R9 | 19S 30R 32T 44A 60G 68Y 70S 75E 77R 156R |
| .4_R10 | 19S 30R 32T 44A 52C 68Y 70S 75E 77R |
| .4_R11 | 19S 30R 32T 44A 59A 68Y 70S 75E 77R 82R 111R 140A |

### EXAMPLE 9

### Validation of IL2RG3 Target Cleavage in an Extrachromosomic Model in CHO Cells

Several I-CreI refined mutants able to efficiently cleave the IL2RG3 target in yeast or CHO when forming heterodimers were identified in examples 5, 6 7 and 8. In order to characterize the heterodimer displaying the maximal efficacy to cleave the IL2RG3 target in CHO cells, the efficiency of all combinations of mutants to cut the IL2RG3 target was compared, using the extrachromosomal assay in CHO cells.

### 1) Materials and Methods

The experimental procedures are described in example 8.

### 2) Results

Mutants described in examples 5, 6 and 7 were first re-cloned in pCLS1069. Then, in order to characterize the heterodimer displaying the maximal efficacy to cleave the IL2RG3 target in CHO cells, refined I-CreI mutants cleaving the IL2RG3.3 or IL2RG3.4 targets (described in examples 5, 6, 7 and 8) were tested together in heterodimer against the IL2RG3 target in the CHO extrachromosomal assay.

The maximal values where observed with heterodimers formed by .3_R27 or .3_R28 (31R, 33H, 44K, 68Y, 70S, 75E, 77V, 80G, 132V, 139R or 31R, 33H, 44K, 68Y, 70S, 75E, 77V, 132V, 139R, as described in Table IX) combined with .4_R2, .4_R5, .4_R9 or .4_R11 I-CreI mutants (described in Tables X and XI). The FIG. 17 shows the results obtained for those 8 heterodimers against the IL2RG3 target in CHO cells assay, compared to the activity of I-SceI against its target. In conclusion, 6 combinations of I-CreI mutants (Table XII) were identified that were able to cut the IL2RG3 target in CHO cells with an activity similar to that of I-SceI against the I-SceI target (tagggataacagggtaat: SEQ ID NO: 37).

TABLE XII
I-CreI mutants combinations displaying the maximal efficiency
of cleavage of the IL2RG3 target in CHO cells.

| Refined mutant | Optimized mutant derived from |
|---|---|
| cleaving IL2RG3.3 | mutants cleaving the IL2RG3.4 target |
| .3_R27 | .4_R9: 19S 30R 32T 44A 60G 68Y 70S 75E |
| 31R 33H 44K | 77R 156R |
| 68Y 70S 75E | .4_R11: 19S 30R 32T 44A 59A 68Y 70S 75E |
| 77V 80G 132V | 77R 82R 111R 140A |
| 139R | |
| .3_R28 | .4_R2: 19S 30R 32T 44A 59A 68Y 70S 75E |
| 31R 33H 44K | 77R 111R |
| 68Y 70S 75E | .4_R5: 19S 30R 32T 44A 59A 68Y 70S 75E |
| 77V 132V 139R | 77R 103Y 107R |
| | .4_R9: 19S 30R 32T 44A 60G 68Y 70S 75E |
| | 77R 156R |
| | .4_R11: 19S 30R 32T 44A 59A 68Y 70S 75E |
| | 77R 82R 111R 140A |

### EXAMPLE 10

### Refinement of Meganucleases Cleaving the IL2RG3 Target Site by Random Mutagenesis of Protein Cleaving IL2RG3.4 and Assembly with Refined Proteins Cleaving IL2RG3.3

I-CreI mutants able to cleave the IL2RG3 target were previously identified by assembly of refined mutants cleaving IL2RG3.4 and refined mutants cleaving IL2RG3.3 (examples 5 to 9). In this example, the M1 mutant cleaving IL2RG3.4 (example 3, Table V) was randomly mutagenized on the whole protein and screened in yeast for more efficient variants cleaving IL2RG3 in combination with refined mutants cleaving IL2RG3.3 described in example 6.

### 1) Material and Methods

### a) Random Mutagenesis

The experimental procedure is as described in example 5. In this example, random mutagenesis was performed on the whole protein using Mn²⁺ on the M1 mutant. 75 ng of PCR fragment and 75 ng of pCLS0542 linearized by digestion with NcoI/EagI were used to generate the library of variants by in vivo homologous recombination in yeast.

b) Cloning of Mutants in Vector pCLS1107 in the Yeast Strain Containing the IL2RG3 Target

The experimental procedure is as described in example 5. In this example, the yeast strain FYBL2-7B containing the IL2RG3 target is transformed with mutants cutting IL2RG3.3 in pCLS1107 vector.

### c) Re-Cloning of Meganucleases

The experimental procedure is as described in example 8.

### d) Validation of IL2RG3 Target Cleavage in an Extrachromosomic Model in CHO K1 Cells

The experimental procedure is as described in example 8.

### 2) Results

New I-CreI mutants able to efficiently cleave IL2RG3 target when forming heterodimers with mutants cleaving the IL2RG3.3 target, were identified. The M1 mutant cleaving IL2RG3.4 (RTYQS/AYSER according to Table V) was randomly mutagenized by PCR on all protein and transformed into yeast. 2232 transformed yeast clones were then mated with yeast strains that (i) contain the IL2RG3 target in a reporter plasmid (ii) express the .3_R17 (I-CreI 26R 31R 33H 44K 54L 68Y 70S 75E 77V 139R according to Table IX) or the .3_R19 mutant (I-CreI 19S 33H 40Y 44R 68Y 70S 75D 77T 87L 139R according to Table IX), variants cleaving the IL2RG3.3 target as described in example 6. 22 clones were found to trigger cleavage of the IL2RG3 target when mated with such yeast strain. After sequencing, they turned out to be 12 novel endonucleases (SEQ ID NO: 128 to 139) derived from the M1 mutant cleaving IL2RG3 in combination with .3_R17 and .3_R19 (Table XIII).

TABLE XIII
Functional mutant combinations displaying strong
cleavage activity for IL2RG3 DNA target.

| Refined mutant | Optimized* mutant derived from |
|---|---|
| cleaving IL2RG3.3 | M1 mutant cleaving IL2RG3.4 |
| .3_R17 | M1_24V: 24V 30R 32T 44A 68Y 70S 75E 77R |
| 26R 31R 33H | M1_24T: 24T 30R 32T 44A 68Y 70S 75E 77R |
| 44K 54L 68Y | M1_34R: 30R 32T 34R 44A 68Y 70S 75E 77R |
| 70S 75E 77V | M1_43I: 30R 32T 43I 44A 68Y 70S 75E 77R |
| 139R | M1_64A: 30R 32T 44A 64A 68Y 70S 75E 77R |
| | M1_100R: 30R 32T 44A 68Y 70S 75E 77R |
| | 100R |
| | M1_132V: 30R 32T 44A 68Y 70S 75E 77R |
| | 132V |
| | M1_43L: 30R 32T 43L 44A 68Y 70S 75E 77R |
| | M1_31R_34R: 30R 31R 32T 34R 44A 68Y 70S |
| | 75E 77R |
| | M1_57R_107E: 30R 32T 44A 57R 68Y 70S 75E |
| | 77R 107E |
| | M1_103D: 30R 32T 44A 68Y 70S 75E 77R 103D |
| | M1_117K: 30R 32T 44A 68Y 70S 75E 77R 117K |
| .3_R19 | M1_24V: 24V 30R 32T 44A 68Y 70S 75E 77R |
| 19S 33H 40Y | M1_24T: 24T 30R 32T 44A 68Y 70S 75E 77R |
| 44R 68Y 70S | M1_34R: 30R 32T 34R 44A 68Y 70S 75E 77R |
| 75D 77T 87L | M1_43I: 30R 32T 43I 44A 68Y 70S 75E 77R |
| 139R | M1_64A: 30R 32T 44A 64A 68Y 70S 75E 77R |
| | M1_100R: 30R 32T 44A 68Y 70S 75E 77R |
| | 100R |
| | M1_132V: 30R 32T 44A 68Y 70S 75E 77R |
| | 132V |
| | M1_43L: 30R 32T 43L 44A 68Y 70S 75E 77R |
| | M1_31R_34R: 30R 31R 32T 34R 44A 68Y 70S |
| | 75E 77R |
| | M1_57R_107E: 30R 32T 44A 57R 68Y 70S 75E |
| | 77R 107E |
| | M1_103D: 30R 32T 44A 68Y 70S 75E 77R 103D |
| | M1_117K: 30R 32T 44A 68Y 70S 75E 77R 117K |

*Mutations resulting from random mutagenesis are in bold.

We focused on M1_{—}24V showing very efficient cleavage activity in yeast on IL2RG3 target. In FIG. 20, cleavage efficiency of IL2RG3 target in yeast was compared for several combinations of mutants: .4_R5, .4_R9 (Table XI) and M1_{—}24V (Table XIII) in combination with .3_R17, .3_R25 and .3_R28 I-CreI variants described in example 6 (Table IX). The best cleavage activity was observed with the combination M1_{—}24V and .3_R17_I-CreI mutants (FIG. 20).

Meganucleases were re-cloned in pCLS1069 for .3_R28, .4_R5 and .4_R9 and in pCLS1768 for .3_R17, .3_R25 and M1_{—}24V (pCLS1768 corresponds to pCLS1069 without T7 origin, as described in FIG. 21). During the re-cloning step, mutations appeared on .3_R25 I-CreI variant leading to 3 novel endonucleases (.3_R25a, .3_R25b and .3_R25c described in Table XIV).

TABLE XIV
Sequence of meganucleases derived from .3_R25 I-CreI variant.

| name | Sequence (SEQ ID NO: 140 to 142) |
|---|---|
| .3_R25a | 19S 33H 40Y 44R 68Y 70S 71R 75E 77V 132V 139R |
| .3_R25b | 19S 33H 40Y 44R 68Y 70S 75D 77T 127N 132V |
| .3_R25c | 19S 33H 40Y 44R 68Y 70S 71R 75E 77V 132V |

* Mutations resulting from re-cloning step are in bold.

The efficiency of all the combinations of these re-cloned mutants to cleave the IL2RG3 target was compared in CHO K1 cells with the activity of I-CreI N75 and I-SceI on their respective targets (named C1234 and S1234) using an extrachromosomal SSA assay as described in example 8.

We identify new combinations of I-CreI mutants cleaving the IL2RG3 target with an activity similar to that of I-SceI against the I-SceI target and I-CreI N75 against the I-CreI target (FIG. 22). Efficient combinations of I-CreI variants against the IL2RG3 target are: .3_R28 co-expressed with .4_R5, .4_R9 or M1_{—}24V; .3_R17 co-expressed with .4_R5, .4_R9 or M1_{—}24V and .3_R25a or .3_R25c co-expressed with M1_{—}24V. .3_R25b in combination with M1_{—}24V is less active. Combinations of .3_R25a, .3_R25b or .3_R25c co-expressed with .4_R5 or .4_R9 are inactive. In this extra-chromosomal SSA assay in CHO K1 cells, the best efficiency of IL2RG3 target cleavage was observed with the combination .3_R25a and M1_{—}24V.

### EXAMPLE 11

### Refinement of Meganucleases Cleaving the IL2RG3 Target Site by Site-Directed Mutagenesis of Refined Protein Cleaving IL2RG3.4 and Assembly with Refined Proteins Cleaving IL2RG3.3

The M1_{—}24V I-CreI 24V 30R 32T 44A 68Y 70S 75E 77R mutant (Table XIII) described in example 10 was subjected to a next step of optimization by introducing selected amino-acid substitutions and screening for more efficient variants cleaving IL2RG3 in combination with .3_R17 and .3_R25 refined mutants cleaving IL2RG3.3 identified in example 6.

Five amino-acid substitutions have been found in previous studies to enhance the activity of I-CreI derivatives (G19S, F54L, F87L, V105A and I132V-see example 6). We also introduced the E80K substitution.

### 1) Material and Methods

### Site Directed Mutagenesis

Site directed mutagenesis on M1_{—}24V I-CreI mutant was performed by PCR using the experimental procedure described in example 6. For the E80K substitution we used the following pair of oligonucleotides:

*E80KF:
(SEQ ID NO: 151)
5′-ttaagcaaaatcaagccgctgcacaacttcctg-3′
and
E80KR:
(SEQ ID NO: 152)
5′-caggaagttgtgcagcggcttgattttgcttaa-3′

### 2) Results

Yeast strains containing the M1_{—}24V I-CreI variant with one or two of the six amino-acid substitutions were screened for IL2RG3 target cleavage efficiency by mating with a yeast strain that (i) contains the IL2RG3 target in a reporter plasmid (ii) expresses the .3_R17, .3_R25 or .3_R28 I-CreI mutant (according to Table IX).

New I-CreI mutants (described in Table XV) able to efficiently cleave the IL2RG3 target when forming heterodimers with .3_R17 and .3_R25 I-CreI mutants were identified (screen results examples are shown in FIG. 23).

TABLE XV
Functional mutant combinations displaying strong
cleavage activity for IL2RG3 DNA target.

| | Optimized* mutant derived from |
|---|---|
| Refined mutant | M1 mutant cleaving IL2RG3.4 |
| cleaving IL2RG3.3 | (SEQ ID NO: 143 to 148) |
| .3_R17 | 24V 30R 32T 44A 68Y 70S 75E 77R 132V |
| 26R 31R 33H | 24V 30R 32T 44A 68Y 70S 75E 77R 80K |
| 44K 54L 68Y | 24V 30R 32T 44A 54L 68Y 70S 75E 77R |
| 70S 75E 77V | 24V 30R 32T 44A 68Y 70S 75E 77R 87L |
| 139R | 24V 30R 32T 44A 68Y 70S 75E 77R 105A |
| | 24V 30R 32T 44A 68Y 70S 75E 77R 105A 132V |
| .3_R25 | 24V 30R 32T 44A 68Y 70S 75E 77R 132V |
| 19S 33H 40Y | 24V 30R 32T 44A 68Y 70S 75E 77R 80K |
| 44R 68Y 70S | 24V 30R 32T 44A 54L 68Y 70S 75E 77R |
| 75E 77V 132V | 24V 30R 32T 44A 68Y 70S 75E 77R 87L |
| | 24V 30R 32T 44A 68Y 70S 75E 77R 105A |
| | 24V 30R 32T 44A 68Y 70S 75E 77R 105A 132V |

*Mutations resulting from site-directed mutagenesis are in bold.

### EXAMPLE 12

### KI Matrix Construction for the Genome Engineering at the IL2RG Gene in Human Cell Lines

I-CreI refined mutants able to efficiently cleave in yeast and in mammalian cells (CHO K1 cells) the IL2RG3 target located in intron 4 of the human IL2RG gene have been identified in previous examples. Lot of mutations have been described in the human IL2RG gene causing X-SCID syndrome. Among them, about half are located downstream of the IL2RG3 target (FIG. 19).

The combination of meganucleases cleaving the IL2RG3 target can be used to correct mutations in the IL2RG gene in patient cells by cleavage followed by homologous recombination using a repair matrix. To test the efficiency of the IL2RG meganucleases to correct hIL2RG, an exon Knock-in matrix (KI matrix) was designed.

### Materials and Methods

### Knock-In (KI) Matrix

The Knock-in matrix is an exon knock-in strategy using a cDNA containing exons 5 to 8 of hIL2RG (cDNA fragment of 520 bp from 609 to 1128 in mRNA human IL2RG sequence NM_{—}000206) cloned between two human IL2RG homology arms (LH of 1268 bp from 130 to 1398 and RH of 1717 bp from 1740 to 3451 in the genomic sequence NC_{—}000023.9) (FIG. 24). The resulting plasmid is pCLS2037 (FIG. 25). The homology arms are amplified from genomic DNA purified from human cell lines (HEK-293 for LH and EBV transformed human B cells line for RH). The coding sequence of the neomycin resistance gene (Neo) is operatively linked to an IRES region and to the SV40 polyA signal. The neomycin expression cassette (IRES_Neo_pA) can be released and replaced by a pA site by enzymatic digestion. The thymidine kinase from HSV under the control of the EF1α promoter cloned after the RH arm can be used to eliminate clones with random integration of the KI matrix.

A second gene targeting vector was constructed with the same strategy of exons knock-in (pCLS1976, FIG. 24). In pCLS1976, 3% of heterology in nucleotides was introduced in the cDNA exons 5 to 8.

### EXAMPLE 13

### Making of Meganucleases Cleaving the IL2RG3.6 Target Sequence by Using a Sequential Combinatorial Approach

The IL2RG3.6 DNA sequence differs only from IL2RG3.4 by the four central base pairs that are called 2NN_{—}2NN. IL2RG3.4 carries GTAC as the C1221 target while IL2RG3.6 has a TCTC sequence like the IL2RG3 target (FIG. 4) and is therefore more difficult to cleave by an I-CreI derived mutant. We have previously observed that the association of a mutant cleaving a palindromic target with a wild-type 2NN_{—}2NN sequence with a mutant cleaving the other palindromic target will increase the probability of cleavage of the target of interest.

To obtain such an IL2RG3.6 cutter, a strategy based on a sequential combinatorial approach was used. This approach is different from the traditional combinatorial approach developed in example 3 to obtain meganucleases cleaving the IL2RG3.4 target. In example 3, mutations of mutants cleaving the 10GAA_P target and mutants cleaving the 5AGG_P target were combined to obtain mutants able to cleave IL2RG3.4. In the sequential combinatorial approach, we looked first for mutants cleaving the IL2C_P target (FIG. 4). This palindromic target is identical to the 5AGG_P target but with the bases at positions ±11 and ±7 of the IL2RG3.4 target (FIG. 4). IL2C_P cutters were then chosen to create different mutant libraries degenerated at I-CreI amino acid positions 28, 30, 32 and 33 that were screened using our yeast screening assay against the IL2RG3.4 target. Instead of combining two mutations sets like in example 3, the concept of the sequential approach is to fix one mutation set (here mutations allowing for IL2C_P cleavage) before looking for the second mutation set. Finally, a site-directed mutagenesis was then performed on IL2RG3.4 proteins obtained by the sequential method to obtain cleavage activity toward the IL2RG3.6 target.

### 1) Material and Methods

### a) Construction of the Sequential Mutant Libraries SeqLib1 and SeqLib2

The two mutant libraries SeqLib1 and SeqLib2 were generated from the DNA of a pool of three IL2C_P cutters. To build SeqLib1, which contains mutations at positions 30 and 33, two separate overlapping PCR reactions were carried out that amplify the 5′ end (aa positions 1-41) or the 3′ end (aa positions 34-166) of the I-CreI derived mutants coding sequence. For the 3′ end, PCR amplification is carried out using a primer specific to the pCLS0542 vector (Gal10R 5′-acaaccttgattggagacttgacc-3′; SEQ ID NO: 18) and a primer specific to the I-CreI coding sequence for amino acids 34-43 (10RG34For 5′-aagtttaaacatcagctaagcttgaccttt-3′; SEQ ID NO: 153). For the 5′ end, PCR amplification is carried out using a primer specific to the pCLS0542 vector (Gal10F 5′-gcaactttagtgctgacacatacagg-3′: SEQ ID NO: 17) and a primer specific to the I-CreI coding sequence for amino acids 25-41 (10RG34Rev1 5′-caagcttagctgatgtttaaacttmnnagactgmnntggtttaatctgagc-3′; SEQ ID NO: 154). The MNN code in the oligonucleotide resulting in a NNK codon at positions 30 and 33 allows the degeneracy at these positions among the 20 possible amino acids. The SeqLib2 library that contains mutations at positions 28, 32 and 33 was built using the same method but with the use of the primer 10RG34Rev2 (5′-caagcttagctgatgtttaaacttmbnmbnctggtttggmbnaatctgagc-3′; SEQ ID NO: 155) instead of 10RG34Rev1. The MBN code in the oligonucleotide resulting in a NVK codon at positions 28, 32 and 33 allows the degeneracy at these positions among all the 20 possible amino acids but F, L, M, I and V. Then, for both libraries, 25 ng of each of the two overlapping PCR fragments and 75ng of vector DNA (pCLS0542) linearized by digestion with NcoI and EagI were used to transform the yeast Saccharomyces cerevisiae strain FYC2-6A (MATα, trp1Δ63, leu2Δ1, his3Δ200) using a high efficiency LiAc transformation protocol (Gietz R D and Woods R A Transformation of yeast by lithium acetate/single-stranded carrier DNA/polyethylene glycol method. Methods Enzymol. 2002; 350:87-96). An intact coding sequence containing mutations at desired positions is generated by in vivo homologous recombination in yeast.

### b) Site-Directed Mutagenesis

The I132V and E80K mutations were introduced on a DNA pool constituted by the Seq4, Seq5 and Seq7 I-CreI mutants as described in examples 6 and 11.

### 2) Results

The yeast screening of 36 I-CreI mutants able to cleave the 5AGG_P target against the IL2C_P target gave some positive clones (FIG. 26). Three positive mutants were isolated. They all have the I24V mutation and have respectively the following sequences: TRSER, TYSER, RYSET, where letters indicate amino acids at positions 44, 68, 70, 75 and 77 (for example, TRSER stands for T44, R68, S70, E75 and R77). Using the DNA of these three positive clones toward the IL2C_P target, two different mutant libraries were then built by degenerating amino acids positions 30 and 33 for the first library (SeqLib1) and 28, 32 and 33 for the second library (SeqLib2). Yeast screening of 1116 clones from library 1 against the IL2RG3.4 target yielded 6 positives clones with a unique sequence (FIG. 27) and the screening of 2232 clones from library 2 gave one positive clone. The sequence of the seven IL2RG3.4 cutters is given in Table XVI.

TABLE XVI
Sequences of the seven IL2RG3.4 cutters
obtained by a sequential combinatorial method.
Letters indicate amino acids at positions
28, 30, 32, 33, 38, 40, 44, 68, 70, 75 and 77

| Clones | Sequence (SEQ ID NO: 156 to 162) |
|---|---|
| Seq1 | V24 - KRSYQS/TRSER |
| Seq2 | V24 - KRSNQS/TYSER |
| Seq3 | V24 - KRSAQS/TRSER |
| Seq4 | V24 - KRSVQS/TRSER + Q50R |
| Seq5 | V24 - KRSSQS/RYSET + V129A |
| Seq6 | V24 - KRSVQS/TYSER |
| Seq7 | V24 - KNGHQS/TRSER |

As the cleavage activity toward the IL2RG3.4 target for the seven clones Seq1 to Seq7 is still relatively weak, the mutations E80K and I132V were introduced by site-directed mutagenesis on a pool of mutants constituted by the Seq4, Seq5 and Seq7 clones. The screening of the resulting mutants gave very strong cutters against the IL2RG3.4 target and three clones among them with a unique sequence given in Table XVII were able to cleave the IL2RG3.6 target (FIG. 28).

TABLE XVII
Sequence of the three IL2RG3.6 cutters.
The clones are ranked with
a decreasing cleavage activity

| IL2RG3.6 | |
|---|---|
| Cutters | Sequence (SEQ ID NO: 163 to 165) |
| Amel1 | V24 - KNGHQS/TRSER + Q50R, I132V |
| Amel2 | V24 - KNGHQS/TRSER + E80K |
| Amel3 | V24 - KRSVQS/TRSER + Q50R, E80K, V129A |

Through a refinement process led by site-directed mutagenesis, three I-CreI derived mutants able to cleave the IL2RG3.6 have been obtained. The initial IL2RG3.4 cutters have been isolated by using a sequential combinatorial approach, which validates this concept described in the introduction of this example. The three IL2RG3.6 cutters can now be used in co-expression with IL2RG3.3 mutants to cleave the IL2RG3 target.

1-40. (canceled)

41. A method of cleaving a DNA target sequence from a human IL2RG gene comprising contacting said DNA target sequence with an I-CreI variant to thereby cleave said DNA target sequence
wherein said I-CreI variant comprises a first monomer and a second monomer which are associated to form an active form,
wherein said I-CreI variant comprises at least two substitutions in at least one of the monomers,
wherein at least one substitution is of a residue in the range of positions 26 to 40 of I-CreI and at least one substitution is of a residue in the range of positions 44 to 77 of I-CreI and
wherein said DNA target sequence is at least one sequence selected from the group consisting of SEQ ID NO: 5 to 9 and 116 to 119.

42. The method of claim 41, wherein said at least one substitution of a residue in the range of 26 to 40 of I-CreI is at least one substitution of a residue selected from the group consisting of positions 26, 28, 30, 32, 33, 38 and 40.

43. The method of claim 41, wherein said at least one substitution of a residue in the range of 44 to 77 of I-CreI is at least one substitution of a residue selected from the group consisting of positions 44, 68, 70, 75 and 77.

44. The method of claim 41, wherein said substitutions comprise replacing the wild-type amino acids with an amino acid selected from the group consisting of A, D, E, G, H, K, N, P, Q, R, S, T, Y, C, W, L and V.

45. The method of claim 41, wherein said I-CreI variant further comprises at least one substitution of a residue selected from the group consisting of positions 2, 4, 7, 8, 19, 24, 31, 34, 39, 43, 50, 52, 54, 57, 59, 60, 64, 71, 79, 80, 82, 87, 89, 96, 98, 100, 103, 105, 107, 111, 117, 121, 122, 127, 129, 132, 135, 139, 140, 143, 147, 153, 154, 156, 157, 159, 160, 162, and 163 of I-CreI.

46. The method of claim 41, wherein said I-CreI variant further comprises at least one substitution selected from the group consisting of: N2D, K4E, K7E, E8G, G19S, G19A, I24V, I24T, Q26R, Q31R, K34R, L39I, F43L, F43I, Q50R, R52C, F54L, K57R, V59A, D60G, V64A, G71R, S79G, E80K, E80G, K82R, F87L, T89A, K96R, K98R, K100R, N103Y, N103D, V105A, K107R, K107E, Q111R, E117G, E117K, K121R, F122Y, T127N, V129A, I132V, I132T, L135Q, K139R, T140A, T143I, T147A, D153G, S154G, S156R, E157G, K159E, K159R, K160G, S162F, S162P and P163L.

47. The method of claim 41, wherein said I-CreI variant further comprises at least one substitution selected from the group consisting of: G19S, I24V, F54L, E80K, F87L, V105A and I132V.

48. The method of claim 41, which comprises substitution of the aspartic acid in position 75 of I-CreI.

49. The method of claim 48, wherein position 75 of I-CreI is substituted with an asparagine residue.

50. The method of claim 41, wherein said variant is a homodimer.

51. The method of claim 41, wherein said variant is a heterodimer, resulting from the association of a first and a second monomer having different mutations in positions 26 to 40 and 44 to 77 of I-CreI.

52. The method of claim 51, wherein the first and the second monomer of said I-CreI variant, respectively, have amino acids at positions 28, 30, 32, 33, 38, 40 and 44, 68, 70, 75, 77, which are selected from the group consisting of: KNSTQQ/NYSYQ and SNSYRK/DNSNI, KHTCRS/QRDNR and KNDYYS/QRSHY, KRANQE/YRSQI and KNSCAS/NRSYN, KNSTQQ/RYSEY and KNTYQS/DYSSR, KNSSRE/LRNNI and KDSRTS/AYSYK, KNSRNQ/YRSDV and KNSTAS/QYSRQ, KSSCQA/AYSYI and KNTYWS/AYSYK, KNRDQS/DNSNI and KNSTAS/AYSYK.

53. The method of claim 51, wherein the first monomer has amino acids at positions 28, 30, 32, 33, 38, 40 and 44, 68, 65, 77, which are selected from the group consisting of: KNSRQY/RYSDT, KNSHQS/KYSEV, KNSRQS/RYSDT, KNSHQY/RYSDT, KNSHQY/KYSEV, KNSRQY/RYSEV, KNSHQY/RYSEV and the second monomer has amino acids at positions 28, 30, 32, 33, 38, 40 and 44, 68, 65, 77, which are selected from the group consisting of: KRTYQS/AYSER, KRSYQS/TRSER, KRSNQS/TYSER, KRSAQS/TRSER, KRSVQS/TRSER, KRSSQS/RYSET and KNGHQS/TRSER.

54. The method of claim 51, wherein the first and the second monomer, respectively, have amino acids at positions 28, 30, 32, 33, 38, 40 and 44, 68, 65, 77, and at additional positions, which are selected from the group consisting of:
KNSHQS/KYSEV+26R+31R+54L+139R (first monomer) and KRTYQS/AYSER+19S+59A+103Y+107R, KRTYQS/AYSER+19S+60G+156R or KRTYQS/AYSER+24V (second monomer);
KNSHQS/KYSEV+31R+80G+132V+139R (first monomer) and KRTYQS/AYSER+19S+60G+156R or KRTYQS/AYSER+19S+59A+82R+111R+140A (second monomer),
KNSHQS/KYSEV+31R+132V+139R (first monomer) and KRTYQS/AYSER+19S+59A+111R, KRTYQS/AYSER+19S+59A+103Y+107R, KRTYQS/AYSER+19S+60G+156R, KRTYQS/AYSER+19S+59A+82R+111R+140A or KRTYQS/AYSER+24V (second monomer),
KNSHQY/RYSEV+19S+132V, KNSHQY/RYSEV+19S+71R+132V+139R or KNSHQY/RYSEV+19S+71R+132V (first monomer) and KRTYQS/AYSER+24V (second monomer), and
KNSHQS/KYSEV+26R+31R+54L+139R or KNSHQY/RYSEV+19S+132V (first monomer) and KRTYQS/AYSER+24V+132V, KRTYQS/AYSER+24V+80K, KRTYQS/AYSER+24V+54L, KRTYQS/AYSER+24V+87L, KRTYQS/AYSER+24V+105A or KRTYQS/AYSER+24V+105A+132V (second monomer).

55. The method of claim 51, wherein the first monomer and the second monomer, respectively, are selected from the following pairs of sequences: SEQ ID NO: 38 and 43; SEQ ID NO: 39 and 44; SEQ ID NO: 40 and SEQ ID NO: 45; SEQ ID NO: 41 and SEQ ID NO: 46; SEQ ID NO:42 and SEQ ID NO: 47; SEQ ID NO: 120 and 121, SEQ ID NO: 122 and 123, SEQ ID NO: 124 and 125, SEQ ID NO: 126 and 127, and SEQ ID NO: 67 to 100, 140 to 142 (first monomer) and any of the SEQ ID NO: 101 to 111, 128 to 139, 143 to 148 and 156 to 165 (second monomer).

56. The method of claim 51, wherein at least one of the two I-CreI monomers has at least 95% sequence identity with one of the sequences selected from the group consisting of: SEQ ID NO: 38-47, 67-111, 120-148, and 156-165.

57. The method of claim 51, wherein the first and the second monomer, respectively, further comprises the D137R mutation and the R51D mutation.

58. The method of claim 51, wherein the first monomer further comprises the E8R or E8K and E61R mutations and the second monomer further comprises the K7E and K96E mutations.

59. The method of claim 41 wherein said variant is a single-chain chimeric meganuclease comprising two I-CreI monomers.

60. The method of claim 59 wherein said chimeric meganuclease comprises a first monomer and a second monomer wherein each monomer has the same substitutions.

61. The method of claim 59 wherein said chimeric meganuclease comprises a first monomer and a second monomer wherein each monomer has at least one different substitution in positions 26 to 40 and 44 to 77 of I-CreI.

62. The method of claim 41, wherein said I-CreI variant is made from the starting scaffold of SEQ ID NO: 1.

63. The method of claim 41, wherein said I-CreI variant is made from the starting scaffold of SEQ ID NO: 4.

64. The method of claim 41 wherein said contacting is in a cell.

65. The method of claim 41 wherein said I-CreI variant is expressed in a cell from a polynucleotide encoding said I-CreI variant.
